(19) United States
(12) Patent Application Publication
(10) Pub. No.: US 2009/0036422 A1
(43) Pub. Date: Feb. 5, 2009

(54) MONOAMIDE DERIVATIVES AS OREXIN RECEPTOR ANTAGONISTS

(76) Inventors: Henner Knust, Rheinfelden (DE); Matthias Nettekoven, Grenzach-Wyhlen (DE); Emmanuel Pinard, Linsdorf (FR); Olivier Roche, Folgensbourg (FR); Mark Rogers-Evans, Bottmingen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/178,688

(22) Filed: Jul. 24, 2008

(30) Foreign Application Priority Data
Aug. 2, 2007 (EP) 07113702.0

(51) Int. Cl. A61K 31/397; A61K 31/165; A61K 31/52; A61K 31/437; C07C 233/88; C07D 471/04; C07D 487/04; C07D 491/107

(52) U.S. Cl. 514/210.16; 514/617; 514/263.1; 514/303; 564/123; 546/118; 544/280; 548/950

(57) ABSTRACT

The present invention relates to compounds of formula

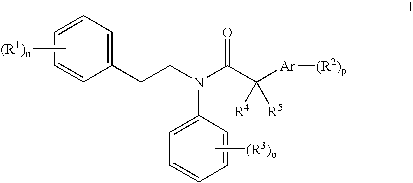

wherein
Ar, R¹, R², R³, R⁴, R⁵, n, o, and p are as defined herein
or to pharmaceutically suitable acid addition salts, optically pure enantiomers, racemates or diastereomeric mixtures thereof. The compounds of formula I can be used for the treatment of sleep disorders, such as sleep apnea, narcolepsy, insomnia, parasomnia, jet lag syndrome, circadian rhythms disorder or sleep disorders associated with neurological diseases.

### PRIORITY TO RELATED APPLICATION(S)

This application claims the benefit of European Patent Application No. 07113702.0, filed Aug. 2, 2007, which is hereby incorporated by reference in its entirety.

### BACKGROUND OF THE INVENTION

Orexins (hypocretins), a family of hypothalamic neuropeptides, play an important role in modulating feeding behavior, energy homeostasis and the sleep-wake cycle (Siegel, Annu. Rev. Psychol., 55, 125-148, 2004). The orexin-A/hypocretin1 (OX-A, 33 amino acids) and orexin-B/hypocretin2 (OX-B, 28 amino acids) are derived from the same precursor by proteolytic processing of 130 amino acids prepro-orexin (de Lecea et al., Proc Natl Acad Sci USA, 95, 322-327, 1998; Sakurai T. et al., Cell, 92, 573-585, 1998). The orexin levels show a diurnal variation being highest during the active cycle. Two receptor subtypes termed orexin-1 receptor (OX₁R) and orexin-2 receptor (OX₂R) have been identified. The characterization of both receptors in binding and functional assays demonstrated that OX₂R is a non-selective receptor for both OX-A and -B, whereas OX₁R is selective for OX-A, conversely OX-A is a non-selective neuropeptide and binds with similar affinities to OX₁R and OX₂R, while OX-B is selective and has a higher affinity for OX₂R (Sakurai T. et al., Cell, 92, 573-585, 1998). Both receptors belong to the class A family of G-protein-coupled receptors (GPCRs) that couple via G_{q/11} to the activation of phospholipase C leading to phosphoinositide (PI) hydrolysis and elevation of intracellular Ca²⁺ levels. However, it has been shown that OX2R could also couple via G_{i/o} to cAMP pathway (Sakurai, Regulatory Peptides, 126, 3-10, 2005). Northern blot analysis of adult rat tissues showed that the prepro-orexin mRNA is detected exclusively in the brain (except for a small amount in the testis) and that the OX₁R and OX₂R transcripts are also exclusively detected in the brain (Sakurai T. et al., Cell, 92, 573-585, 1998). Similar results were obtained using human multiple tissue Northern blot. Distribution studies in rat brain using in situ hybridization and immunohistochemistry have shown that orexin neurons are found only in the lateral hypothalamic area with their projections to the entire CNS (Peyron et al., J Neurosci, 18, 9996-10015, 1998; Nambu et al., Brain Res., 827, 243-60, 1999). In addition, both OX₁ and OX₂ receptors are present in brain regions important for the regulation of sleep/wakefulness.

A disrupted orexin system is suggested to be the cause of narcolepsy based on following lines of evidence: (a) Prepro-orexin knockout mice possessed a phenotype with characteristics remarkably similar to narcolepsy (Chemelli et al., Cell, 98, 437-451, 1999), (b) a mutation (canarc-1), which disrupts the gene encoding OX₂R, was found to be responsible for canine narcolepsy (Lin et al., Cell, 98, 365-376, 1999), (c) lack of OX-A and OX-B was observed in human narcoleptic patients (Nishino et al., Lancet, 355, 39-40, 2000; Peyron et al., Nature Medicine, 6, 991-997, 2000), (d) it has been shown that Modafinil, an anti-narcoleptic drug with unknown mechanism of action, activates orexin neurons (Mignot et al., Sleep, 11, 1012-1020, 1997; Chemelli et al., Cell, 98, 437-451, 1999). The intracerebroventricular (icv) administration of OX-A dose-dependently increases wakefulness in rat and also reduces total REM sleep by 84% (Piper et al., Eur. J. Neuroscience, 12, 726-730, 2000). Taken together, these observations are consistent with a crucial role of the orexin system in the modulation of sleep/wake cycle.

Orexin plays an important role in stress and anxiety via its interaction with the corticotropin-releasing factor (CRF) system in hypothalamus (Sakamoto et al., Regul Pept., 118, 183-91, 2004). The icv injection of OX-A induces grooming (stress-response) which is blocked in part by a CRF antagonist (Ida et al., Biochem. Biophys. Res. Comm., 270, 318-323, 2000). OX₂R is highly expressed in adrenal medulla, whereas OX₁R is high in adrenal cortex. Both OX-A and OX-B stimulate corticosterone release in plasma and induce c-Fos in paraventricular nucleus (PVN) in the hypothalamus (Kuru et al., Neuroreport, 11, 1977-1980, 2000). Furthermore, orexin neurons projecting to CRF neurons express mainly the OX₂R (Winsky-Sommerer et al., J. Neuroscience, 24, 11439-11448, 2004). Therefore, OX₂R stimulation activates the hypothalamo-pituitary-adrenal (HPA) axis. Interestingly, in this context, the orexin A-induced increases in plasma ACTH has been reported to be attenuated by a selective antagonist to OX-2R (N-{(1S)-1-(6,7-dimethoxy-3,4-dihydro-2(1H)-isoquinolinyl)carbonyl}-2,2-dimethylpropyl)-N-{4-pyridinylmethyl}amine (Chang et al., Neurosci Res., 21 Dec. 2006). A recent preclinical report (Suzuki et al., Brain Research, 1044, 116-121, 2005) has suggested an anxiogenic effect of OX-A. The icv injection of OX-A caused an anxiety-like behavior in mice. Effects were similar to those of corticotropin-releasing factor (CRF) that was tested at the same time for comparison. A recent study has also demonstrated the presence of functional OX1 and OX2 receptors in human adipose tissue and their roles in adipose tissue metabolism and adipogenesis (Digby et al., J. Endocrinol., 191, 129-36, 2006).

In summary, considering the very diverse functions played by orexin system in arousal, sleep/wakefulness, appetite regulation and their roles in anxiety and stress response, etc., one expects that the drugs (or compounds) targeting orexin system will have beneficial therapeutic effects for the treatments of diseases like sleep disorders including sleep apnea, narcolepsy, insomnia, parasomnia, jet lag syndrome, circadian rhythms disorder, restless leg syndrome, psychiatric, neurological and neurodegenerative disorders including anxiety, depression, manic depression, obsessive compulsive disorders, affective neurosis, depressive neurosis, anxiety neurosis, mood disorder, delirium, panic-attack disorder, posttraumatic stress disorders, sexual dysfunction, schizophrenia, psychosis, cognitive disorders, Alzheimer's and Parkinson's diseases, dementia, mental retardation, dyskinesias such as Huntington's disease and Tourette syndrome, addictions, craving associated with drug abuse, seizure disorders, epilepsy, metabolic diseases such as obesity, diabetes, eating disorders including anorexia and bulimia, asthma, migraine, pain, neuropathic pain, sleep disorders associated with psychiatric, neurological and neurodegenerative disorders, neuropathic pain, enhanced or exaggerated sensitivity to pain such as hyperalgesia, causalgia, and allodynia, acute pain, burn pain, back pain, complex regional pain syndrome I and II, arthritic pain, post-stroke pain, post-operative pain, neuralgia, pain associated with HIV infection, post-chemotherapy pain, irritable bowel syndrome and other diseases related to general orexin system dysfunction.

Numerous documents describe the current knowledge on orexin pathway, for example the following documents:
Expert Opin. Ther. Patents (2006), 16(5), 631-646
Current Opinion in Drug Discovery & Development, 2006, 9(5), 551-559
J. Neurosci (2000), 20(20), 7760-7765
Neurosci Lett, (2003), 341(3), 256-258

### SUMMARY OF THE INVENTION

The present invention provides compounds of formula

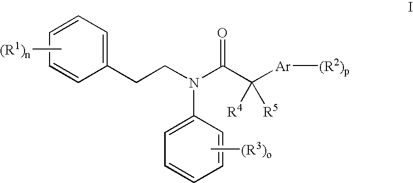

wherein
Ar is aryl or heteroaryl;
R¹ is hydrogen, halogen, lower alkyl, lower alkyl substituted by halogen, lower alkoxy, lower alkoxy substituted by halogen, cyano, SO₂-lower alkyl or hydroxy;
R² is hydrogen, halogen, lower alkyl, lower alkyl substituted by halogen, lower alkoxy, lower alkoxy substituted by halogen, cyano, S-lower alkyl, SO₂-lower alkyl, NO₂ or hydroxy;
R³ is hydrogen, halogen, lower alkyl, lower alkyl substituted by halogen, lower alkoxy, —(CH₂)ₘ—O-lower alkyl, lower alkoxy substituted by halogen, 3-hydroxy-oxetan-3-yl, cyano or SO₂-lower alkyl;
or if o is 2, R³ in the 3 and 4 positions together with the carbon atoms to which they are attached can form an additional ring with the groups —O—CH₂—O—, —O—CF₂—CF₂—O—, —N═CH—S—, —O—CF₂—O—, —(CH₂)₄—, —NH—C(O)—NH—, —O—(CH₂)₂— or —(CH₂)₂—O—;
R⁴ and R⁵ are each independently hydrogen, —(CR″₂)ₘOH, lower alkyl, lower alkoxy, —NRR′, or is —(CH₂)_{q}-heterocycloalkyl optionally substituted by hydroxy, or R⁴ and R⁵ together are ═O or ═N—OH;
R and R′ are each independently hydrogen, lower alkyl, C(O)H, —(CR″₂)ₘ—OH, —(CR″₂)ₘ—NR″₂, —(CR″₂)ₘ—NR″—C(O)-lower alkyl, —(CR″₂)ₘ—O-lower alkyl, —(CR″₂)ₘ—O-lower alkenyl, —C(O)O-lower alkyl, —C(O)—C(R″)₂—NH—C(O)O-lower alkyl, —C(O)—C(R″)₂—N(R″)₂, or is —(CH₂) q-heterocycloalkyl or —(CH₂) q-furan-2-yl;
each R″ is independently hydrogen, lower alkoxy, phenyl or lower alkyl;
n is 1, 2, 3 or 4;
o is 1, 2 or 3;
p is 1, 2 or 3;
m is 1, 2 or 3; and
q is 0 or 1;
   or to pharmaceutically suitable acid addition salts, optically pure enantiomers, racemates or diastereomeric mixtures thereof,
   with the exception of
N-(4-fluorophenyl)-3-hydroxy-N-[2-(3-methoxyphenyl)ethyl]benzeneacetamide (CAS=295319-21-0),
N-(4-fluorophenyl)-4-hydroxy-N-[2-(3-methoxyphenyl)ethyl]-3-methyl benzeneacetamide (CAS 295319-92-5),
4-bromo-N-[2-(3-methoxyphenyl)ethyl]-N-phenyl-benzeneacetamide (CAS 295318-80-8) and
N-(4-methoxyphenyl)-N-[2-(3-methoxyphenyl)ethyl]benzeneacetamide (CAS 436857-25-9).

The first three compounds are disclosed in U.S. Pat. No. 6,593,322, WO0224653 and WO0055137, filed by Signal Pharmaceuticals, as intermediates for the synthesis of modulators of estrogen receptors. The fourth compound has been described in WO0246164 by Astra Zeneca as an intermediate for the synthesis of selective estrogen receptor ligands.

The invention also provides pharmaceutical compositions containing a compound of formula I and a pharmaceutically acceptable salt thereof. The invention further provides methods for the manufacture of the compounds and compositions of the invention.

Compounds of formula I are orexin receptor antagonists and the related compounds can be useful in the treatment of disorders, in which orexin pathways are involved like sleep disorders including sleep apnea, narcolepsy, insomnia, parasomnia, jet lag syndrome, circadian rhythms disorder, restless leg syndrome, psychiatric, neurological and neurodegenerative disorders including anxiety, depression, manic depression, obsessive compulsive disorders, affective neurosis, depressive neurosis, anxiety neurosis, mood disorder, delirium, panic-attack disorder, posttraumatic stress disorders, sexual dysfunction, schizophrenia, psychosis, cognitive disorders, Alzheimer's and Parkinson's diseases, dementia, mental retardation, dyskinesias such as Huntington's disease and Tourette syndrome, addictions, craving associated with drug abuse, seizure disorders, epilepsy, metabolic diseases such as obesity, diabetes, eating disorders including anorexia and bulimia, asthma, migraine, pain, neuropathic pain, sleep disorders associated with psychiatric, neurological and neurodegenerative disorders, neuropathic pain, enhanced or exaggerated sensitivity to pain such as hyperalgesia, causalgia, and allodynia, acute pain, burn pain, back pain, complex regional pain syndrome I and II, arthritic pain, post-stroke pain, post-operative pain, neuralgia, pain associated with HIV infection, post-chemotherapy pain, irritable bowel syndrome and other diseases related to general orexin system dysfunction.

### DETAILED DESCRIPTION OF THE INVENTION

The following definitions of the general terms used in the present description apply irrespective of whether the terms in question appear alone or in combination.

As used herein, the term “lower alkyl” denotes a straight- or branched-chain hydrocarbon group containing from 1-4 carbon atoms, for example, methyl, ethyl, propyl, isopropyl, n-butyl, i-butyl, t-butyl and the like. The term “alkyl” denotes a straight- or branched-chain alkyl group containing from 1-7 carbon atoms.

The term “lower alkoxy” denotes an alkyl group as defined above, which is attached via an oxygen atom.

The term “halogen” denotes chlorine, iodine, fluorine and bromine.

The term “cycloalkyl” denotes a saturated carbocyclic ring having from 3 to 7 ring carbon atoms. Examples of cycloalkyl include cyclopentyl, cyclobutyl, cyclopentyl, cyclohexyl, and cycloheptyl.

The term “aryl” means the monovalent cyclic aromatic hydrocarbon group consisting of one or more fused rings in which at least one ring is aromatic in nature. Examples of aryl radicals include, but are not limited to, phenyl, naphthyl, biphenyl, indanyl, anthraquinolyl, and the like.

“Heteroaryl” means the monovalent cyclic aromatic group having one or more rings incorporating one, two, or three heteroatoms within the ring (chosen from nitrogen, oxygen, or sulfur). Examples of heteroaryl radicals include, but are not limited to, imidazolyl, imidazo[4,5-b]pyridin-1-yl, oxazolyl, 1,3-benzodioxol-5-yl, isoxazolyl, thiazolyl, thiophen-2 or 3-yl, yl, furanyl, pyridin-2, 3 or 4-yl, pyrazinyl, benzoimidazol-1 or 2-yl, pyrimidinyl, pyridazinyl, quinolinyl, isoquinolinyl, benzofuryl, benzothiophenyl, benzothiopyranyl, benzooxazolyl, benzothiazolyl, benzopyranyl, indazol-1-yl, indolyl, isoindolyl, purin-7 or 9-yl, naphthyridinyl, and the like.

The term “heterocycloalkyl” means a saturated monovalent ring system, wherein one or more ring atom is a N, O or S. Examples for such heterocycloalkyl groups are for example pyrrolidin 1-yl, imidazolidinyl, pyrazolidinyl, piperidin-1-yl, 2-oxa-6-aza-spiro[3.3]hept-6-yl, piperazinyl, dioxolan-2-yl, tetrahydrofuran-2-yl, morpholinyl, thiomorpholinyl, azetidinyl-yl, azepanyl and the like.

As used herein, the term “lower alkyl substituted by halogen” denotes an alkyl group as defined above, wherein at least one hydrogen atom is replaced by halogen, for example CF₃, CHF₂, CH₂F, CH₂CF₃, CH₂CH₂CF₃, CH₂CF₂CF₃ and the like.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

“Therapeutically effective amount” means an amount that is effective to prevent, alleviate or ameliorate symptoms of disease or prolong the survival of the subject being treated.

Preferred compounds of formula I are those wherein Ar is phenyl.

Preferred compounds from this group are those, wherein one of R⁴/R⁵ is hydroxy, for example the following compounds
N-(3,4-dimethoxyphenyl)-2-hydroxy-2-(2-methoxyphenyl)-N-{2-[4-(trifluoromethyl)phenyl]ethyl}acetamide,
(2S)—N-(3,4-dimethoxyphenyl)-2-hydroxy-2-phenyl-N-{2-[4-(trifluoromethyl)phenyl]ethyl}acetamide,
N-(3,4-dimethoxyphenyl)-2-(4-fluorophenyl)-2-hydroxy-N-{2-[4-(trifluoromethyl)phenyl]ethyl}acetamide,
N-(3,4-dimethoxyphenyl)-2-hydroxy-2-(3-hydroxy-4-methoxyphenyl)-N-{2-[4-(trifluoromethyl)phenyl]ethyl}acetamide,
2-(4-chlorophenyl)-N-(3,4-dimethoxyphenyl)-2-hydroxy-N-{2-[4-(trifluoromethyl)phenyl]ethyl}acetamide,
N-(3,4-dimethoxyphenyl)-2-(2-fluorophenyl)-2-hydroxy-N-{2-[4-(trifluoromethyl)phenyl]ethyl}acetamide,
(S)—N-(3,4-dimethyl-phenyl)-2-hydroxy-2-phenyl-N-[2-(4-trifluoromethyl-phenyl)-ethyl]-acetamide, and
(S)—N-(3,4-dimethyl-phenyl)-2-(4-fluoro-phenyl)-2-hydroxy-N-[2-(4-trifluoromethyl-phenyl)-ethyl]-acetamide.

Preferred compounds from this group are further those, wherein both of R⁴/R⁵ are hydrogen, for example the following compound
N-(3,4-dimethoxyphenyl)-2-(2-methoxyphenyl)-N-{2-[4-(trifluoromethyl)phenyl]ethyl}acetamide.

Preferred compounds from this group are those, wherein one of R⁴/R⁵ is NH₂, for example the following compounds
2-amino-N-(3,4-dimethoxy-phenyl)-2-(2-methoxy-phenyl)-N-[2-(4-trifluoromethyl-phenyl)-ethyl]-acetamide,
2-amino-2-(2-methoxy-phenyl)-N-(3-methoxy-phenyl)-N-[2-(4-trifluoromethyl-phenyl)-ethyl]-acetamide,
(2-amino-N-benzo[1,3]dioxol-5-yl-2-(2-methoxy-phenyl)-N-[2-(4-trifluoromethyl-phenyl)-ethyl]-acetamide,
(2-amino-N-(3-fluoro-4-methoxy-phenyl)-2-(2-methoxy-phenyl)-N-[2-(4-trifluoromethyl-phenyl)-ethyl]-acetamide,
2-amino-N-(3-chloro-4-methoxy-phenyl)-2-(2-methoxy-phenyl)-N-[2-(4-trifluoromethyl-phenyl)-ethyl]-acetamide,
2-amino-2-(2-methoxy-phenyl)-N-(2,2,3,3-tetrafluoro-2,3-dihydro-benzo[1,4]dioxin-6-yl)-N-[2-(4-trifluoromethyl-phenyl)-ethyl]-acetamide,
2-amino-N-(3,4-dimethoxy-phenyl)-2-phenyl-N-[2-(4-trifluoromethyl-phenyl)-ethyl]-acetamide,
2-amino-N-benzo[1,3]dioxol-5-yl-2-phenyl-N-[2-(4-trifluoromethyl-phenyl)-ethyl]-acetamide,
2-amino-N-(5-methoxy-2-methyl-phenyl)-2-phenyl-N-[2-(4-trifluoromethyl-phenyl)-ethyl]-acetamide,
2-amino-N-(3-methoxy-phenyl)-2-phenyl-N-[2-(4-trifluoromethyl-phenyl)-ethyl]-acetamide,
2-amino-N-(3,4-dimethoxy-phenyl)-2-(4-fluoro-phenyl)-N-[2-(4-trifluoromethyl-phenyl)-ethyl]-acetamide,
2-amino-N-(3,4-dimethoxy-phenyl)-2-(4-fluoro-phenyl)-N-[2-(4-trifluoromethyl-phenyl)-ethyl]-acetamide,
2-amino-2-(3-chloro-phenyl)-N-(3,4-dimethoxy-phenyl)-N-[2-(4-trifluoromethyl-phenyl)-ethyl]-acetamide,
2-amino-N-(3-fluoro-4-methoxy-phenyl)-2-phenyl-N-[2-(4-trifluoromethyl-phenyl)-ethyl]-acetamide,
2-amino-N-(4-chloro-3-methoxy-phenyl)-2-phenyl-N-[2-(4-trifluoromethyl-phenyl)-ethyl]-acetamide,
2-amino-N-(3-chloro-4-methoxy-phenyl)-2-phenyl-N-[2-(4-trifluoromethyl-phenyl)-ethyl]-acetamide,
2-amino-N-(3,4-diethoxy-phenyl)-2-phenyl-N-[2-(4-trifluoromethyl-phenyl)-ethyl]-acetamide,
2-amino-2-phenyl-N-p-tolyl-N-[2-(4-trifluoromethyl-phenyl)-ethyl]-acetamide,
2-amino-2-phenyl-N-m-tolyl-N-[2-(4-trifluoromethyl-phenyl)-ethyl]-acetamide,
2-amino-N-(4-methoxy-phenyl)-2-phenyl-N-[2-(4-trifluoromethyl-phenyl)-ethyl]-acetamide,
2-amino-N-[2-(3,4-dimethoxy-phenyl)-ethyl]-2-phenyl-N-(4-trifluoromethoxy-phenyl)-acetamide,
2-amino-N-(4-fluoro-3-methyl-phenyl)-2-phenyl-N-[2-(4-trifluoromethyl-phenyl)-ethyl]-acetamide,
2-amino-N-(3,4-dimethyl-phenyl)-2-phenyl-N-[2-(4-trifluoromethyl-phenyl)-ethyl]-acetamide,
2-amino-N-(3,4-dimethoxy-phenyl)-2-(3-fluoro-phenyl)-N-[2-(4-trifluoromethyl-phenyl)-ethyl]-acetamide,
2-amino-N-(4-fluoro-3-methyl-phenyl)-2-phenyl-N-(2-p-tolyl-ethyl)-acetamide,
2-amino-N-(3,4-dimethyl-phenyl)-2-phenyl-N-(2-p-tolyl-ethyl)-acetamide,
2-amino-N-[2-(4-chloro-phenyl)-ethyl]-N-(3,4-dimethyl-phenyl)-2-phenyl-acetamide,
2-amino-N-[3-(3-hydroxy-oxetan-3-yl)-phenyl]-2-phenyl-N-[2-(4-trifluoromethyl-phenyl)-ethyl]-acetamide,
(S)-2-amino-N-(3,4-dimethoxy-phenyl)-2-(2-methoxy-phenyl)-N-[2-(4-trifluoromethyl-phenyl)-ethyl]-acetamide,
2-amino-N-(4-fluoro-3-methoxy-phenyl)-2-phenyl-N-[2-(4-trifluoromethyl-phenyl)-ethyl]-acetamide,
2-amino-N-(4-difluoromethoxy-phenyl)-2-phenyl-N-[2-(4-trifluoromethyl-phenyl)-ethyl]-acetamide,
2-amino-N-(2-chloro-5-methoxy-phenyl)-2-phenyl-N-[2-(4-trifluoromethyl-phenyl)-ethyl]-acetamide,
2-amino-2-phenyl-N-(5,6,7,8-tetrahydro-naphthalen-2-yl)-N-[2-(4-trifluoromethyl-phenyl)-ethyl]-acetamide,
(S)-2-amino-N-(3,4-dimethyl-phenyl)-2-phenyl-N-[2-(4-trifluoromethyl-phenyl)-ethyl]-acetamide,
(S)-2-amino-N-(3,4-diethoxy-phenyl)-2-phenyl-N-[2-(4-trifluoromethyl-phenyl)-ethyl]-acetamide,
(S)-2-amino-N-(3,4-dimethyl-phenyl)-2-(4-fluoro-phenyl)-N-(2-p-tolyl-ethyl)-acetamide,
(S)-2-amino-2-phenyl-N-p-tolyl-N-[2-(4-trifluoromethyl-phenyl)-ethyl]-acetamide,
(S)-2-amino-N-(4-difluoromethoxy-phenyl)-2-phenyl-N-[2-(4-trifluoromethyl-phenyl)-ethyl]-acetamide,
(S)-2-amino-2-phenyl-N-(5,6,7,8-tetrahydro-naphthalen-2-yl)-N-[2-(4-trifluoromethyl-phenyl)-ethyl]-acetamide,
(S)-2-amino-N-(3-methoxy-phenyl)-2-phenyl-N-[2-(4-trifluoromethyl-phenyl)-ethyl]-acetamide,
(S)-2-amino-N-(3,4-dimethyl-phenyl)-2-(4-fluoro-phenyl)-N-[2-(4-trifluoromethyl-phenyl)-ethyl]-acetamide,
(S)-2-amino-N-(3-methoxy-4-methyl-phenyl)-2-phenyl-N-[2-(4-trifluoromethyl-phenyl)-ethyl]-acetamide,
(S)-2-amino-N-(3-chloro-4-difluoromethoxy-phenyl)-2-(4-fluoro-phenyl)-N-[2-(4-trifluoromethyl-phenyl)-ethyl]-acetamide,
(S)-2-amino-N-(3-chloro-4-ethoxy-phenyl)-2-(4-fluoro-phenyl)-N-[2-(4-trifluoromethyl-phenyl)-ethyl]-acetamide,
(S)-2-amino-N-(3,4-dimethoxy-phenyl)-2-phenyl-N-[2-(4-trifluoromethyl-phenyl)-ethyl]-acetamide hydrochloride,
(R)-2-amino-N-(3,4-dimethoxy-phenyl)-2-phenyl-N-[2-(4-trifluoromethyl-phenyl)-ethyl]-acetamide,
(S)-2-amino-N-(3,4-dimethyl-phenyl)-N-[2-(4-fluoro-phenyl)-ethyl]-2-phenyl-acetamide,
(S)-2-amino-N-(3,4-dimethyl-phenyl)-2-phenyl-N-[2-(4-trifluoromethoxy-phenyl)-ethyl]-acetamide,
(S)-2-amino-N-(3,4-dimethyl-phenyl)-N-[2-(2-fluoro-phenyl)-ethyl]-2-phenyl-acetamide,
(S)-2-amino-N-(3,4-dimethyl-phenyl)-2-phenyl-N-(2-m-tolyl-ethyl)-acetamide,
(S)-2-amino-N-(3,4-dimethyl-phenyl)-N-[2-(3-fluoro-phenyl)-ethyl]-2-phenyl-acetamide,
(S)-2-amino-N-[2-(3-chloro-phenyl)-ethyl]-N-(3,4-dimethyl-phenyl)-2-phenyl-acetamide,
(S)-2-amino-N-(3,4-dimethyl-phenyl)-2-phenyl-N-[2-(3-trifluoromethyl-phenyl)-ethyl]-acetamide,
(S)-2-amino-N-(3,4-dimethyl-phenyl)-2-phenyl-N-(2-o-tolyl-ethyl)-acetamide,
(S)-2-amino-N-(3,4-dimethyl-phenyl)-N-[2-(3-fluoro-4-trifluoromethyl-phenyl)-ethyl]-2-phenyl-acetamide,
(S)-2-amino-N-(3,4-dimethyl-phenyl)-N-[2-(4-fluoro-3-trifluoromethyl-phenyl)-ethyl]-2-phenyl-acetamide,
(S)-2-amino-N-[2-(2,4-difluoro-phenyl)-ethyl]-N-(3,4-dimethyl-phenyl)-2-phenyl-acetamide,
(S)-2-amino-N-[2-(3,4-difluoro-phenyl)-ethyl]-N-(3,4-dimethyl-phenyl)-2-phenyl-acetamide,
(S)-2-amino-N-(3,4-dimethyl-phenyl)-N-[2-(3-fluoro-4-methyl-phenyl)-ethyl]-2-phenyl-acetamide,
(S)-2-amino-N-[2-(2,3-difluoro-phenyl)-ethyl]-N-(3,4-dimethyl-phenyl)-2-phenyl-acetamide,
(S)-2-amino-N-[2-(4-chloro-2-fluoro-phenyl)-ethyl]-N-(3,4-dimethyl-phenyl)-2-phenyl-acetamide,
(S)-2-amino-N-(3,4-dimethyl-phenyl)-N-[2-(2-fluoro-5-trifluoromethyl-phenyl)-ethyl]-2-phenyl-acetamide,
(S)-2-amino-N-(3,4-dimethyl-phenyl)-N-[2-(2-methoxy-phenyl)-ethyl]-2-phenyl-acetamide,
(S)-2-amino-N-(3,4-dimethyl-phenyl)-N-[2-(2-fluoro-3-trifluoromethyl-phenyl)-ethyl]-2-phenyl-acetamide,
(S)-2-amino-N-(3,4-dimethyl-phenyl)-N-[2-(2-fluoro-4-trifluoromethyl-phenyl)-ethyl]-2-phenyl-acetamide,
(S)-2-amino-N-[2-(2,3-difluoro-4-trifluoromethyl-phenyl)-ethyl]-N-(3,4-dimethyl-phenyl)-2-phenyl-acetamide,
(S)-2-amino-N-(3,4-dimethyl-phenyl)-2-phenyl-N-[2-(2-trifluoromethoxy-phenyl)-ethyl]-acetamide,
(S)-2-amino-N-(3,4-dimethyl-phenyl)-N-phenethyl-2-phenyl-acetamide,
(S)-2-amino-N-[2-(4-chloro-3-fluoro-phenyl)-ethyl]-N-(3,4-dimethyl-phenyl)-2-phenyl-acetamide,
(S)-2-amino-N-(3,4-dimethyl-phenyl)-N-[2-(3-fluoro-5-trifluoromethyl-phenyl)-ethyl]-2-phenyl-acetamide,
(S)-2-amino-N-(3,4-dimethyl-phenyl)-2-phenyl-N-[2-(2,3,6-trifluoro-4-trifluoromethyl-phenyl)-ethyl]-acetamide,
(S)-2-amino-N-[2-(2,5-dichloro-phenyl)-ethyl]-N-(3,4-dimethyl-phenyl)-2-phenyl-acetamide,
(S)-2-amino-N-[2-(3-chloro-2-fluoro-phenyl)-ethyl]-N-(3,4-dimethyl-phenyl)-2-phenyl-acetamide,
(S)-2-amino-N-[2-(2,4-dichloro-phenyl)-ethyl]-N-(3,4-dimethyl-phenyl)-2-phenyl-acetamide,
(S)-2-amino-N-(3,4-dimethyl-phenyl)-N-[2-(3-hydroxy-phenyl)-ethyl]-2-phenyl-acetamide,
(S)-2-amino-N-[2-(3,5-difluoro-phenyl)-ethyl]-N-(3,4-dimethyl-phenyl)-2-phenyl-acetamide,
(S)-2-amino-N-[2-(3-chloro-5-fluoro-phenyl)-ethyl]-N-(3,4-dimethyl-phenyl)-2-phenyl-acetamide,
(S)-2-amino-N-[2-(4-difluoromethoxy-phenyl)-ethyl]-N-(3,4-dimethyl-phenyl)-2-phenyl-acetamide,
(S)-2-amino-N-[2-(4-cyano-phenyl)-ethyl]-N-(3,4-dimethyl-phenyl)-2-phenyl-acetamide,
(S)-2-amino-N-(2,3-dihydro-benzofuran-5-yl)-N-[2-(4-fluoro-phenyl)-ethyl]-2-phenyl-acetamide,
(S)-2-amino-N-(2,3-dihydro-benzofuran-6-yl)-N-[2-(4-fluoro-phenyl)-ethyl]-2-phenyl-acetamide, and
(S)-2-amino-N-(4-ethyl-phenyl)-N-[2-(4-fluoro-phenyl)-ethyl]-2-phenyl-acetamide.

Preferred compounds from this group are those, wherein one of R⁴/R⁵ is NRR′ and R/R′ is other than hydrogen, for example the following compounds
N-(3,4-dimethoxy-phenyl)-2-(2-methoxy-phenyl)-2-methylamino-N-[2-(4-trifluoromethyl-phenyl)-ethyl]-acetamide,
N-(3,4-dimethoxy-phenyl)-2-methylamino-2-phenyl-N-[2-(4-trifluoromethyl-phenyl)-ethyl]-acetamide,
(S)—N-(3,4-dimethoxy-phenyl)-2-(oxetan-3-ylamino)-2-phenyl-N-[2-(4-trifluoromethyl-phenyl)-ethyl]-acetamide,
(S)—N-(3,4-dimethoxy-phenyl)-2-(2-methoxy-phenyl)-2-methylamino-N-[2-(4-trifluoromethyl-phenyl)-ethyl]-acetamide,
N-(3,4-dimethyl-phenyl)-2-(2-hydroxy-ethylamino)-2-phenyl-N-(2-p-tolyl-ethyl)-acetamide,
N-(3,4-dimethyl-phenyl)-2-(2-methoxy-1-methyl-ethylamino)-2-phenyl-N-(2-p-tolyl-ethyl)-acetamide,
N-(3,4-dimethyl-phenyl)-2-(2-hydroxy-propylamino)-2-phenyl-N-(2-p-tolyl-ethyl)-acetamide,
N-(3,4-dimethyl-phenyl)-2-(2-hydroxy-1-methyl-ethylamino)-2-phenyl-N-(2-p-tolyl-ethyl)-acetamide,
N-(3,4-dimethyl-phenyl)-2-(1-hydroxymethyl-2-methyl-propylamino)-2-phenyl-N-(2-p-tolyl-ethyl)-acetamide,
N-(3,4-dimethyl-phenyl)-2-(1-methoxymethyl-propylamino)-2-phenyl-N-(2-p-tolyl-ethyl)-acetamide,
2-(2-acetylamino-ethylamino)-N-(3,4-dimethyl-phenyl)-2-phenyl-N-(2-p-tolyl-ethyl)-acetamide,
N-(3,4-dimethyl-phenyl)-2-phenyl-2-[(tetrahydro-furan-2-ylmethyl)-amino]-N-(2-p-tolyl-ethyl)-acetamide,
2-(2,2-dimethoxy-ethylamino)-N-(3,4-dimethyl-phenyl)-2-phenyl-N-(2-p-tolyl-ethyl)-acetamide,
N-(3,4-dimethyl-phenyl)-2-[(furan-2-ylmethyl)-amino]-2-phenyl-N-(2-p-tolyl-ethyl)-acetamide,
N-(3,4-dimethyl-phenyl)-2-(2-hydroxy-butylamino)-2-phenyl-N-(2-p-tolyl-ethyl)-acetamide,
N-(3,4-dimethyl-phenyl)-2-(2-hydroxy-1,1-dimethyl-ethylamino)-2-phenyl-N-(2-p-tolyl-ethyl)-acetamide,
N-(3,4-dimethyl-phenyl)-2-[([1,3]dioxolan-2-ylmethyl)-amino]-2-phenyl-N-(2-p-tolyl-ethyl)-acetamide,
N-(3,4-dimethyl-phenyl)-2-phenyl-2-{[(S)-1-(tetrahydro-furan-2-yl)methyl]-amino}-N-(2-p-tolyl-ethyl)-acetamide,
N-(3,4-dimethyl-phenyl)-2-phenyl-N-(2-p-tolyl-ethyl)-2-(2-vinyloxy-ethylamino)-acetamide,
N-(3,4-dimethyl-phenyl)-2-(2-ethoxy-ethylamino)-2-phenyl-N-(2-p-tolyl-ethyl)-acetamide,
(S)—N-(3,4-dimethyl-phenyl)-2-(2-methoxy-phenyl)-2-methylamino-N-[2-(4-trifluoromethyl-phenyl)-ethyl]-acetamide,
(R,S)—N-(3,4-dimethyl-phenyl)-2-ethoxy-2-phenyl-N-[2-(4-trifluoromethyl-phenyl)-ethyl]-acetamide,
(S)-2-(2-amino-acetylamino)-N-(3,4-dimethyl-phenyl)-2-phenyl-N-(2-p-tolyl-ethyl)-acetamide,
(S)-2-((S)-2-amino-2-phenyl-acetylamino)-N-(3,4-dimethyl-phenyl)-2-phenyl-N-(2-p-tolyl-ethyl)-acetamide, and
(S)-2-amino-N-{(S)-[(3,4-dimethyl-phenyl)-(2-p-tolyl-ethyl)-carbamoyl]-phenyl-methyl}-propionamide.

Preferred compounds from this group are those, wherein R⁴ and R⁵ are together ═O or ═N—OH, for example the following compounds
N-(3,4-dimethyl-phenyl)-2-[hydroxyimino]-2-phenyl-N-[2-(2,3,6-trifluoro-4-trifluoromethyl-phenyl)-ethyl]-acetamide and
N-(3,4-dimethyl-phenyl)-2-oxo-2-phenyl-N-[2-(4-trifluoromethyl-phenyl)-ethyl]-acetamide.
   Preferred compounds of formula I are those wherein Ar is heteroaryl, for example the following compounds
2-amino-2-(5-chloro-thiophen-2-yl)-N-[2-(3,4-difluoro-phenyl)-ethyl]-N-(3,4-dimethyl-phenyl)-acetamide hydrochloride,
2-amino-N-[2-(3,4-difluoro-phenyl)-ethyl]-N-(3,4-dimethyl-phenyl)-2-thiophen-3-yl-acetamide hydrochloride, and
N-(3,4-dimethyl-phenyl)-2-(2-methyl-benzoimidazol-1-yl)-N-[2-(4-trifluoromethyl-phenyl)-ethyl]-acetamide.

A further object of the present invention are compounds of formula

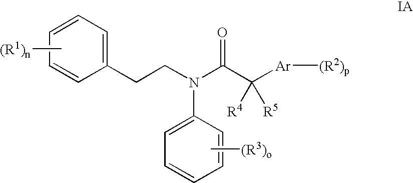

wherein
Ar is aryl or heteroaryl;
R¹ is hydrogen, halogen, lower alkyl, lower alkyl substituted by halogen, lower alkoxy, lower alkoxy substituted by halogen, cyano, SO₂-lower alkyl, cycloalkyl or heterocycloalkyl;
R² is hydrogen, halogen, lower alkyl, lower alkyl substituted by halogen, lower alkoxy, lower alkoxy substituted by halogen, cyano, S-lower alkyl, SO₂-lower alkyl, cycloalkyl, heterocycloalkyl, NO₂ or hydroxy;
R³ is hydrogen, halogen, lower alkyl, lower alkyl substituted by halogen, lower alkoxy, —(CH₂)ₘ—O-lower alkyl, lower alkoxy substituted by halogen, cyano, SO₂-lower alkyl, cycloalkyl, or heterocycloalkyl;
R⁴ and R⁵ are each independently hydrogen, hydroxy, lower alkyl, lower alkoxy, —NRR′ or R⁴ and R⁵ are together ═O;
R and R′ are each independently hydrogen, lower alkyl, cycloalkyl, hydroxy, —(CH₂)ₘ—OH, —(CH₂)ₘ—O-lower alkyl or heterocycloalkyl,
   or together with the N atom to which they are attached form a heterocycloalkyl ring, optionally containing in addition to the N atom a further heteroatom, selected from the group consisting of N, S and O,
n is 1, 2 or 3;
o is 1, 2 or 3;
p is 1, 2 or 3; and
m is 1, 2 or 3;
   wherein all cycloalkyl- or heterocycloalkyl groups as defined for R¹, R², R³ are unsubstituted or substituted by one or more substituents selected from the group consisting of hydroxy, halogen, lower alkyl and lower alkoxy;
   with the exception of
N-(4-fluorophenyl)-3-hydroxy-N-[2-(3-methoxyphenyl)ethyl]benzeneacetamide (CAS=295319-21-0),
N-(4-fluorophenyl)-4-hydroxy-N-[2-(3-methoxyphenyl)ethyl]-3-methyl benzeneacetamide (CAS 295319-92-5),
4-bromo-N-[2-(3-methoxyphenyl)ethyl]-N-phenyl-benzeneacetamide (CAS 295318-80-8) and
N-(4-methoxyphenyl)-N-[2-(3-methoxyphenyl)ethyl]benzeneacetamide (CAS 436857-25-9).

The present compounds of formula I and their pharmaceutically acceptable salts can be prepared by methods known in the art, for example, by processes described below, which process comprises

a) reacting a compound of formula

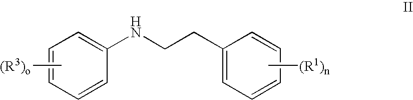

with a compound of formula

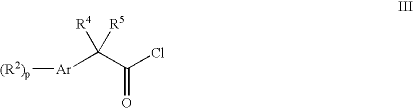

to give the compound of formula

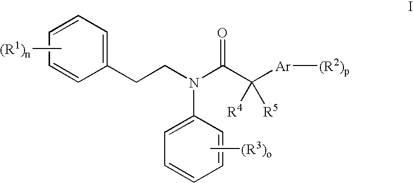

wherein the substituents are as described above, or
b) reacting a compound of formula

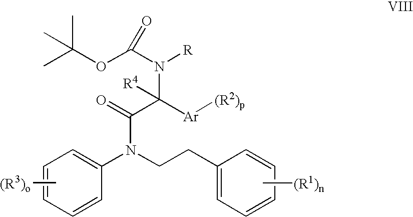

with a compound of formula

R′I

to give the compound of formula

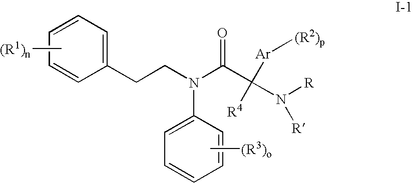

wherein the substituents are as described above, and
and

if desired, converting the compounds obtained into pharmaceutically acceptable acid addition salts.

The preparation of compounds of formula I of the present invention can be carried out in sequential or convergent synthetic routes. Syntheses of the compounds of the invention are shown in the following scheme. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in scheme 1, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

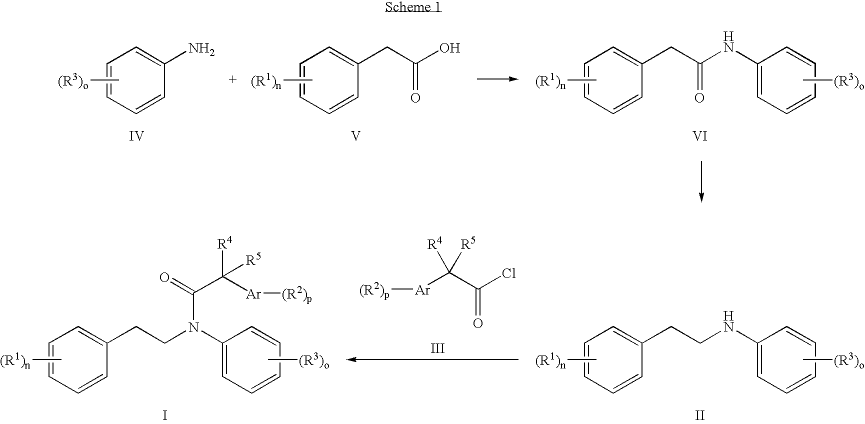

Phenyl amine derivatives IV and benzylacetic acid derivatives V are commercially available or can be accessed by methods described in literature. Reaction of phenyl amine derivatives IV with benzylacetic acid derivatives V can be achieved by various methods as described in literature (for reaction conditions described in literature affecting such reactions see for example: Comprehensive Organic Transformations: A Guide to Functional Group Preparations, 2^{nd} Edition, Richard C. Larock. John Wiley & Sons, New York, N.Y. 1999). However, it is convenient to react phenyl amine derivative IV with benzyl acetic acid derivative V in the presence of a coupling reagent, a base and a solvent. For example coupling reagents like N,N′-carbonyl diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) and the like can equally well be employed to affect such transformation. We find it convenient to carry out the reaction in a solvent like dimethylformamide (DMF) and in the presence of a base. There is no particular restriction on the nature of the solvent to be employed, provided that it has no adverse effect on the reaction or the reagents involved and that it can dissolve the reagents, at least to some extent. Examples for suitable solvents include: DMF, dichloromethane (DCM), dioxane, THF, and the like. There is no particular restriction on the nature of the base used in this stage, and any base commonly used in this type of reaction may equally be employed here. Examples of such bases include triethylamine and diisopropylethylamine, and the like. The reaction can take place over a wide range of temperatures, and the precise reaction temperature is not critical to the invention. We find it convenient to carry out the reaction with heating from ambient temperature to reflux. The time required for the reaction can also vary widely, depending on many factors, notably the reaction temperature and the nature of the reagents. However, a period of from 0.5 h to several days will usually suffice to yield amide derivatives VI.

a) Reduction of the amide derivatives VI to the corresponding amine derivatives II can be achieved by various methods as described in literature. However, it is convenient to react amide derivative VI with a reducing agent in the presence of a solvent. For example lithium aluminium hydride (LiAlH₄) or borane (BH₃) and the like can equally well be employed to affect such transformation. We find it convenient to carry out the reaction in a solvent like tetrahydrofuran (THF). There is no particular restriction on the nature of the solvent to be employed, provided that it has no adverse effect on the reaction or the reagents involved and that it can dissolve the reagents, at least to some extent. The reaction can take place over a wide range of temperatures, and the precise reaction temperature is not critical to the invention. We find it convenient to carry out the reaction with heating from ambient temperature to reflux. The time required for the reaction can also vary widely, depending on many factors, notably the reaction temperature and the nature of the reagents. However, a period of from 0.5 h to several days will usually suffice to yield amine derivatives II.

b) Amine derivatives II can be reacted with Aryl-acetic acid derivatives III to form amide derivatives I under various conditions. For reaction conditions described in literature affecting such or similar reactions see for example: Comprehensive Organic Transformations: A Guide to Functional Group Preparations, 2^{nd} Edition, Richard C. Larock. John Wiley & Sons, New York, N.Y. 1999). Aryl-acetic acid derivatives are either commercially available or can be prepared from commercially available starting materials. It is convenient to react amine derivative II with aryl-acetic acid derivatives III pre-activated through transformation into the respective acid chloride, or by employing a coupling reagent during the course of the reaction. This can be done in a solvent in the presence of a base. For example coupling reagents like N,N′-carbonyldiimidazole (CDI), N,N′-dicyclohexylcarbodiimide (DCC), 1-(3-dimethylaminopropyl)-3-ethylcarbodiimide hydrochloride (EDCI), 1-[bis(dimethylamino)methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) and the like can equally well be employed to affect such transformation. We find it convenient to carry out the reaction in a solvent like dimethylformamide (DMF) or dichloromethane (DCM) and in the presence of a base. There is no particular restriction on the nature of the solvent to be employed, provided that it has no adverse effect on the reaction or the reagents involved and that it can dissolve the reagents, at least to some extent. Examples for other suitable solvents include: dioxane, THF, and the like. There is no particular restriction on the nature of the base used in this stage, and any base commonly used in this type of reaction may equally be employed here. Examples of such bases include triethylamine and diisopropylethylamine, and the like. The reaction can take place over a wide range of temperatures, and the precise reaction temperature is not critical to the invention. We find it convenient to carry out the reaction with heating from ambient temperature to reflux. The time required for the reaction can also vary widely, depending on many factors, notably the reaction temperature and the nature of the reagents. However, a period of from 0.5 h to several days will usually suffice to yield amide derivatives I.

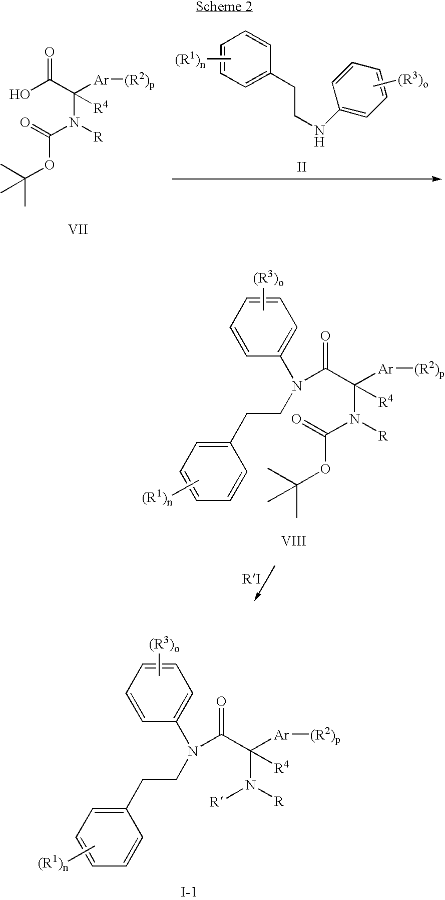

A compound of formula VII can be prepared, for example as follows:

A mixture of a substituted Aryl-amino-acetic acid, di-tert-butyl dicarbonate and N,N-diisopropyl ethyl amine in DCM is stirred at room temperature for about 15 h. All volatiles are removed under reduced pressure, and the residue is taken up in EtOAc and citric acid. The organic phase is dried and evaporated to dryness.

Then a mixture of II, a compound of formula VII, HATU and NEt₃ in DMF is stirred at 80° C. for about 15 h. All volatiles are removed under reduced pressure and the residue is taken up in EtOAc and NaHCO₃. The organic phase is dried and evaporated to dryness.

Then a mixture of a compound of formula VIII, sodium hydride and R′I is stirred at RT for about 15 h. All volatiles were removed under reduced pressure, and the residue is taken up in DCM and trifluoroacetic acid. The mixture is stirred for about 5 h at RT. All volatiles are removed under reduced pressure, and the residue is taken up in EtOAc and NaHCO₃. The organic phase is dried and evaporated to dryness to obtain a compound of formula I-1.

The compounds were investigated in accordance with the test given hereinafter.

### Intracellular Ca²⁺ Mobilization Assay

The Chinese Hamster Ovary (dHFr−) mutant cell line stably expressing human orexin-1 (hOX1) or human orexin-2 (hOX2) receptors were maintained in Dulbecco's Modified Eagle Medium (1×) with GlutaMax™ 1, 4500 mg/L D-Glucose and Sodium Pyruvate (Catalog No. 31966-021, Invitrogen, Carlsbad, Calif.), 5% dialyzed fetal calf serum (Catalog No. 26400-044), 100 μg/ml penicillin and 100 μg/ml streptomycin. The cells were seeded at 5×10⁴ cells/well in the poly-D-lysine treated, 96-well, black/clear-bottomed plates (Catalog No. BD356640, BD Biosciences, Palo Alto, Calif.). 24 h later, the cells were loaded for 1 h at 37° C. with 4 μM Flou-4 acetoxymethyl ester (Catalog No. F-14202, Molecular Probes, Eugene, Oreg.) in FLIPR buffer (1×HBSS, 20 mM HEPES, 2.5 mM Probenecid). Hanks' Balanced Salt Solution (HBSS) (10×) (catalog No. 14065-049) and HEPES (1M) (catalog No. 15630-056) were purchased from Invitrogen, Carlsbad, Calif. Probenecid (250 mM) (catalog No. P8761) was from Sigma, Buchs, Switzerland. The cells were washed five times with FLIPR buffer to remove excess dye and intracellular calcium mobilization, [Ca²⁺]ᵢ were measured using a Fluorometric Imaging Plate Reader (FLIPR-96, Molecular Devices, Menlo Park, Calif.) as described previously (Malherbe et al., Mol. Pharmacol., 64, 823-832, 2003). Orexin A (catalog No. 1455, Toris Cookson Ltd, Bristol, UK) was used as agonist. Orexin A (50 mM stock solution in DMSO) was diluted in FLIPR buffer +0.1% BSA. The EC₅₀ and EC₈₀ values of orexin-A were measured daily from standard agonist concentration-response curves in CHO(dHFr−) —OX1R and —OX2R cell lines. All compounds were dissolved in 100% DMSO. Inhibition curves were determined by addition of 11 concentrations (0.0001-10 μM) of inhibitory compounds and using EC₈₀ value of orexin-A as agonist (a concentration which gave 80% of max agonist response, determined daily). The antagonists were applied 25 min (incubation at 37° C.) before the application of the agonist. Responses were measured as peak increase in fluorescence minus basal, normalized to the maximal stimulatory effect induced by EC₈₀ value of orexin-A or orexin-B. Inhibition curves were fitted according to the Hill equation: y=100/(1+(x/IC₅₀)^{nH}), where n_{H}=slope factor using Excel-fit 4 software (Microsoft).

K_{b} values were calculated according to the following equation K_{b}=IC₅₀/(1+[A]/EC₅₀) where A is the concentration of agonist added which is very close to agonist EC₈₀ value, and IC₅₀ and EC50 values were derived from the antagonist inhibition and orexin-A or B agonist curves, respectively.

The preferred compounds show a K_{b} value (μM)<0.1 in human on orexin receptor.

Representative compounds are shown in the table below.

| | K_{b} (μM) |
|---|---|
| | OX2R |
| Example | (human) |
| 14 | 0.0342 |
| 17 | 0.0165 |
| 23 | 0.0226 |
| 26 | 0.0592 |
| 27 | 0.0737 |
| 30 | 0.0757 |
| 31 | 0.0336 |
| 34 | 0.027 |
| 35 | 0.0088 |
| 38 | 0.0577 |
| 41 | 0.0505 |
| 42 | 0.0559 |
| 43 | 0.0162 |
| 44 | 0.0069 |
| 45 | 0.0018 |
| 53 | 0.0305 |
| 58 | 0.0275 |
| 59 | 0.0143 |
| 60 | 0.0231 |
| 61 | 0.0025 |
| 62 | 0.0751 |
| 63 | 0.0664 |
| 64 | 0.0383 |
| 65 | 0.0285 |
| 66 | 0.013 |
| 68 | 0.0117 |
| 69 | 0.0828 |
| 70 | 0.0579 |
| 72 | 0.0023 |
| 75 | 0.0523 |
| 78 | 0.0444 |
| 83 | 0.0698 |
| 84 | 0.0666 |
| 85 | 0.0074 |
| 88 | 0.0819 |
| 89 | 0.0241 |
| 93 | 0.0888 |
| 95 | 0.0834 |
| 101 | 0.0133 |
| 103 | 0.0053 |
| 104 | 0.022 |
| 105 | 0.0414 |
| 106 | 0.0071 |
| 107 | 0.0509 |
| 110 | 0.0335 |
| 120 | 0.0446 |
| 121 | 0.0109 |
| 122 | 0.0225 |
| 123 | 0.0727 |
| 125 | 0.0267 |
| 126 | 0.0086 |
| 127 | 0.0222 |
| 129 | 0.0173 |
| 130 | 0.0416 |
| 131 | 0.022 |
| 132 | 0.0642 |
| 133 | 0.007 |
| 134 | 0.0161 |
| 135 | 0.0379 |
| 136 | 0.0201 |
| 137 | 0.0364 |
| 138 | 0.0364 |
| 140 | 0.0127 |
| 144 | 0.0174 |
| 146 | 0.0335 |
| 148 | 0.0217 |
| 150 | 0.0682 |
| 151 | 0.0112 |
| 154 | 0.051 |
| 157 | 0.03218 |
| 159 | 0.0542 |
| 160 | 0.0291 |
| 161 | 0.0908 |
| 162 | 0.0151 |
| 163 | 0.0778 |
| 165 | 0.0673 |
| 173 | 0.0011 |
| 174 | 0.046 |
| 175 | 0.0219 |
| 176 | 0.0505 |
| 180 | 0.036 |
| 181 | 0.0708 |
| 182 | 0.0283 |
| 183 | 0.0496 |
| 184 | 0.0249 |
| 185 | 0.0305 |
| 186 | 0.0156 |
| 187 | 0.0327 |
| 188 | 0.0269 |
| 189 | 0.0071 |
| 190 | 0.0091 |
| 191 | 0.0215 |
| 192 | 0.0148 |
| 193 | 0.016 |
| 194 | 0.0052 |
| 195 | 0.0176 |
| 196 | 0.0171 |
| 197 | 0.0067 |
| 199 | 0.0489 |
| 200 | 0.0143 |
| 201 | 0.247 |
| 202 | 0.0015 |
| 203 | 0.0996 |
| 204 | 0.0178 |
| 205 | 0.0829 |
| 206 | 0.0469 |
| 208 | 0.0231 |
| 209 | 0.0486 |
| 210 | 0.0077 |
| 211 | 0.0222 |
| 212 | 0.0343 |
| 214 | 0.0966 |
| 216 | 0.0302 |
| 218 | 0.0555 |
| 221 | 0.0834 |
| 236 | 0.076 |
| 239 | 0.025 |
| 243 | 0.0604 |
| 244 | 0.049 |
| 248 | 0.0353 |
| 249 | 0.082 |
| 251 | 0.0102 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The present invention also provides a method for the manufacture of pharmaceutical compositions. Such process comprises bringing one or more compounds of formula I and/or pharmaceutically acceptable acid addition salts thereof and, if desired, one or more other therapeutically valuable substances into a galenical administration form together with one or more therapeutically inert carriers.

The most preferred indications in accordance with the present invention are those, which include sleep disorders including sleep apnea, narcolepsy, insomnia, parasomnia, jet lag syndrome, circadian rhythms disorder, restless leg syndrome, psychiatric, neurological and neurodegenerative disorders including anxiety, depression, manic depression, obsessive compulsive disorders, affective neurosis, depressive neurosis, anxiety neurosis, mood disorder, delirium, panic-attack disorder, posttraumatic stress disorders, sexual dysfunction, schizophrenia, psychosis, cognitive disorders, Alzheimer's and Parkinson's diseases, dementia, mental retardation, dyskinesias such as Huntington's disease and Tourette syndrome, addictions, craving associated with drug abuse, seizure disorders, epilepsy, metabolic diseases such as obesity, diabetes, eating disorders including anorexia and bulimia, asthma, migraine, pain, neuropathic pain, sleep disorders associated with psychiatric, neurological and neurodegenerative disorders, neuropathic pain, enhanced or exaggerated sensitivity to pain such as hyperalgesia, causalgia, and allodynia, acute pain, burn pain, back pain, complex regional pain syndrome I and II, arthritic pain, post-stroke pain, post-operative pain, neuralgia, pain associated with HIV infection, post-chemotherapy pain, irritable bowel syndrome and other diseases related to general orexin system dysfunction.

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

### EXAMPLE 1

### N-(3,4-Dimethoxy-phenyl)-2-phenyl-N-[2-(4-trifluoromethyl-phenyl)-ethyl]-acetamide

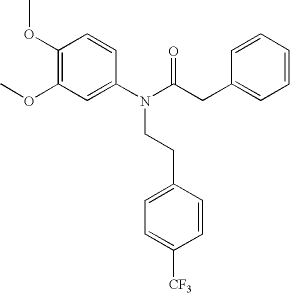

### a) Step 1:

### N-(3,4-Dimethoxy-phenyl)-2-(4-trifluoromethyl-phenyl)-acetamide

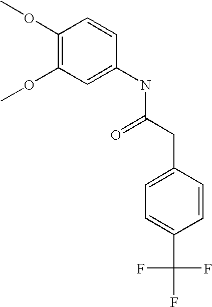

A mixture of 4.00 g (26 mmol) 3,4-dimethoxy-phenylamine (commercially available), 5.88 g (29 mmol) (4-trifluoro-phenyl)-acetic acid (commercially available), 10.00 g (31 mmol) TBTU and 5.28 g (52 mmol) NEt₃ in 15 mL DMF was stirred at room temperature for 30 minutes. All volatiles were removed under reduced pressure and the residue was taken up in DCM and 1M aq. HCl. The organic phase was dried with MgSO₄ and evaporated to dryness. The residue was triturated with DCM and ethyl acetate to yield after drying 7.88 g (89%) of the title compound. MS (m/e): 340.3 (MH⁺).

### b) Step 2:

### (3,4-Dimethoxy-phenyl)-[2-(4-trifluoromethyl-phenyl)-ethyl]-amine

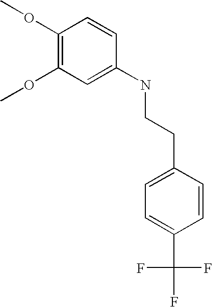

A mixture of 3.00 g (8.8 mmol) N-(3,4-Dimethoxy-phenyl)-2-(4-trifluoromethyl-phenyl)-acetamide and 1.00 g (26.3 mmol) LiAlH₄ in 100 mL THF was stirred for 1 h at room temperature. Water and HCl aq. was added and the mixture was extracted with ethyl acetate. The combined organic phases were washed with water, dried with MgSO₄ and evaporated to dryness. The residue was purified by flash column chromatography on silica eluting with a gradient formed from ethyl acetate and heptane. The product containing fractions were combined and evaporated to dryness to yield 0.70 g (24%) of the title compound. MS (m/e): 326.1 (MH⁺).

### c) Step 3:

### N-(3,4-Dimethoxy-phenyl)-2-phenyl-N-[2-(4-trifluoromethyl-phenyl)-ethyl]-acetamide

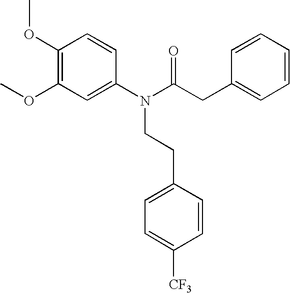

A mixture of 32.5 mg (0.1 mmol) (3,4-Dimethoxy-phenyl)-[2-(4-trifluoromethyl-phenyl)-ethyl]-amine, 23.2 mg (0.15 mmol) phenylacetyl chloride and 30.3 mg (0.3 mmol) NEt₃ in 2 mL DCM was stirred at room temperature for 16 h. The mixture was concentrated and re-dissolved in methanol/acetic acid and subjected to purification by preparative HPLC on reversed phase eluting with a gradient formed from acetonitrile, water, acetic acid. The product containing fractions were evaporated to yield 17.6 mg (40%) of the title compound. MS (m/e): 444.1 (MH⁺).

In analogy to the procedure described for the synthesis of examples 1 further amide derivatives have been synthesized from their respective starting materials mentioned in table 1. The examples are shown in table 1 and comprise example 2-example 33:

TABLE 1

| | | | | | MW |
|---|---|---|---|---|---|
| | | | | | found |
| No. | Structure | MW | Name | Starting Materials | (MH+) |
| 2 | 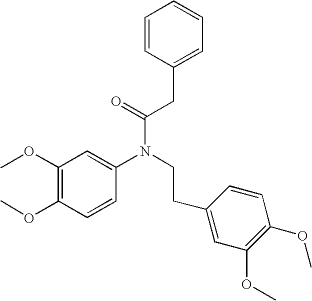 | 435.5 | N-(3,4-dimethoxyphenyl)-N-[2-(3,4-dimethoxyphenyl)ethyl]-2-phenylacetamide | (3,4-Dimethoxy-phenyl)-[2-(3,4-dimethoxy-phenyl)-ethyl]-amine andPhenyl-acetic acid(commerciallyavailable) | 436.2 |
| 3 | 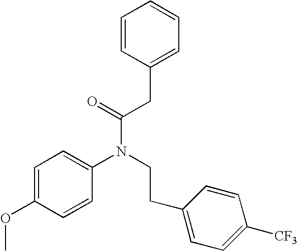 | 413.4 | N-(4-methoxyphenyl)-2-phenyl-N-{2-[4-(trifluoromethyl)phenyl]ethyl}acetamide | (4-Methoxy-phenyl)-[2-(4-trifluoromethyl-phenyl)-ethyl]-amine and Phenyl-acetic acid(commerciallyavailable) | 414.1 |
| 4 | 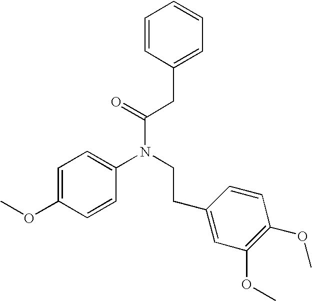 | 405.5 | N-[2-(3,4-dimethoxyphenyl)ethyl]-N-(4-methoxyphenyl)-2-phenylacetamide | [2-(3,4-Dimethoxy-phenyl)-ethyl]-(4-methoxy-phenyl)-amine and Phenyl-acetic acid(commerciallyavailable) | 406.2 |
| 5 | 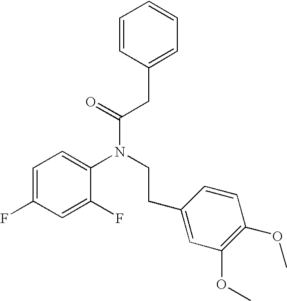 | 411.4 | N-(2,4-difluorophenyl)-N-[2-(3,4-dimethoxyphenyl)ethyl]-2-phenylacetamide | 4-[2-(2,4-Difluoro-phenylamino)-ethyl]-2-methoxy-phenol and Phenyl-acetic acid(commerciallyavailable) | 412.1 |
| 6 | 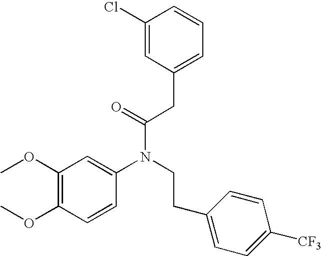 | 477.9 | 2-(3-chlorophenyl)-N-(3,4-dimethoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine and (3-Chloro-phenyl)-acetic acid(commerciallyavailable) | 478.2 |
| 7 | 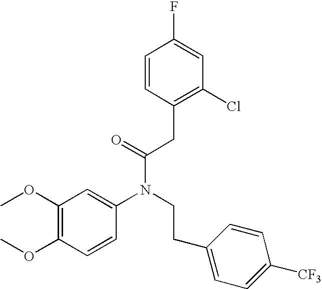 | 495.9 | 2-(2-chloro-4-fluorophenyl)-N-(3,4-dimethoxyphenyl)-N-{2-[4-(trifluoromethyl-phenyl)-ethyl}acetamide | (3,4-Dimethoxy-phenyl)-[2-(4-trifluoromethyl-phenyl]-amine and (2-Chloro-4-fluoro-phenyl)-acetic acid(commerciallyavailable) | 496.2 |
| 8 | 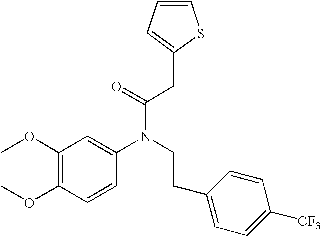 | 449.5 | N-(3,4-dimethoxyphenyl)-2-(2-thienyl)-N-{2-[4-(trifluoromethyl)phenyl]ethyl}acetamide | (3,4-Dimethoxy-phenyl)-[2-(4-trifluoromethyl-phenyl)-ethyl]-amine andThiophen-2-yl-acticacid (commerciallyavailable) | 450.2 |
| 9 | 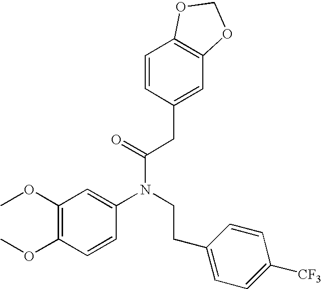 | 487.5 | 2-(1,3-benzodioxol-5-yl)-N-(3,4-dimethoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine andBenzo[1,3]dioxol-5-yl-acetic acid(commerciallyavailable) | 488.2 |
| 10 | 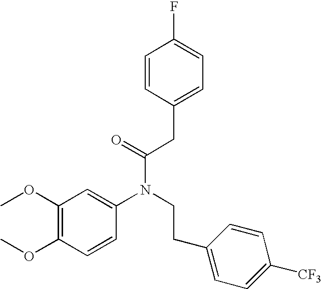 | 461.5 | N-(3,4-dimethoxyphenyl)-2-(4-fluorophenyl)-N-{2-[4-(trifluoromethyl)phenyl]ethyl}acetamide | (3,4-Dimethoxy-phenyl)-[2-(4-trifluoromethyl-phenyl)-ethyl]-amine and (4-Fluoro-phenyl)-acetic acid(commerciallyavailable | 462.2 |
| 11 | 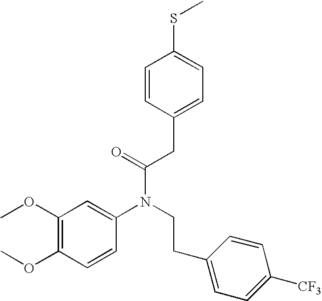 | 489.6 | N-(3,4-dimethoxyphenyl)-2-[4-(methylthio)phenyl]-N-{2-[4-(trifluoromethyl)phenyl]ethyl}acetamide | (3,4-Dimethoxy-phenyl)-[2-(4-trifluoromethyl-phenyl)-ethyl]-amine and (4-Methylsulfanyl-phenyl)-acetic acid(commerciallyavailable) | 490.2 |
| 12 | 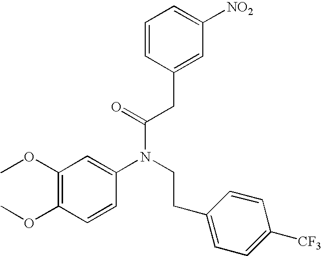 | 488.5 | N-(3,4-dimethoxyphenyl)-2-(3-nitrophenyl)-N-{2-[4-(trifluoromethyl)phenyl]ethyl}acetamide | (3,4-Dimethoxy-phenyl)-[2-(4-trifluoromethyl-phenyl)-ethyl]-amine and (3-Nitro-phenyl)-aceticacid (commerciallyavailable) | 489.2 |
| 13 | 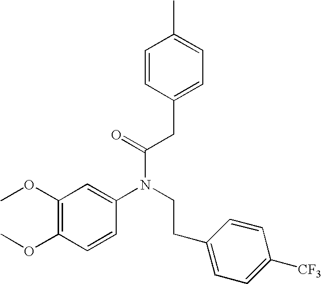 | 457.5 | N-(3,4-dimethoxyphenyl)-2-(4-methylphenyl)-N-{2-[4-(trifluoromethyl)phenyl]ethyl}acetamide | (3,4-Dimethoxy-phenyl)-[2-(4-trifluoromethyl-phenyl)-ethyl]-amine and p-Tolyl-acetic acid(commerciallyavailable | 458.2 |
| 14 | 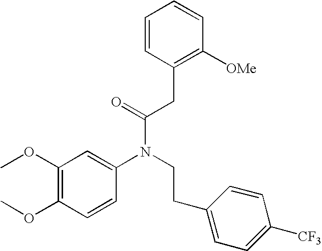 | 473.5 | N-(3,4-dimethoxyphenyl)-2-(2-methoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine and (2-Methoxy-phenyl)-acetic acid(commerciallyavailable | 474.2 |
| 15 | 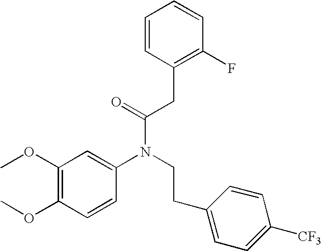 | 461.5 | N-(3,4-dimethoxyphenyl)-2-(2-fluorophenyl)-N-{2-[4-(trifluoromethyl)phenyl]ethyl}acetamide | (3,4-Dimethoxy-phenyl)-[2-(4-trifluoromethyl-phenyl)-ethyl]-amine and (2-Fluoro-phenyl)-acetic acid(commerciallyavailable | 462.2 |
| 16 | 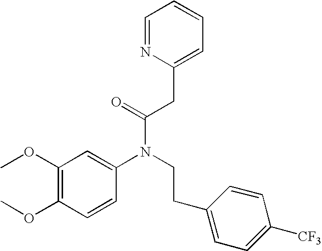 | 444.5 | #N!-(3,4-Dimethoxy-phenyl)-2-pyridin-2-yl-#N!-[2-(4-trifluoromethyl-phenyl)-ethyl]-acetamide | (3,4-Dimethoxy-phenyl)-[2-(4-trifluoromethyl-phenyl)-ethyl]-amine and Pyridin-2-yl-aceticacid (commerciallyavailable) | 445.2 |
| 17 | 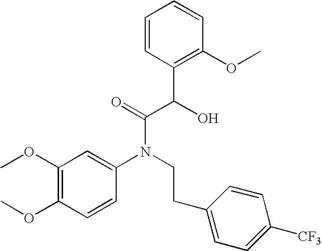 | 489.5 | N-(3,4-dimethoxyphenyl)-2-hydroxy-2-(2-methoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine andHydroxy-(2-methoxy-phenyl)-acetic acid(commerciallyavailable) | 490.2 |
| 18 | 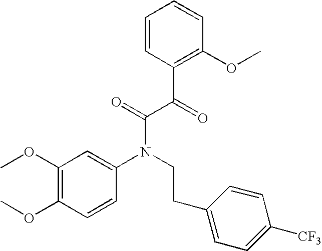 | 487.5 | N-(3,4-dimethoxyphenyl)-2-(2-methoxyphenyl)-2-oxo-N-{2-[4-(trifluoromethyl)phenyl]ethyl}acetamide | (3,4-Dimethoxy-phenyl)-[2-(4-trifluoromethyl-phenyl)-ethyl]-amine and (2-Methoxy-phenyl)-oxo-acetic acid(commerciallyavailable) | 488.2 |
| 19 | 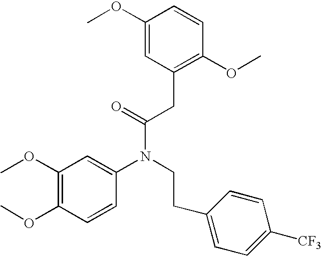 | 503.5 | 2-(2,5-dimethoxyphenyl)-N-(3,4-dimethoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine and(2,5-Dimethoxy-phenyl)-acetic acid(commerciallyavailable) | 504.2 |
| 20 | 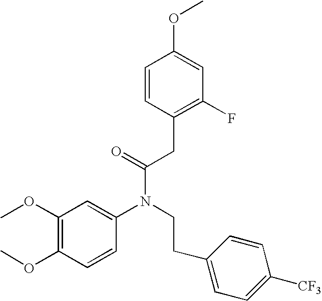 | 491.5 | N-(3,4-dimethoxyphenyl)-2-(2-fluoro-4-methoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine and (2-Fluoro-4-methoxy-phenyl)-aceticacid (commerciallyavailable) | 492.2 |
| 21 | 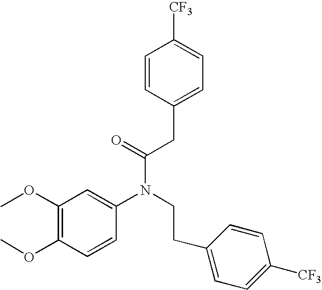 | 511.5 | N-(3,4-dimethoxyphenyl)-2-[4-(trifluoromethyl)phenyl]-N-{2-[4-(trifluoromethyl)phenyl]ethyl}acetamide | (3,4-Dimethoxy-phenyl)-[2-(4-trifluoromethyl-phenyl)-ethyl]-amine (4-Trifluoromethyl-phenyl)-acetic acid(commerciallyavailable) | 512.2 |
| 22 | 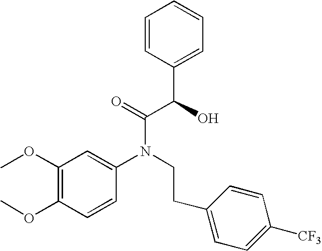 | 459.5 | (2R)-N-(3,4-dimethoxyphenyl)-2-hydroxy-2-phenyl-N-{2-[4-(trifluoromethyl)phenyl]ethyl}acetamide | (3,4-Dimethoxy-phenyl)-[2-(4-trifluoromethyl-phenyl)-ethyl]-amine and(R)-Hydroxy-phenyl-acetic acid(commerciallyavailable) | 460.2 |
| 23 | 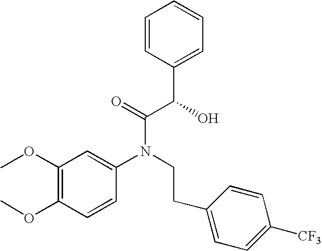 | 459.5 | (2S)-N-(3,4-dimethoxyphenyl)-2-hydroxy-2-phenyl-N-{2-[4-(trifluoromethyl)phenyl]ethyl}acetamide | (3,4-Dimethoxy-phenyl)-[2-(4-trifluoromethyl-phenyl)-ethyl]-amine and(S)-Hydroxy-phenyl-acetic acid(commerciallyavailable) | 460.2 |
| 24 | 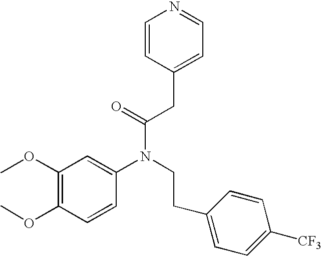 | 444.5 | N-(3,4-dimethoxyphenyl)-2-pyridin-4-yl-N-{2-[4-(trifluoromethyl)phenyl]ethyl}acetamide | (3,4-Dimethoxy-phenyl)-[2-(4-trifluoromethyl-phenyl)-ethyl]-amine andPyridin-4-yl-aceticacid (commerciallyavailable) | 445.2 |
| 25 | 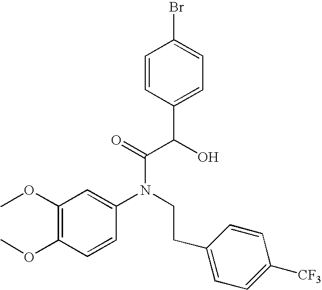 | 538.4 | 2-(4-bromophenyl)-N-(3,4-dimethoxyphenyl)-2-hydroxy-N-{2-[4-(trifluoromethyl)phenyl]ethyl}acetamide | (3,4-Dimethoxy-phenyl)-[2-(4-trifluoromethyl-phenyl)-ethyl]-amine and (4-Bromo-phenyl)-hydroxy-acetic acid(commerciallyavailable | 538.1 |
| 26 | 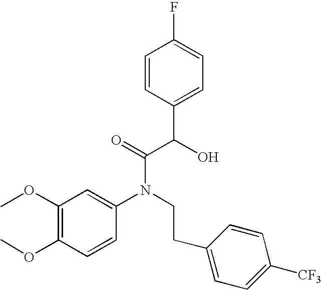 | 477.5 | N-(3,4-dimethoxyphenyl)-2-(4-fluorophenyl)-2-hydroxy-N-{2-[4-(trifluoromethyl)phenyl]ethyl}acetamide | (3,4-Dimethoxy-phenyl)-[2-(4-trifluoromethyl-phenyl)-ethyl]-amine and (4-Fluoro-phenyl)-hydroxy-acetic acidm(commerciallyavailable) | 478.2 |
| 27 | 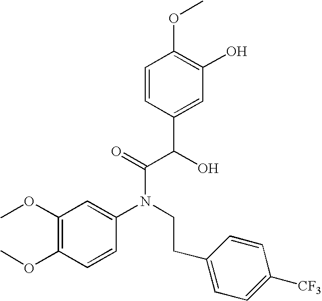 | 505.5 | N-(3,4-dimethoxyphenyl)-2-hydroxy-2-(3-hydroxy-4-methoxyphenyl)-N-{2-[4-(trifluoromethyl)phenyl]ethyl}acetamide | (3,4-Dimethoxy-phenyl)-[2-(4-trifluoromethyl-phenyl)-ethyl]-amine andHydroxy-(3-hydroxy-4-methoxy-phenyl)-aceticacid (commerciallyavailable) | 506.2 |
| 28 | 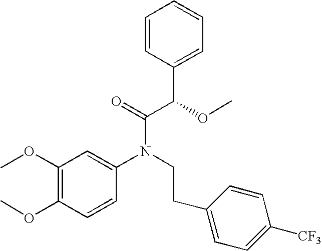 | 473.5 | (2S)-N-(3,4-dimethoxyphenyl)-2-methoxy-2-phenyl-N-{2-[4-(trifluoromethyl)phenyl]ethyl}acetamide | (3,4-Dimethoxy-phenyl)-[2-(4-trifluoromethyl-phenyl)-ethyl]-amine and(S)-Methoxy-phenyl-acetic acid(commerciallyavailable) | 474.2 |
| 29 | 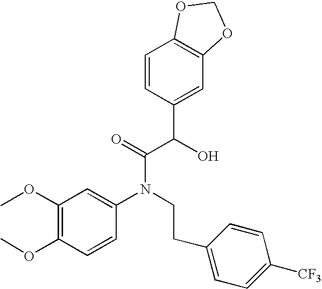 | 503.5 | 2-(1,3-benzodioxol-5-yl)-N-(3,4-dimethoxyphenyl)-2-hydroxy-N-{2-[4-(trifluoromethyl)phenyl]ethyl}acetamide | (3,4-Dimethoxy-phenyl)-[2-(4-trifluoromethyl-phenyl)-ethyl]-amine andBenzo[1,3]dioxol-5-yl-hydroxy-acetic acid(commerciallyavailable) | 504.2 |
| 30 | 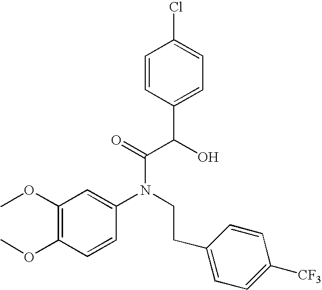 | 493.9 | 2-(4-chlorophenyl)-N-(3,4-dimethoxyphenyl)-2-hydroxy-N-{2-[4-(trifluoromethyl)phenyl]ethyl}acetamide | (3,4-Dimethoxy-phenyl)-[2-(4-trifluoromethyl-phenyl)-ethyl]-amine and (4-Chloro-phenyl)-hydroxy-acetic acid(commerciallyavailable) | 494.2 |
| 31 | 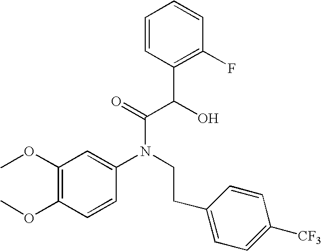 | 477.5 | N-(3,4-dimethoxyphenyl)-2-(2-fluorophenyl)-2-hydroxy-N-{2-[4-(trifluoromethyl)phenyl]ethyl}acetamide | (3,4-Dimethoxy-phenyl)-[2-(4-trifluoromethyl-phenyl)-ethyl]-amine and (2-Fluoro-phenyl)-hydroxy-acetic acid(commerciallyavailable) | 478.1 |
| 32 | 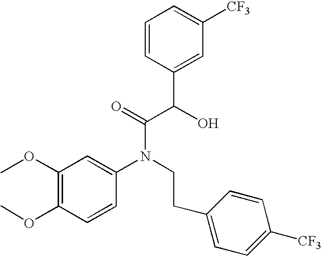 | 527.5 | N-(3,4-dimethoxyphenyl)-2-hydroxy-2-[3-(trifluoromethyl)phenyl]-N-{2-[4-(trifluoromethyl)phenyl]ethyl}acetamide | (3,4-Dimethoxy-phenyl)-[2-(4-trifluoromethyl-phenyl)-ethyl]-amine and Hydroxy-(3-trifluoromethyl-phenyl)-acetic acid(commerciallyavailable) | 528.2 |
| 33 | 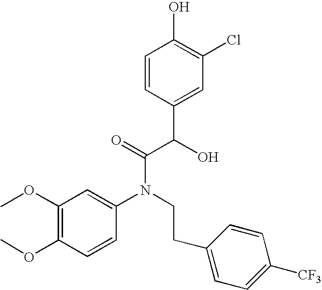 | 509.9 | 2-(3-chloro-4-hydroxyphenyl)-N-(3,4-dimethoxyphenyl)-2-hydroxy-N-{2-[4-(trifluromethyl)phenyl]ethyl}acetamide | (3,4-Dimethoxy-phenyl)-[2-(4-trifluoromethyl-phenyl)-ethyl]-amine and (3-Chloro-4-hydroxy-phenyl)-hydroxy-acetic acid(described in EP23459 19810204) | 510.2 |

### EXAMPLE 34

### N-(3,4-Dimethoxy-phenyl)-2-(2-methoxy-phenyl)-2-methylamino-N-[2-(4-trifluoromethyl-phenyl)-ethyl]-acetamide

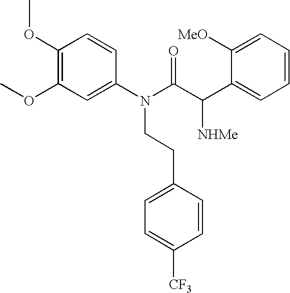

### Step 1:

### tert-Butoxycarbonylamino-(2-methoxy-phenyl)-acetic acid

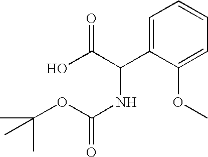

A mixture of 500 mg (2.8 mmol) amino-(2-methoxy-phenyl)-acetic acid, 602 mg (2.8 mmol) di-tert-butyl dicarbonate and 357 mg (2.8 mmol) N,N-diisopropyl ethyl amine in 25 mL DCM was stirred at room temperature for 15 h. All volatiles were removed under reduced pressure and the residue was taken up in EtOAc and 10% aq. citric acid. The organic phase was dried with MgSO₄ and evaporated to dryness. The residue yielded after drying 731 mg (94%) of the title compound. MS (m/e): 280.1 (M−H⁺) and was used crude for the next step.

### Step 2:

### [{(3,4-Dimethoxy-phenyl)-[2-(4-trifluoromethyl-phenyl)-ethyl]-carbamoyl}-(2methoxyphenyl)-methyl]-carbamic acid tert-butyl ester

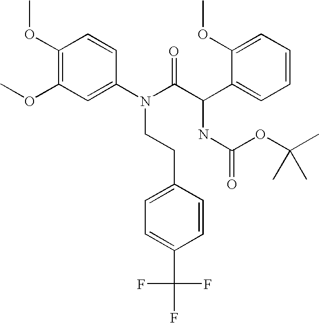

A mixture of 90 mg (0.32 mmol) tert-butoxycarbonylamino-(2-methoxy-phenyl)-acetic acid, 109 mg (0.33 mmol) (3,4-dimethoxy-phenyl)-[2-(4-trifluoromethyl-phenyl)-ethyl]-amine, 128 mg (0.33 mmol) HATU and 43 mg (0.33 mmol) NEt₃ in 3 mL DMF was stirred at 80° C. for 15 h. All volatiles were removed under reduced pressure and the residue was taken up in EtOAc and 1N NaHCO₃. The organic phase was dried with MgSO₄ and evaporated to dryness. The residue was purified by flash column chromatography on silica eluting with a gradient formed from ethyl acetate and heptane to yield after drying 60 mg (32%) of the title compound. MS (m/e): 589.3 (MH⁺).

### Step 3:

### N-(3,4-Dimethoxy-phenyl)-2-(2-methoxy-phenyl)-2-methylamino-N-[2-(4-trifluoromethyl-phenyl)-ethyl]-acetamide

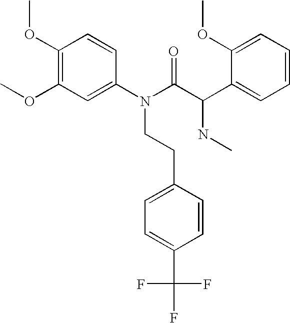

A mixture of 30 mg (0.05 mmol) [{(3,4-dimethoxy-phenyl)-[2-(4-trifluoromethyl-phenyl)-ethyl]-carbamoyl}-(2-methoxy-phenyl)-methyl]-carbamic acid tert-butyl ester, 3 mg (0.055 mmol) sodium hydride, 8 mg (0.055 mmol) MeI in 1 mL DMF was stirred at RT for 15 h. All volatiles were removed under reduced pressure and the residue was taken up in DCM and 58 mg (0.55 mmol) trifluoroacetic acid. The mixture was stirred for 5 h at RT All volatiles were removed under reduced pressure and the residue was taken up in EtOAc and 1M aq. NaHCO₃. The organic phase was dried with MgSO₄ and evaporated to dryness. The residue was purified by preparative HPLC to yield after drying 15 mg (59%) of the title compound. MS (m/e): 503.1 (MH⁺).

### EXAMPLE 35

### 2-Amino-N-(3,4-dimethoxy-phenyl)-2-(2-methoxy-phenyl)-N-[2-(4-trifluoromethyl-phenyl)-ethyl]-acetamide

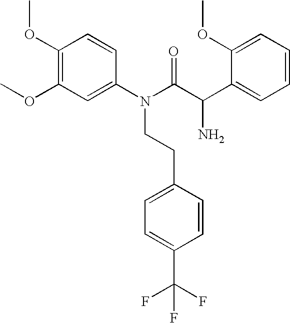

A mixture of 20 mg (0.034 mmol) [{(3,4-dimethoxy-phenyl)-[2-(4-trifluoromethyl-phenyl)-ethyl]-carbamoyl}-(2-methoxy-phenyl)-methyl]-carbamic acid tert-butyl ester (prepared herein) was dissolved in DCM and 39 mg (0.34 mmol) trifluoroacetic acid. The mixture was stirred for 5 h at RT All volatiles were removed under reduced pressure and the residue was taken up in EtOAc and 1M aq. NaHCO₃. The organic phase was dried with MgSO₄ and evaporated to dryness. The residue was purified by preparative HPLC to yield after drying 5 mg (30%) of the title compound. MS (m/e): 489.3 (MH⁺).

### EXAMPLE 36

### 2-(2-Methoxy-phenyl)-N-m-tolyl-N-[2-(4-trifluoromethyl-phenyl)-ethyl]-acetamide

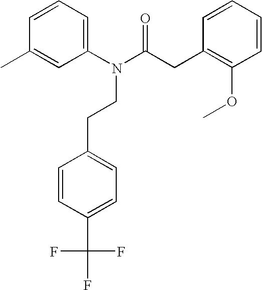

### a) Step 1:

### m-Tolyl-[2-(4-trifluoromethyl-phenyl)-ethyl]-amine

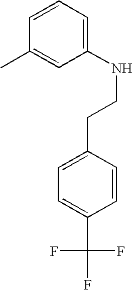

To a solution of m-toluidine (0.434 g, 4.1 mmol) and (4-trifluoromethyl)-phenylacetic acid (0.5 g, 0.25 mmol) in dichloromethane (8 mL) were added at rt TBTU (0.865 g, 2.6 mmol) and N,N-diisopropyl ethyl amine (0.348 g, 2.6 mmol). The resulting reaction mixture was stirred at this temperature under Argon for 12 h, then a solution of borane-tetrahydrofuran complex (1 M in THF, 3.67 ml, 4 mmol) was added and the reaction mixture was heated at 50° C. for 48 h before quenching with the addition of aqueous HCl (1 M, 1 mL). After cooling to ambient temperature it was diluted with water (2 mL) and basified with sodium carbonate. Extraction with diethylether was followed by washing with aqueous NaHCO₃ (saturated) and brine. Drying over sodium sulfate afforded the title compound (0.517 g, 76%) as a colourless oil. MS m/e: 280.1 [M+H]⁺.

### b) Step 2:

### 2-(2-Methoxy-phenyl)-N-m-tolyl-N-[2-(4-trifluoromethyl-phenyl)-ethyl]-acetamide

To a solution of m-tolyl-[2-(4-trifluoromethyl-phenyl)-ethyl]-amine (0.517 g, 1.9 mmol) and 2-methoxyphenylacetic acid (0.308 g, 1.85 mmol) in dichloromethane (8 mL) was added TBTU (0.654 g, 2.0 mmol) and N-ethyldiisopropylamine (0.35 mL, 0.2 mmol) at rt. After stirring for 4 d, the reaction mixture was washed aqueous HCl (1 M, 3×10 mL), aqueous sodium carbonate (half-saturated, 3×10 ml) and H₂0 (3×10 mL). The aqueous layers were washed with dichloromethane (20 mL). The combined organic layers were dried over sodium sulfate. Concentration and purification by chromatography (SiO₂, heptane:ethyl acetate=2:1) afforded the title compound (0.053 g, 7%) as a light-yellow oil. MS m/e: 428.3 [M+H]⁺.

### EXAMPLE 37

### 2 (S)—N-(3,4-Dimethoxy-phenyl)-2-hydroxy-2-(2-methoxy-phenyl)-N-[2-(4-trifluoromethyl-phenyl)-ethyl]-acetamide

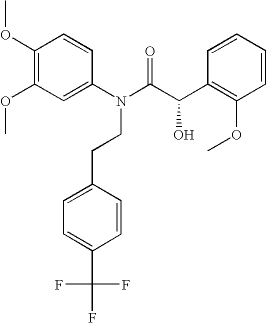

### a) Step 1:

### N-(3,4-Dimethoxy-phenyl)-2-(4-trifluoromethyl-phenyl)-acetamide

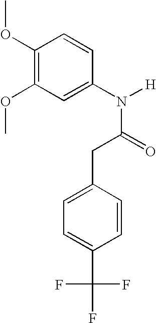

To a 0° C. solution of 7.1 g (34 mmol) 4-(trifluoromethyl)phenylacetic acid in 150 ml dichloromethane were added successively 5 g (32 mmol) 3,4-dimethoxyaniline and 6.6 g (34 mmol) 1-(3-dimethylaminopropyl)-3-ethylcarbodiimide hydrochloride. The mixture was stirred at 0° C. for 30 minutes and then at room temperature for 30 minutes. The solution was washed with a sat. NaHCO₃ solution and with water, dried over Na₂SO₄, filtered and concentrated in vacuo. The crude solid was purified with flash column chromatography on silica eluting with a gradient formed from heptane and ethylacetate to provide 8.7 g (80%) of the title compound as an off-white solid. MS (m/e): 340.4 [M+H]⁺.

### b) Step 2:

### (3,4-Dimethoxy-phenyl)-[2-(4-trifluoromethyl-phenyl)-ethyl]-amine

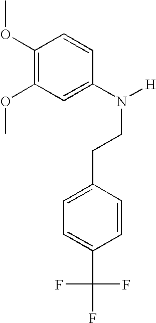

To a solution of 8.7 g (26 mmol) N-(3,4-dimethoxy-phenyl)-2-(4-trifluoromethyl-phenyl)-acetamide in 175 ml THF under argon at room temperature, was added dropwise 51.3 ml (51.3 mmol) of a 1 M borane-tetrahydrofuran solution. The solution was refluxed for 3 hours, cooled to 0° C. and quenched with 120 ml of a 20% NH₄Cl solution. The organic layer was separated and the aqueous layer was extracted once with ethyl acetate. The combined extracts were concentrated in vacuo. The residue was dissolved in 100 ml methanol. The solution was acidified with HCl 5N and stirred at room temperature for 1.5 hours. The solution was basified with a sat. NaHCO₃ solution, and concentrated. The residue was dissolved in ethylacetate, the aqueous phase was extracted 3 times with ethyl acetate. The combined extracts were dried over Na₂SO₄, filtered and concentrated in vacuo. The crude oil was purified with flash column chromatography on silica eluting with a gradient formed from heptane and ethylacetate to provide 7.1 g (85%) of the title compound as a colorless oil. MS (m/e): 326.4 [M+H]⁺.

### c) Step 3:

### 2 (S)—N-(3,4-Dimethoxy-phenyl)-2-hydroxy-2-(2-methoxy-phenyl)-N-[2-(4-trifluoromethyl-phenyl)-ethyl]-acetamide

To a solution of (3,4-dimethoxy-phenyl)-[2-(4-trifluoromethyl-phenyl)-ethyl]-amine (0.414 g, 1.3 mmol) and hydroxy-(2-methoxy-phenyl)-acetic acid (0.232 g, 1.27 mmol) in dry DMF (10 mL) was added HATU (0.484 g, 1.3 mmol) and N-ethyldiisopropylamine (0.22 mL, 1.3 mmol) at rt. After stirring for 15 h, the reaction mixture concentrated, redissolved in EtOAc (15 ml) and washed with aqueous sodium carbonate (half-saturated, 3×10 mL) and H₂O (3×10 mL). The aqueous layers were washed with EtOAc (5 mL). The combined organic layers were dried over sodium sulfate. Concentration and purification by chromatography (SiO₂, heptane:ethyl acetate=2:1) afforded a racemate which was purified by chiral HPLC to give the title compound (0.042 g, 7%) as a colourless oil. MS m/e: 490.3 [M+H]⁺.

### EXAMPLE 38

### 2-Amino-2-(2-methoxy-phenyl)-N-(3-methoxy-phenyl)-N-[2-(4-trifluoromethyl-phenyl)-ethyl]-acetamide

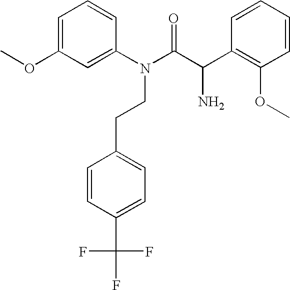

### a) Step 1:

### N-(3-Methoxy-phenyl)-2-(4-trifluoromethyl-phenyl)-acetamide

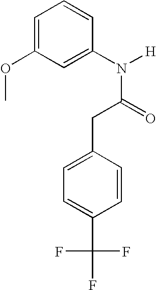

To a 0° C. solution of 1.82 g (8.93 mmol) 4-(trifluoromethyl)phenylacetic acid in 40 ml dichloromethane were added successively 1.0 g (8.12 mmol) 3-methoxyaniline and 1.71 g (8.93 mmol) 1-(3-dimethylaminopropyl)-3-ethylcarbodiimide hydrochloride. The mixture was stirred at 0° C. for 30 minutes and then at room temperature for 30 minutes. The solution was washed with a sat. NaHCO₃ solution and with water, dried over Na₂SO₄, filtered and concentrated in vacuo to provide 2.6 g (>100%) of the title compound as an off-white solid. MS (m/e): 310.1 [M+H]⁺ which was used directly for the next step without further purification.

### b) Step 2:

### (3-Methoxy-phenyl)-[2-(4-trifluoromethyl-phenyl)-ethyl]-amine

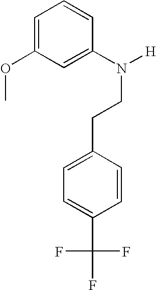

To a solution of 0.928 g (3.0 mmol) N-(3-methoxy-phenyl)-2-(4-trifluoromethyl-phenyl)-acetamide in 15 ml THF under argon at room temperature, was added dropwise 5.9 ml (5.9 mmol) of a 1 M borane-tetrahydrofuran solution. The solution was refluxed for 3 hours, cooled to 0° C. and quenched with 10 ml of a 20% NH₄Cl solution. The organic layer was separated and the aqueous layer was extracted once with ethyl acetate. The combined extracts were concentrated in vacuo. The residue was dissolved in 10 ml methanol. The solution was acidified with HCl 5N and stirred at room temperature for 1.5 hours. The solution was basified with a sat. NaHCO₃ solution, and concentrated. The residue was dissolved in ethylacetate, the aqueous phase was extracted 3 times with ethyl acetate. The combined extracts were dried over Na₂SO₄, filtered and concentrated in vacuo to provide 1.05 g (>100%) of the title compound as a pale yellow oil. MS (m/e): 296.0 [M+H]⁺ which was used in the next step without further purification.

### c) Step 3:

### 2-Amino-2-(2-methoxy-phenyl)-N-(3-methoxy-phenyl)-N-[2-(4-trifluoromethyl-phenyl)-ethyl]-acetamide

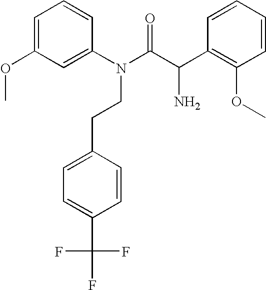

To a 0° C. solution of 0.050 g (0.17 mmol) (3-methoxy-phenyl)-[2-(4-trifluoromethyl-phenyl)-ethyl]-amine and 0.050 g (0.18 mmol) tert-butoxycarbonylamino-(2-methoxy-phenyl)-acetic acid in 1.0 ml dichloromethane under argon, was added 0.18 mmol of 1-(3-dimethylaminopropyl)-3-ethylcarbodiimide hydrochloride. The mixture was stirred at rt for 4 hours. The solution was treated with 1 ml of a 4 M HCl solution in dioxane. The mixture was stirred at room temperature for 18 hours then diluted with CH₂Cl₂ (5 ml) washed once with 5 ml of a sat. NaHCO₃ solution and the solvents were removed in vacuo and DMSO was added and the mixture was purified by prep HPLC to provide the title compound as the free base MS (m/e): 459.2 [M+H]⁺.

### EXAMPLE 39

### 2-Amino-N-(4-chloro-phenyl)-2-(2-methoxy-phenyl)-N-[2-(4-trifluoromethyl-phenyl)-ethyl]-acetamide

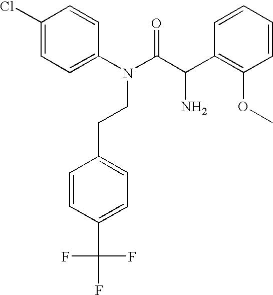

### a) Step 1:

### (4-Chloro-phenyl)-[2-(4-trifluoromethyl-phenyl)-ethyl]-amine

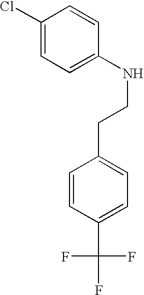

In analogy to the procedure described for the synthesis of example 38 (steps 1 and 2), the title compound was prepared from 4-(trifluoromethyl)phenylacetic acid and 4-chloro-aniline. MS (m/e): 300.0 [M+H]⁺.

### b) Step 2:

### 2-Amino-N-(4-chloro-phenyl)-2-(2-methoxy-phenyl)-N-[2-(4-trifluoromethyl-phenyl)-ethyl]-acetamide

In analogy to the procedure described for the synthesis of example 38 (step 3), the title compound was prepared from (4-chloro-phenyl)-[2-(4-trifluoromethyl-phenyl)-ethyl]-amine and tert-butoxycarbonylamino-(2-methoxy-phenyl)-acetic acid. MS (m/e): 463.1 [M+H]⁺.

### EXAMPLE 40

### 2-Amino-2-(2-methoxy-phenyl)-N-[2-(4-trifluoromethyl-phenyl)-ethyl]-N-(2,4,6-trimethyl-phenyl)-acetamide

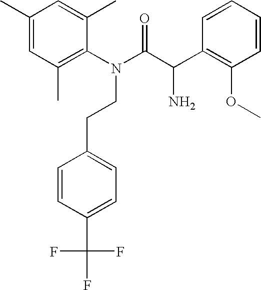

### a) Step 1:

### [2-(4-Trifluoromethyl-phenyl)-ethyl]-(2,4,6-trimethyl-phenyl)-amine

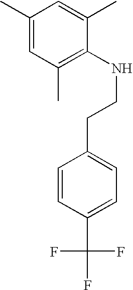

In analogy to the procedure described for the synthesis of example 38 (steps 1 and 2), the title compound was prepared from 4-(trifluoromethyl)phenylacetic acid and 2,4,6-trichloro-aniline. MS (m/e): 308.1 [M+H]⁺.

### b) Step 2:

### 2-Amino-2-(2-methoxy-phenyl)-N-[2-(4-trifluoromethyl-phenyl)-ethyl]-N-(2,4,6-trimethyl-phenyl)-acetamide

In analogy to the procedure described for the synthesis of example 38 (step 3), the title compound was prepared from [2-(4-trifluoromethyl-phenyl)-ethyl]-(2,4,6-trimethyl-phenyl)-amine and tert-butoxycarbonylamino-(2-methoxy-phenyl)-acetic acid. MS (m/e): 471.2 [M+H]⁺.

### EXAMPLE 41

### (2-Amino-N-benzo[1,3]dioxol-5-yl-2-(2-methoxy-phenyl)-N-[2-(4-trifluoromethyl-phenyl)-ethyl]-acetamide

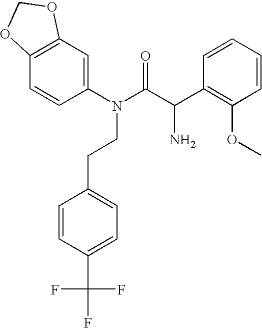

### a) Step 1:

### Benzo[1,3]dioxol-5-yl-[2-(4-trifluoromethyl-phenyl)-ethyl]-amine

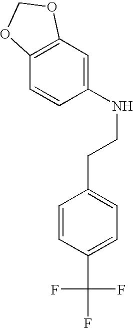

In analogy to the procedure described for the synthesis of example 38 (steps 1 and 2), the title compound was prepared from 4-(trifluoromethyl)phenylacetic acid and benzo[1,3]dioxol-5-ylamine. MS (m/e): 310.0 [M+H]⁺.

### b) Step 2:

### (2-Amino-N-benzo[1,3]dioxol-5-yl-2-(2-methoxy-phenyl)-N-[2-(4-trifluoromethyl-phenyl)-ethyl]-acetamide

In analogy to the procedure described for the synthesis of example 38 (step 3), the title compound was prepared from benzo[1,3]dioxol-5-yl-[2-(4-trifluoromethyl-phenyl)-ethyl]-amine and tert-butoxycarbonylamino-(2-methoxy-phenyl)-acetic acid. MS (m/e): 473.2 [M+H]⁺.

### EXAMPLE 42

### (2-Amino-N-(3-fluoro-4-methoxy-phenyl)-2-(2-methoxy-phenyl)-N-[2-(4-trifluoromethyl-phenyl)-ethyl]-acetamide

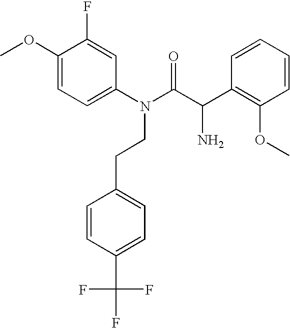

### a) Step 1:

### (3-Fluoro-4-methoxy-phenyl)-[2-(4-trifluoromethyl-phenyl)-ethyl]-amine

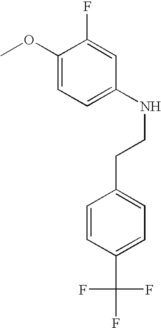

In analogy to the procedure described for the synthesis of example 38 (steps 1 and 2), the title compound was prepared from 4-(trifluoromethyl)phenylacetic acid and 3-fluoro-4-methoxy-phenylamine. MS (m/e): 314.0 [M+H]⁺.

### b) Step 2:

### 2-Amino-N-(3-fluoro-4-methoxy-phenyl)-2-(2-methoxy-phenyl)-N-[2-(4-trifluoromethyl-phenyl)-ethyl]-acetamide

In analogy to the procedure described for the synthesis of example 38 (step 3), the title compound was prepared from (3-fluoro-4-methoxy-phenyl)-[2-(4-trifluoromethyl-phenyl)-ethyl]-amine and tert-butoxycarbonylamino-(2-methoxy-phenyl)-acetic acid. MS (m/e): 477.2 [M+H]⁺.

### EXAMPLE 43

### 2-Amino-N-(3-chloro-4-methoxy-phenyl)-2-(2-methoxy-phenyl)-N-[2-(4-trifluoromethyl-phenyl)-ethyl]-acetamide

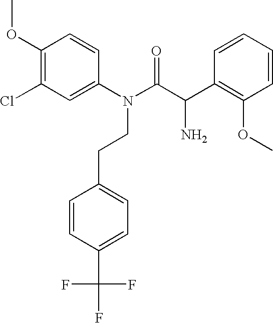

### a) Step 1:

### (3-Chloro-4-methoxy-phenyl)-[2-(4-trifluoromethyl-phenyl)-ethyl]-amine

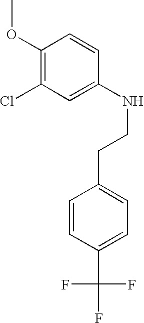

In analogy to the procedure described for the synthesis of example 38 (steps 1 and 2), the title compound was prepared from 4-(trifluoromethyl)phenylacetic acid and 3-chloro-4-methoxy-phenylamine. MS (m/e): 330.0 [M+H]⁺.

### b) Step 2:

### 2-Amino-N-(3-chloro-4-methoxy-phenyl)-2-(2-methoxy-phenyl)-N-[2-(4-trifluoromethyl-phenyl)-ethyl]-acetamide

In analogy to the procedure described for the synthesis of example 38 (step 3), the title compound was prepared from (3-chloro-4-methoxy-phenyl)-[2-(4-trifluoromethyl-phenyl)-ethyl]-amine and tert-butoxycarbonylamino-(2-methoxy-phenyl)-acetic acid. MS (m/e): 493.2 [M+H]⁺.

### EXAMPLE 44

### 2-Amino-2-(2-methoxy-phenyl)-N-(2,2,3,3-tetrafluoro-2,3-dihydro-benzo[1,4]dioxin-6-yl)-N-[2-(4-trifluoromethyl-phenyl)-ethyl]-acetamide

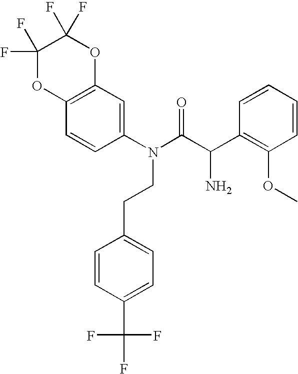

### a) Step 1:

### (2,2,3,3-Tetrafluoro-2,3-dihydro-benzo[1,4]dioxin-6-yl)-[2-(4-trifluoromethyl-phenyl)-ethyl]-amine

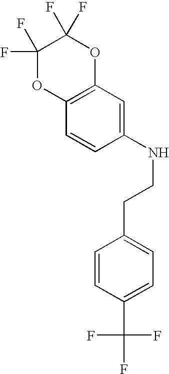

In analogy to the procedure described for the synthesis of example 38 (steps 1 and 2), the title compound was prepared from 4-(trifluoromethyl)phenylacetic acid and 2,2,3,3-tetrafluoro-2,3-dihydro-benzo[1,4]dioxin-6-ylamine. MS (m/e): 395.1 [M+H]⁺.

### b) Step 2:

### 2-Amino-2-(2-methoxy-phenyl)-N-(2,2,3,3-tetrafluoro-2,3-dihydro-benzo[1,4]dioxin-6-yl)-N-[2-(4-trifluoromethyl-phenyl)-ethyl]-acetamide

In analogy to the procedure described for the synthesis of example 38 (step 3), the title compound was prepared from (2,2,3,3-tetrafluoro-2,3-dihydro-benzo[1,4]dioxin-6-yl)-[2-(4-trifluoromethyl-phenyl)-ethyl]-amine and tert-butoxycarbonylamino-(2-methoxy-phenyl)-acetic acid. MS (m/e): 558.3 [M+H]⁺.

### EXAMPLE 45

### 2-Amino-N-(3,4-dimethoxy-phenyl)-2-phenyl-N-[2-(4-trifluoromethyl-phenyl)-ethyl]-acetamide

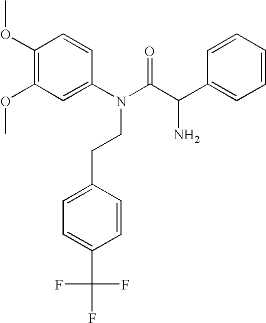

In analogy to the procedure described for the synthesis of example 38 (step 3), the title compound was prepared from (3,4-dimethoxy-phenyl)-[2-(4-trifluoromethyl-phenyl)-ethyl]-amine (prepared as for example 37, step 2) and tert-butoxycarbonylamino-(2-methoxy-phenyl)-acetic acid. MS (m/e): 459.2 [M+H]⁺.

### EXAMPLE 46

### 2-(2-Methoxy-phenyl)-N-(3-methoxy-phenyl)-N-[2-(4-trifluoromethyl-phenyl)-ethyl]-acetamide

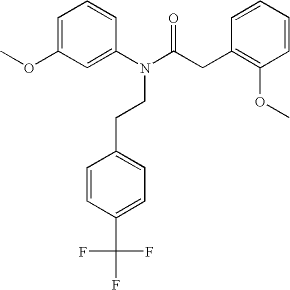

In analogy to the procedure described for the synthesis of example 36 (step 2), the title compound was prepared from (3-methoxy-phenyl)-[2-(4-trifluoromethyl-phenyl)-ethyl]-amine (prepared as for example 38, step 2) and 2-methoxyphenylacetic acid. MS (m/e): 444.3 [M+H]⁺.

### EXAMPLE 47

### 2N-Benzo[1,3]dioxol-5-yl-2-(2-methoxy-phenyl)-N-[2-(4-trifluoromethyl-phenyl)-ethyl]-acetamide

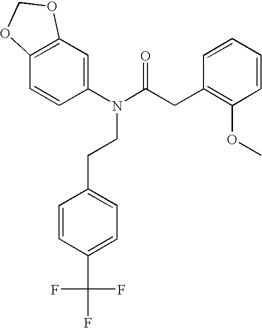

In analogy to the procedure described for the synthesis of example 36 (step 2), the title compound was prepared from benzo[1,3]dioxol-5-yl-[2-(4-trifluoromethyl-phenyl)-ethyl]-amine (prepared as for example 41, step 1) and 2-methoxyphenylacetic acid. MS (m/e): 458.3 [M+H]⁺.

### EXAMPLE 48

### N-(3-Fluoro-4-methoxy-phenyl)-2-(2-methoxy-phenyl)-N-[2-(4-trifluoromethyl-phenyl)-ethyl]-acetamide

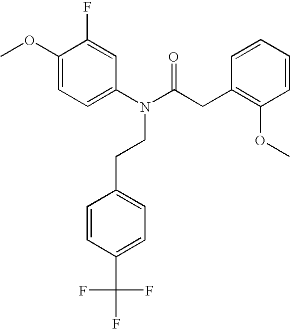

In analogy to the procedure described for the synthesis of example 36 (step 2), the title compound was prepared from (3-fluoro-4-methoxy-phenyl)-[2-(4-trifluoromethyl-phenyl)-ethyl]-amine (prepared as for example 42, step 1) and 2-methoxyphenylacetic acid. MS (m/e): 462.2 [M+H]⁺.

### EXAMPLE 49

### N-(3-Chloro-4-methoxy-phenyl)-2-(2-methoxy-phenyl)-N-[2-(4-trifluoromethyl-phenyl)-ethyl]-acetamide

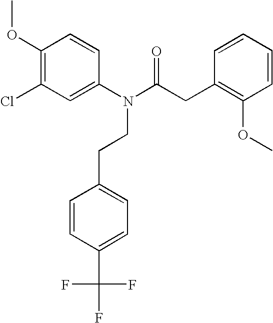

In analogy to the procedure described for the synthesis of example 36 (step 2), the title compound was prepared from (3-chloro-4-methoxy-phenyl)-[2-(4-trifluoromethyl-phenyl)-ethyl]-amine (prepared as for example 43, step 1) and 2-methoxyphenylacetic acid. MS (m/e): 478.2 [M+H]⁺.

### EXAMPLE 50

### N-(3,4-Diethoxy-phenyl)-2-(2-methoxy-phenyl)-N-[2-(4-trifluoromethyl-phenyl)-ethyl]-acetamide

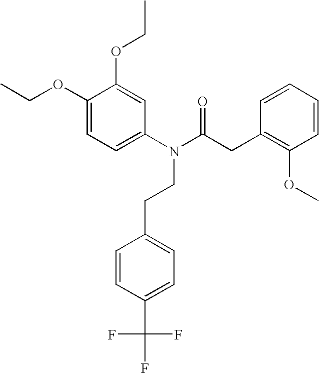

### a) Step 1:

### (3,4-Diethoxy-phenyl)-[2-(4-trifluoromethyl-phenyl)-ethyl]-amine

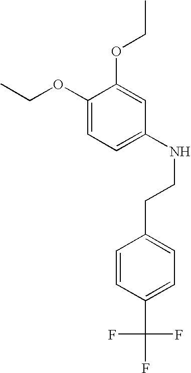

In analogy to the procedure described for the synthesis of example 38 (steps 1 and 2), the title compound was prepared from 4-(trifluoromethyl)phenylacetic acid and 3,4-diethoxy-phenylamine. This material was used directly for the next step.

### b) Step 2:

### N-(3,4-Diethoxy-phenyl)-2-(2-methoxy-phenyl)-N-[2-(4-trifluoromethyl-phenyl)-ethyl]-acetamide

In analogy to the procedure described for the synthesis of example 36 (step 2), the title compound was prepared from (3,4-diethoxy-phenyl)-[2-(4-trifluoromethyl-phenyl)-ethyl]-amine and 2-methoxyphenylacetic acid. MS (m/e): 502.2 [M+H]⁺.

### EXAMPLE 51

### N-Benzothiazol-6-yl-2-(2-methoxy-phenyl)-N-[2-(4-trifluoromethyl-phenyl)-ethyl]-acetamide

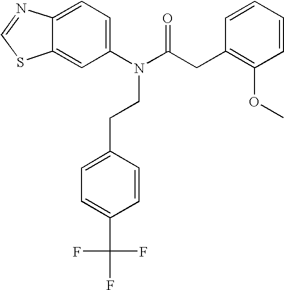

### a) Step 1:

### Benzothiazol-6-yl-[2-(4-trifluoromethyl-phenyl)-ethyl]-amine

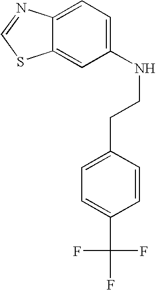

In analogy to the procedure described for the synthesis of example 38 (steps 1 and 2), the title compound was prepared from 4-(trifluoromethyl)phenylacetic acid and benzothiazol-6-ylamine. This material was used directly for the next step.

### b) Step 2:

### N-Benzothiazol-6-yl-2-(2-methoxy-phenyl)-N-[2-(4-trifluoromethyl-phenyl)-ethyl]-acetamide

In analogy to the procedure described for the synthesis of example 36 (step 2), the title compound was prepared from benzothiazol-6-yl-[2-(4-trifluoromethyl-phenyl)-ethyl]-amine and 2-methoxyphenylacetic acid. MS (m/e): 471.0 [M+H]⁺.

### EXAMPLE 52

### N-(4-Chloro-3-methoxy-phenyl)-2-(2-methoxy-phenyl)-N-[2-(4-trifluoromethyl-thenyl)-ethyl]-acetamide

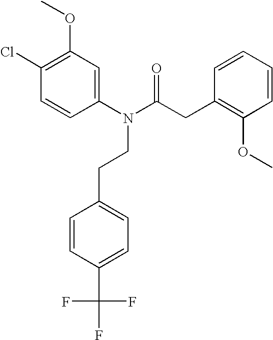

### a) Step 1:

### (4-Chloro-3-methoxy-phenyl)-[2-(4-trifluoromethyl-phenyl)-ethyl]-amine

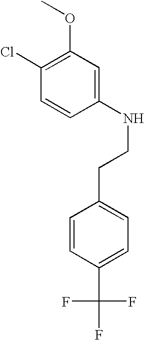

In analogy to the procedure described for the synthesis of example 38 (steps 1 and 2), the title compound was prepared from 4-(trifluoromethyl)phenylacetic acid and 4-chloro-3-methoxy-phenylamine. MS (m/e): 330.0 [M+H]⁺.

### b) Step 2:

### N-(3,4-Diethoxy-phenyl)-2-(2-methoxy-phenyl)-N-[2-(4-trifluoromethyl-phenyl)-ethyl]-acetamide

In analogy to the procedure described for the synthesis of example 36 (step 2), the title compound was prepared from (4-chloro-3-methoxy-phenyl)-[2-(4-trifluoromethyl-phenyl)-ethyl]-amine and 2-methoxyphenylacetic acid. MS (m/e): 478.2 [M+H]⁺.

### EXAMPLE 53

### N-(3,4-Dimethoxy-phenyl)-2-methylamino-2-phenyl-N-[2-(4-trifluoromethyl-phenyl)-ethyl]-acetamide

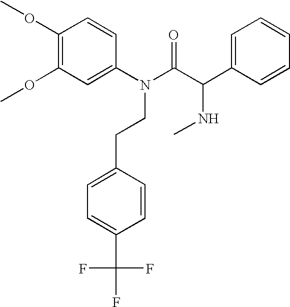

### a) Step 1:

### ({(3,4-Dimethoxy-phenyl)-[2-(4-trifluoromethyl-phenyl)-ethyl]-carbamoyl}-phenyl-methyl)-carbamic acid tert-butyl ester

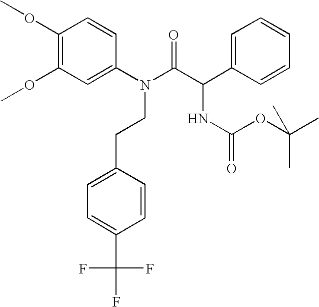

To a 0° C. solution of 1.50 g (0.46 mmol) (3,4-dimethoxy-phenyl)-[2-(4-trifluoromethyl-phenyl)-ethyl]-amine and (0.46 mmol) tert-butoxycarbonylamino-(2-methoxy-phenyl)-acetic acid in 5.0 ml dichloroethane under argon, was added 4.83 mmol of HATU and 4.83 mmol Et₃N. The mixture was stirred at 80° for 24 hours, cooled to rt then washed once with 5 ml H₂O and the organic layer concentrated and purified by prep HPLC to provide the title compound MS (m/e): 559.3 [M+H]⁺.

### b) Step 2:

### ({(3,4-Dimethoxy-phenyl)-[2-(4-trifluoromethyl-phenyl)-ethyl]-carbamoyl}-phenyl-methyl)-methyl-carbamic acid tert-butyl ester

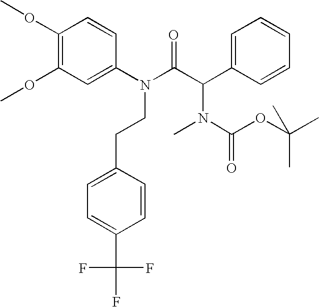

To a 0° C. solution of 0.50 g (0.90 mmol) 2-amino-2-(2-methoxy-phenyl)-N-(3-methoxy-phenyl)-N-[2-(4-trifluoromethyl-phenyl)-ethyl]-acetamide in 5.0 ml DMF was added 1.1 eq Me I and 1.1 eq NaH and the mixture stirred at rt for 1 h. After cooling to 0°, the mixture was treated with 5 ml H₂O, then extracted with (3×5 ml) EtOAc, the organic layer concentrated and purified by prep HPLC to provide the title compound MS (m/e): 573.4 [M+H]⁺.

### c) Step 3:

### N-(3,4-Dimethoxy-phenyl)-2-methylamino-2-phenyl-N-[2-(4-trifluoromethyl-phenyl)-ethyl]-acetamide

To a solution of 100 mg (0.18 mmol) ({(3,4-dimethoxy-phenyl)-[2-(4-trifluoromethyl-phenyl)-ethyl]-carbamoyl}-phenyl-methyl)-methyl-carbamic acid tert-butyl ester in 1 mL CH₂Cl₂ was added 10 eq of TFA. After 1 h, the mixture was diluted with CH₂Cl₂ (5 ml) washed once with 5 ml of a sat. NaHCO₃ solution and the solvents were removed in vacuo and DMSO was added and the mixture was purified by prep HPLC to provide the title compound as the free base MS (m/e): 473.3 [M+H]⁺.

### EXAMPLE 54

### 2-(2-Methoxy-phenyl)-N-p-tolyl-N-[2-(4-trifluoromethyl-phenyl)-ethyl]-acetamide

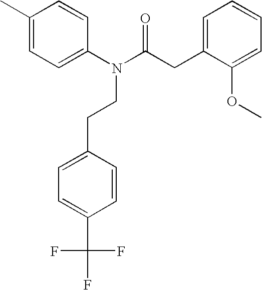

### a) Step 1:

### p-Tolyl-[2-(4-trifluoromethyl-phenyl)-ethyl]-amine

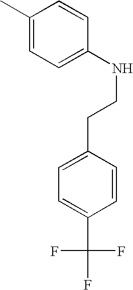

In analogy to the procedure described for the synthesis of example 38 (steps 1 and 2), the title compound was prepared from 4-(trifluoromethyl)phenylacetic acid and p-tolylamine. MS (m/e): 280.1 [M+H]⁺.

### b) Step 2:

### 2-(2-Methoxy-phenyl)-N-p-tolyl-N-[2-(4-trifluoromethyl-phenyl)-ethyl]-acetamide

In analogy to the procedure described for the synthesis of example 36 (step 2), the title compound was prepared from p-tolyl-[2-(4-trifluoromethyl-phenyl)-ethyl]-amine and 2-methoxyphenylacetic acid. MS (m/e): 428.3 [M+H]⁺.

### EXAMPLE 55

### 2-Amino-N-(4-chloro-phenyl)-2-phenyl-N-[2-(4-trifluoromethyl-phenyl)-ethyl]-acetamide

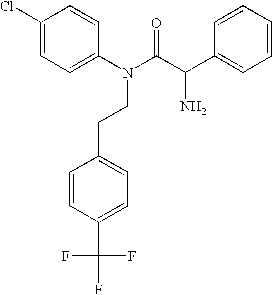

To a solution of (0.18 mmol) tert-butoxycarbonylamino-phenyl-acetic acid, 2-chloro-4,6-dimethoxy-1,3,5-triazine (1.05 eq), N-methylmorpholine (1.5 eq) in (2.0 ml) EtOAc at rt was added (0.17 mmol) (4-chloro-phenyl)-[2-(4-trifluoromethyl-phenyl)-ethyl]-amine (prepared as for example 39, step 1) and the mixture stirred for 16 h. Filtration of the white ppt, concentration and re-dissolution in (1 ml) CH₂Cl₂ was followed by treatment with (10 eq) TFA. The mixture was stirred at room temperature for 18 hours then diluted with CH₂Cl₂ (5 ml) washed once with 5 ml of a sat. NaHCO₃ solution and the solvents were removed in vacuo and DMSO was added and the mixture was purified by prep HPLC to provide the title compound as the free base MS (m/e): 433.2 [M+H]⁺.

### EXAMPLE 56

### 2-Amino-N-(3-chloro-phenyl)-2-phenyl-N-[2-(4-trifluoromethyl-phenyl)-ethyl]acetamide

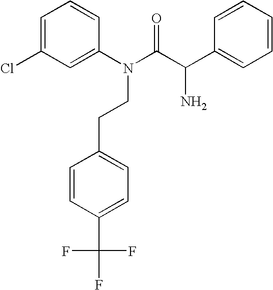

### a) Step 1:

### (3-Chloro-4-methoxy-phenyl)-[2-(4-trifluoromethyl-phenyl)-ethyl]-amine

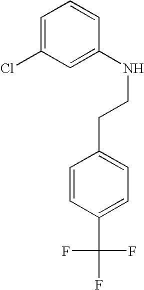

In analogy to the procedure described for the synthesis of example 38 (steps 1 and 2), the title compound was prepared from 4-(trifluoromethyl)phenylacetic acid and 3-chloro-phenylamine. MS (m/e): 300.0 [M+H]⁺.

### b) Step 2:

### 2-Amino-N-(3-chloro-phenyl)-2-phenyl-N-[2-(4-trifluoromethyl-phenyl)-ethyl]acetamide

In analogy to the procedure described for the synthesis of example 55, the title compound was prepared from (3-chloro-4-methoxy-phenyl)-[2-(4-trifluoromethyl-phenyl)-ethyl]-amine and tert-butoxycarbonylamino-phenyl-acetic acid. MS (m/e): 433.2 [M+H]⁺.

### EXAMPLE 57

### 2-Amino-2-phenyl-N-[2-(4-trifluoromethyl-phenyl)-ethyl]-N-(2,4,6-trimethyl-phenyl)-acetamide

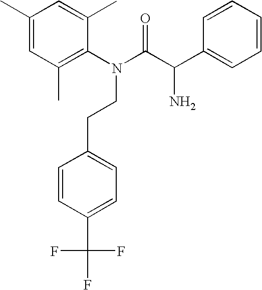

In analogy to the procedure described for the synthesis of example 55, the title compound was prepared from [2-(4-trifluoromethyl-phenyl)-ethyl]-(2,4,6-trimethyl-phenyl)-amine (prepared in example 40, step 1) and tert-butoxycarbonylamino-phenyl-acetic acid. MS (m/e): 441.3 [M+H]⁺.

### EXAMPLE 58

### 2-Amino-N-benzo[1,3]dioxol-5-yl-2-phenyl-N-[2-(4-trifluoromethyl-phenyl)-ethyl]-acetamide

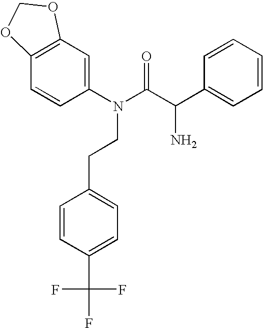

In analogy to the procedure described for the synthesis of example 55, the title compound was prepared from benzo[1,3]dioxol-5-yl-[2-(4-trifluoromethyl-phenyl)-ethyl]-amine (prepared in example 41, step 1) and tert-butoxycarbonylamino-phenyl-acetic acid. MS (m/e): 443.3 [M+H]⁺.

### EXAMPLE 59

### 2-Amino-N-(5-methoxy-2-methyl-phenyl)-2-phenyl-N-[2-(4-trifluoromethyl-phenyl)-ethyl]-acetamide

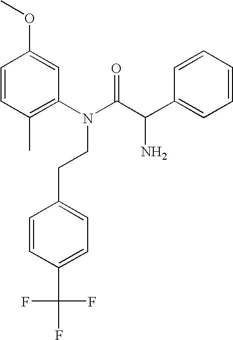

### a) Step 1:

### (5-Methoxy-2-methyl-phenyl)-[2-(4-trifluoromethyl-phenyl)-ethyl]-amine

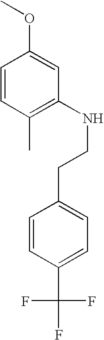

In analogy to the procedure described for the synthesis of example 38 (steps 1 and 2), the title compound was prepared from 4-(trifluoromethyl)phenylacetic acid and 5-methoxy-2-methyl-phenylamine. MS (m/e): 310.0 [M+H]⁺.

### b) Step 2:

### 2-Amino-N-(5-methoxy-2-methyl-phenyl)-2-phenyl-N-[2-(4-trifluoromethyl-phenyl)-ethyl]-acetamide

In analogy to the procedure described for the synthesis of example 55, the title compound was prepared from (5-methoxy-2-methyl-phenyl)-[2-(4-trifluoromethyl-phenyl)-ethyl]-amine and tert-butoxycarbonylamino-phenyl-acetic acid. MS (m/e): 443.3 [M+H]⁺.

### EXAMPLE 60

### 2-Amino-N-(3-methoxy-phenyl)-2-phenyl-N-[2-(4-trifluoromethyl-phenyl)-ethyl]-acetamide

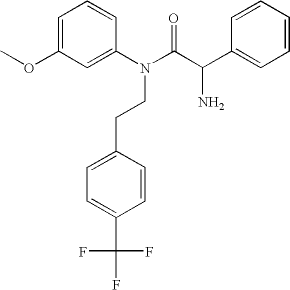

In analogy to the procedure described for the synthesis of example 55, the title compound was prepared from (3-methoxy-phenyl)-[2-(4-trifluoromethyl-phenyl)-ethyl]-amine (prepared in example 38, steps 1 & 2) and tert-butoxycarbonylamino-phenyl-acetic acid. MS (m/e): 429.3 [M+H]⁺.

### EXAMPLE 61

### 2-Amino-N-(3,4-dimethoxy-phenyl)-2-(4-fluoro-phenyl)-N-[2-(4-trifluoromethyl-phenyl)-ethyl]-acetamide

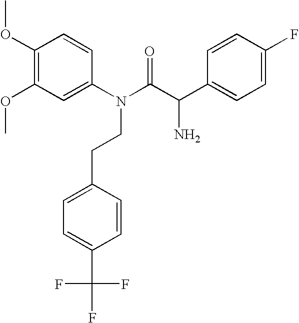

In analogy to the procedure described for the synthesis of example 55 (step 3), the title compound was prepared from (3,4-dimethoxy-phenyl)-[2-(4-trifluoromethyl-phenyl)-ethyl]-amine (prepared in example 37, steps 1 & 2) and amino-(4-fluoro-phenyl)-acetic acid. MS (m/e): 477.3 [M+H]⁺.

### EXAMPLE 62

### 2-Amino-N-(3,4-dimethoxy-phenyl)-2-(4-fluoro-phenyl)-N-[2-(4-trifluoromethyl-phenyl)-ethyl]-acetamide

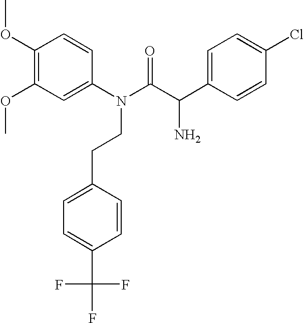

In analogy to the procedure described for the synthesis of example 55, the title compound was prepared from (3,4-dimethoxy-phenyl)-[2-(4-trifluoromethyl-phenyl)-ethyl]-amine (prepared in example 37, steps 1 & 2) and amino-(4-chloro-phenyl)-acetic acid. MS (m/e): 493.3 [M+H]⁺.

### EXAMPLE 63

### 2-Amino-2-(3-chloro-phenyl)-N-(3,4-dimethoxy-phenyl)-N-[2-(4-trifluoromethyl-phenyl)-ethyl]-acetamide

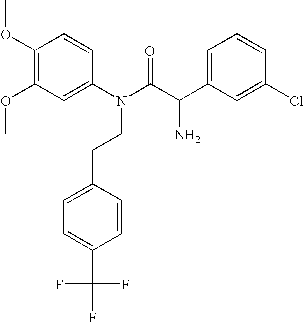

In analogy to the procedure described for the synthesis of example 55, the title compound was prepared from (3,4-dimethoxy-phenyl)-[2-(4-trifluoromethyl-phenyl)-ethyl]-amine (prepared in example 37, steps 1 & 2) and amino-(3-chloro-phenyl)-acetic acid. MS (m/e): 493.3 [M+H]⁺.

### EXAMPLE 64

### 2-Amino-N-(3-fluoro-4-methoxy-phenyl)-2-phenyl-N-[2-(4-trifluoromethyl-phenyl)-ethyl]-acetamide

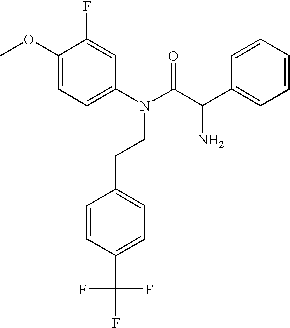

In analogy to the procedure described for the synthesis of example 55, the title compound was prepared from (3-fluoro-4-methoxy-phenyl)-[2-(4-trifluoromethyl-phenyl)-ethyl]-amine (prepared in example 42, step 1) and tert-Butoxycarbonylamino-phenyl-acetic acid. MS (m/e): 447.3 [M+H]⁺.

### EXAMPLE 65

### 2-Amino-N-(4-chloro-3-methoxy-phenyl)-2-phenyl-N-[2-(4-trifluoromethyl-phenyl)-ethyl]-acetamide

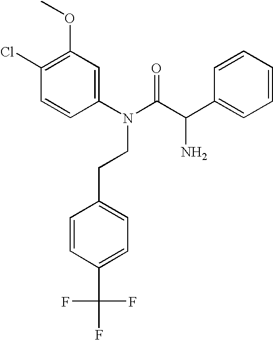

In analogy to the procedure described for the synthesis of example 55, the title compound was prepared from (4-chloro-3-methoxy-phenyl)-[2-(4-trifluoromethyl-phenyl)-ethyl]-amine (prepared in example 52, step 1) and tert-butoxycarbonylamino-phenyl-acetic acid. MS (m/e): 463.2 [M+H]⁺.

### EXAMPLE 66

### 2-Amino-N-(3-chloro-4-methoxy-phenyl)-2-phenyl-N-[2-(4-trifluoromethyl-phenyl)-ethyl]-acetamide

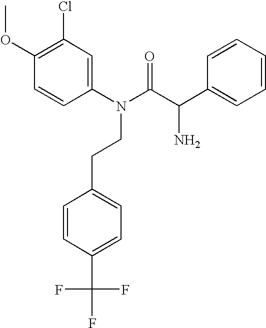

In analogy to the procedure described for the synthesis of example 55, the title compound was prepared from (3-chloro-4-methoxy-phenyl)-[2-(4-trifluoromethyl-phenyl)-ethyl]-amine (prepared in example 43, step 1) and tert-butoxycarbonylamino-phenyl-acetic acid. MS (m/e): 463.2 [M+H]⁺.

### EXAMPLE 67

### 2-Amino-N-(2,2-difluoro-benzo[1,3]dioxol-5-yl)-2-phenyl-N-[2-(4-trifluoromethyl-phenyl)-ethyl]-acetamide

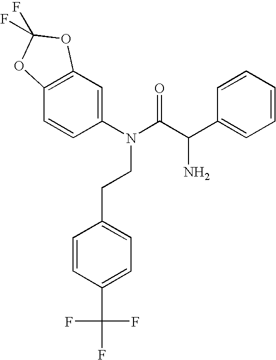

### a) Step 1:

### (2,2-Difluoro-benzo[1,3]dioxol-5-yl)-[2-(4-trifluoromethyl-phenyl)-ethyl]-amine

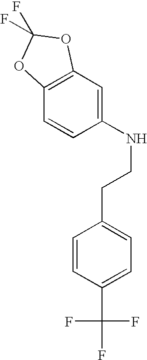

In analogy to the procedure described for the synthesis of example 38 (steps 1 and 2), the title compound was prepared from 4-(trifluoromethyl)phenylacetic acid and 2,2-difluoro-benzo[1,3]dioxol-5-ylamine. MS (m/e): 387.1 [M+H: CH₃CN adduct]⁺.

### b) Step 2:

### 2-Amino-N-(2,2-difluoro-benzo[1,3]dioxol-5-yl)-2-phenyl-N-[2-(4-trifluoromethyl-phenyl)-ethyl]-acetamide

In analogy to the procedure described for the synthesis of example 55, the title compound was prepared from (2,2-difluoro-benzo[1,3]dioxol-5-yl)-[2-(4-trifluoromethyl-phenyl)-ethyl]-amine and tert-butoxycarbonylamino-phenyl-acetic acid. MS (m/e): 479.2 [M+H]⁺.

### EXAMPLE 68

### 2-Amino-N-(3,4-diethoxy-phenyl)-2-phenyl-N-[2-(4-trifluoromethyl-phenyl)-ethyl]-acetamide

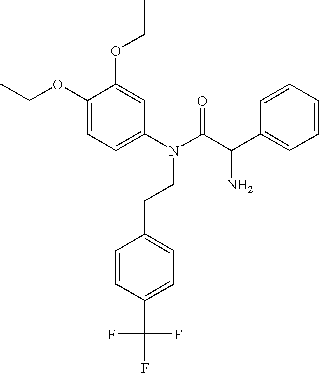

In analogy to the procedure described for the synthesis of example 55, the title compound was prepared from (3,4-diethoxy-phenyl)-[2-(4-trifluoromethyl-phenyl)-ethyl]-amine (prepared in example 50, step 1) and tert-butoxycarbonylamino-phenyl-acetic acid. MS (m/e): 487.3 [M+H]⁺.

### EXAMPLE 69

### 2-Amino-2-phenyl-N-p-tolyl-N-[2-(4-trifluoromethyl-phenyl)-ethyl]-acetamide

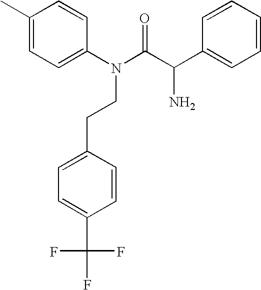

In analogy to the procedure described for the synthesis of example 55, the title compound was prepared from p-tolyl-[2-(4-trifluoromethyl-phenyl)-ethyl]-amine (prepared in example 54, step 1) and tert-butoxycarbonylamino-phenyl-acetic acid. MS (m/e): 413.3 [M+H]⁺.

### EXAMPLE 70

### 2-Amino-2-phenyl-N-m-tolyl-N-[2-(4-trifluoromethyl-phenyl)-ethyl]-acetamide

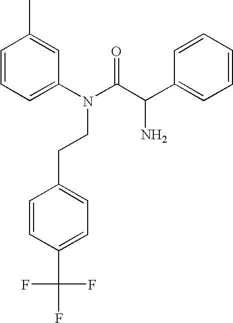

In analogy to the procedure described for the synthesis of example 55, the title compound was prepared from m-tolyl-[2-(4-trifluoromethyl-phenyl)-ethyl]-amine (prepared in example 36, step 1) and tert-butoxycarbonylamino-phenyl-acetic acid. MS (m/e): 413.3 [M+H]⁺.

### EXAMPLE 71

### 2-Amino-2-phenyl-N-(4-trifluoromethoxy-phenyl)-N-[2-(4-trifluoromethyl-phenyl)-ethyl]-acetamide

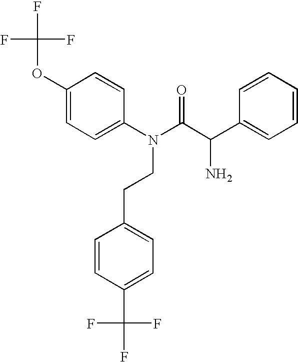

### a) Step 1:

### (4-Trifluoromethoxy-phenyl)-[2-(4-trifluoromethyl-phenyl)-ethyl]-amine

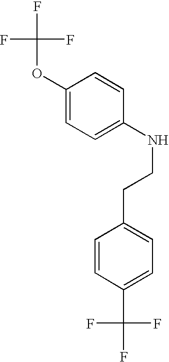

In analogy to the procedure described for the synthesis of example 38 (steps 1 and 2), the title compound was prepared from 4-(trifluoromethyl)phenylacetic acid and 4-trifluoromethoxy-phenylamine. MS (m/e): 391.1 [M+H]⁺.

### b) Step 2:

### 2-Amino-2-phenyl-N-(4-trifluoromethoxy-phenyl)-N-[2-(4-trifluoromethyl-phenyl)-ethyl]-acetamide

In analogy to the procedure described for the synthesis of example 55, the title compound was prepared from (4-Trifluoromethoxy-phenyl)-[2-(4-trifluoromethyl-phenyl)-ethyl]-amine and tert-Butoxycarbonylamino-phenyl-acetic acid. MS (m/e): 483.3 [M+H]⁺.

### EXAMPLE 72

### (S)—N-(3,4-Dimethoxy-phenyl)-2-(oxetan-3-ylamino)-2-phenyl-N-[2-(4-trifluoromethyl-phenyl)-ethyl]-acetamide

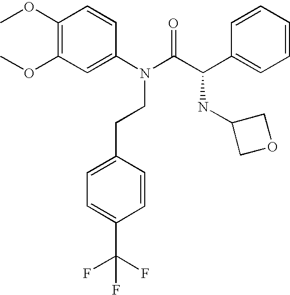

To a solution of 80 mg (0.174 mmol) (S)-2-Amino-N-(3,4-dimethoxy-phenyl)-2-phenyl-N-[2-(4-trifluoromethyl-phenyl)-ethyl]-acetamide (example 173) in 1,2-dichloroethane (0.6 ml) were added 12 ul (0.174 mmol) 3-oxetanone, 55 mg (0.244 mmol) sodium triacetoxyborohydride and finally 10 ul (0.174 mmol) acetic acid. The mixture was stirred at room temperature for 22 hours. The mixture was quenched with a 1N NaOH solution and extracted 3 times with dichloromethane. The combined extracts were dried over Na₂SO₄, filtered and concentrated in vacuo. The crude solid was purified with flash column chromatography on silica eluting with a gradient formed from heptane and ethylacetate to provide 20 mg (22%) of the title compound as alight yellow oil. MS (m/e): 515.4 [M+H]⁺.

### EXAMPLE 73

### 2-Amino-N-(3,4-dimethoxy-phenyl)-N-[2-(4-methanesulfonyl-phenyl)-ethyl]-2-phenyl-acetamide

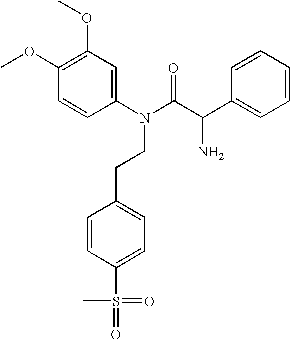

### a) Step 1:

### (3,4-Dimethoxy-phenyl)-[2-(4-methanesulfonyl-phenyl)-ethyl]-amine

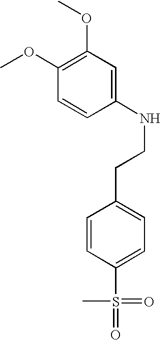

In analogy to the procedure described for the synthesis of example 38 (steps 1 and 2), the title compound was prepared from (4-methanesulfonyl-phenyl)-acetic acid and 3,4-dimethoxy-phenylamine. MS (m/e): 336.0 [M+H]⁺.

### b) Step 2:

### 2-Amino-N-(3,4-dimethoxy-phenyl)-N-[2-(4-methanesulfonyl-phenyl)-ethyl]-2-phenyl-acetamide

In analogy to the procedure described for the synthesis of example 55, the title compound was prepared from (4-trifluoromethoxy-phenyl)-[2-(4-trifluoromethyl-phenyl)-ethyl]-amine and tert-butoxycarbonylamino-phenyl-acetic acid. MS (m/e): 469.3 [M+H]⁺.

### EXAMPLE 74

### 2-Amino-N-(3,4-dimethoxy-phenyl)-N-[2-(3,4-dimethoxy-phenyl)-ethyl]-2-phenyl-acetamide

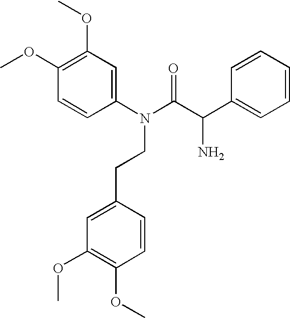

### a) Step 1:

### (3,4-Dimethoxy-phenyl)-[2-(3,4-dimethoxy-phenyl)-ethyl]-amine

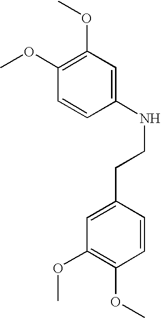

In analogy to the procedure described for the synthesis of example 38 (steps 1 and 2), the title compound was prepared from (3,4-dimethoxy-phenyl)-acetic acid and 3,4-dimethoxy-phenylamine. MS (m/e): 318.0 [M+H]⁺_{.}

### b) Step 2:

### 2-Amino-N-(3,4-dimethoxy-phenyl)-N-[2-(4-methanesulfonyl-phenyl)-ethyl]-2-phenyl-acetamide

In analogy to the procedure described for the synthesis of example 55, the title compound was prepared from (3,4-dimethoxy-phenyl)-[2-(3,4-dimethoxy-phenyl)-ethyl]-amine and tert-butoxycarbonylamino-phenyl-acetic acid. MS (m/e): 451.3 [M+H]⁺.

### EXAMPLE 75

### 2-Amino-N-(4-methoxy-phenyl)-2-phenyl-N-[2-(4-trifluoromethyl-phenyl)-ethyl]-acetamide

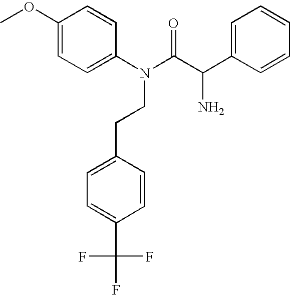

### a) Step 1:

### (4-Methoxy-phenyl)-[2-(4-trifluoromethyl-phenyl)-ethyl]-amine

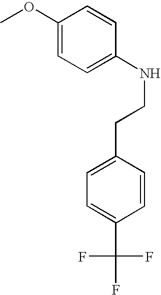

In analogy to the procedure described for the synthesis of example 38 (steps 1 and 2), the title compound was prepared from 4-methoxy-phenylamine and (4-trifluoromethyl-phenyl)-acetic acid. MS (m/e): 296.0 [M+H]⁺.

### b) Step 2:

### 2-Amino-N-(4-methoxy-phenyl)-2-phenyl-N-[2-(4-trifluoromethyl-phenyl)-ethyl]-acetamide

In analogy to the procedure described for the synthesis of example 55, the title compound was prepared from (4-methoxy-phenyl)-[2-(4-trifluoromethyl-phenyl)-ethyl]-amine and tert-butoxycarbonylamino-phenyl-acetic acid. MS (m/e): 429.3 [M+H]⁺.

### EXAMPLE 76

### 2-Amino-N-[2-(3,4-dimethoxy-phenyl)-ethyl]-N-(4-methoxy-phenyl)-2-phenyl-acetamide

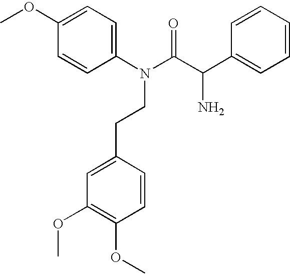

### a) Step 1:

### (3,4-Dimethoxy-phenyl)-[2-(3,4-dimethoxy-phenyl)-ethyl]-amine

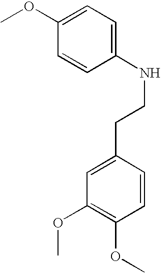

In analogy to the procedure described for the synthesis of example 38 (steps 1 and 2), the title compound was prepared from (3,4-dimethoxy-phenyl)-acetic acid and 4-methoxy-phenylamine. MS (m/e): 288.1 [M+H]⁺.

### b) Step 2:

### 2-Amino-N-[2-(3,4-dimethoxy-phenyl)-ethyl]-N-(4-methoxy-phenyl)-2-phenyl-acetamide

In analogy to the procedure described for the synthesis of example 55, the title compound was prepared from (3,4-dimethoxy-phenyl)-[2-(3,4-dimethoxy-phenyl)-ethyl]-amine and tert-butoxycarbonylamino-phenyl-acetic acid. MS (m/e): 421.3 [M+H]⁺.

### EXAMPLE 77

### 2-Amino-N-[2-(4-methanesulfonyl-phenyl)-ethyl]-2-phenyl-N-(4-trifluoromethoxy-phenyl)-acetamide

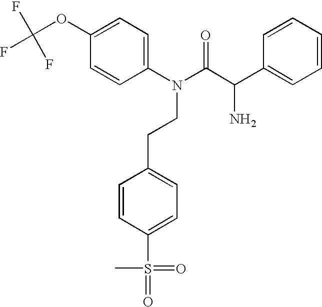

### a) Step 1:

### [2-(4-Methanesulfonyl-phenyl)-ethyl]-(4-trifluoromethoxy-phenyl)-amine

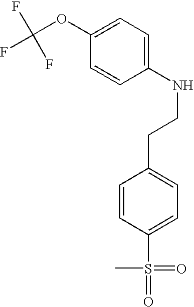

In analogy to the procedure described for the synthesis of example 38 (steps 1 and 2), the title compound was prepared from (4-methanesulfonyl-phenyl)-acetic acid and 4-trifluoromethoxy-phenylamine. MS (m/e): 401.1 [M+H+CH₃CN]⁺.

### b) Step 2:

### 2-Amino-N-[2-(4-methanesulfonyl-phenyl)-ethyl]-2-phenyl-N-(4-trifluoromethoxy-phenyl)-acetamide

In analogy to the procedure described for the synthesis of example 55, the title compound was prepared from [2-(4-methanesulfonyl-phenyl)-ethyl]-(4-trifluoromethoxy-phenyl)-amine and tert-butoxycarbonylamino-phenyl-acetic acid. MS (m/e): 493.2 [M+H]⁺.

### EXAMPLE 78

### 2-Amino-N-[2-(3,4-dimethoxy-phenyl)-ethyl]-2-phenyl-N-(4-trifluoromethoxy-phenyl)-acetamide

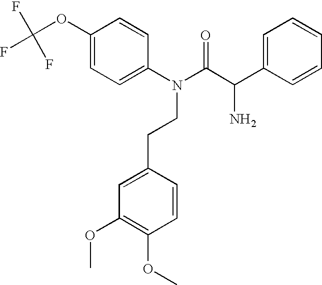

### a) Step 1:

### [2-(3,4-Dimethoxy-phenyl)-ethyl]-(4-trifluoromethoxy-phenyl)-amine

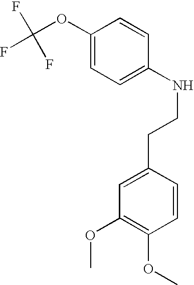

In analogy to the procedure described for the synthesis of example 38 (steps 1 and 2), the title compound was prepared from 4-trifluoromethoxy-phenylamine and 4-trifluoromethoxy-phenylamine. MS (m/e): 342.1 [M+H]⁺.

### b) Step 2:

### 2-Amino-N-[2-(3,4-dimethoxy-phenyl)-ethyl]-2-phenyl-N-(4-trifluoromethoxy-phenyl)-acetamide

In analogy to the procedure described for the synthesis of example 55, the title compound was prepared from [2-(3,4-dimethoxy-phenyl)-ethyl]-(4-trifluoromethoxy-phenyl)-amine and tert-butoxycarbonylamino-phenyl-acetic acid. MS (m/e): 475.2 [M+H]⁺.

### EXAMPLE 79

### 2-Amino-N-(2,4-difluoro-phenyl)-2-phenyl-N-[2-(4-trifluoromethyl-phenyl)-ethyl]-acetamide

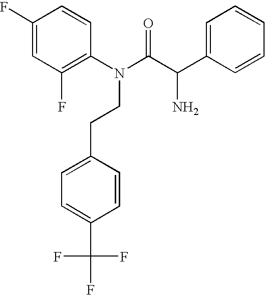

### a) Step 1:

### (2,4-Difluoro-phenyl)-[2-(4-trifluoromethyl-phenyl)-ethyl]-amine

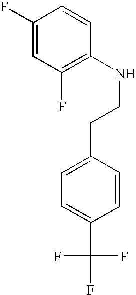

In analogy to the procedure described for the synthesis of example 38 (steps 1 and 2), the title compound was prepared from 2,4-difluoro-phenylamine and (4-trifluoromethyl-phenyl)-acetic acid. MS (m/e): 302.0 [M+H]⁺.

### b) Step 2:

### 2-Amino-N-(2,4-difluoro-phenyl)-2-phenyl-N-[2-(4-trifluoromethyl-phenyl)-ethyl]-acetamide

In analogy to the procedure described for the synthesis of example 55, the title compound was prepared from (2,4-difluoro-phenyl)-[2-(4-trifluoromethyl-phenyl)-ethyl]-amine and tert-butoxycarbonylamino-phenyl-acetic acid. MS (m/e): 435.3 [M+H]⁺.

### EXAMPLE 80

### 2-Amino-N-(2,4-difluoro-phenyl)-N-[2-(3,4-dimethoxy-phenyl)-ethyl]-2-phenyl-acetamide

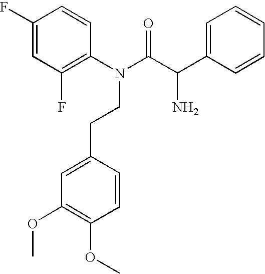

### a) Step 1:

### (2,4-Difluoro-phenyl)-[2-(3,4-dimethoxy-phenyl)-ethyl]-amine

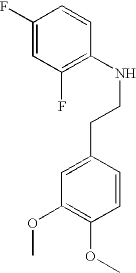

In analogy to the procedure described for the synthesis of example 38 (steps 1 and 2), the title compound was prepared from 2,4-difluoro-phenylamine and (3,4-dimethoxy-phenyl)-acetic acid. MS (m/e): 294.0 [M+H]⁺.

### b) Step 2:

### 2-Amino-N-(2,4-difluoro-phenyl)-N-[2-(3,4-dimethoxy-phenyl)-ethyl]-2-phenyl-acetamide

In analogy to the procedure described for the synthesis of example 55, the title compound was prepared from (2,4-difluoro-phenyl)-[2-(3,4-dimethoxy-phenyl)-ethyl]-amine and tert-butoxycarbonylamino-phenyl-acetic acid. MS (m/e): 427.3 [M+H]⁺.

### EXAMPLE 81

### 2-Amino-2-phenyl-N-(3-trifluoromethyl-phenyl)-N-[2-(4-trifluoromethyl-phenyl)-ethyl]-acetamide

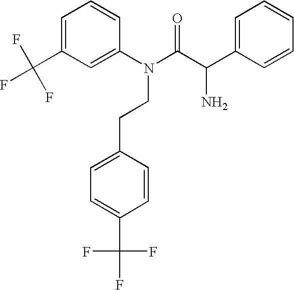

### a) Step 1:

### (3-Trifluoromethyl-phenyl)-[2-(4-trifluoromethyl-phenyl)-ethyl]-amine

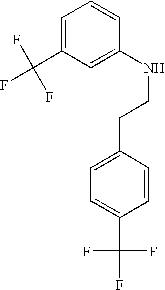

In analogy to the procedure described for the synthesis of example 38 (steps 1 and 2), the title compound was prepared from 3-trifluoromethyl-phenylamine and (4-trifluoromethyl-phenyl)-acetic acid. This material was used directly for the next step.

### b) Step 2:

### 2-Amino-2-phenyl-N-(3-trifluoromethyl-phenyl)-N-[2-(4-trifluoromethyl-phenyl)-ethyl]-acetamide

In analogy to the procedure described for the synthesis of example 55, the title compound was prepared from (3-trifluoromethyl-phenyl)-[2-(4-trifluoromethyl-phenyl)-ethyl]-amine and tert-butoxycarbonylamino-phenyl-acetic acid. MS (m/e): 467.3 [M+H]⁺.

### EXAMPLE 82

### 2-Amino-N-(4-chloro-3-methyl-phenyl)-2-phenyl-N-[2-(4-trifluoromethyl-phenyl)-ethyl]-acetamide

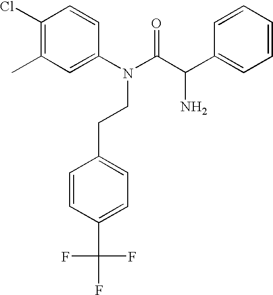

### a) Step 1:

### (4-Chloro-3-methyl-phenyl)-[2-(4-trifluoromethyl-phenyl)-ethyl]-amine

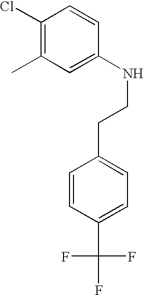

In analogy to the procedure described for the synthesis of example 38 (steps 1 and 2), the title compound was prepared from 4-chloro-3-methyl-phenylamine and (4-trifluoromethyl-phenyl)-acetic acid MS (m/e): 314.3 [M+H]⁺.

### b) Step 2:

### 2-Amino-N-(4-chloro-3-methyl-phenyl)-2-phenyl-N-[2-(4-trifluoromethyl-phenyl)-ethyl]-acetamide

In analogy to the procedure described for the synthesis of example 55, the title compound was prepared from (4-Chloro-3-methyl-phenyl)-[2-(4-trifluoromethyl-phenyl)-ethyl]-amine and tert-butoxycarbonylamino-phenyl-acetic acid. MS (m/e): 447.3 [M+H]⁺.

### EXAMPLE 83

### 2-Amino-N-(4-fluoro-3-methyl-phenyl)-2-phenyl-N-[2-(4-trifluoromethyl-phenyl)-ethyl]-acetamide

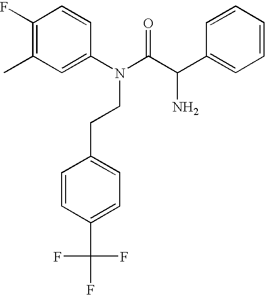

### a) Step 1:

### (4-Fluoro-3-methyl-phenyl)-[2-(4-trifluoromethyl-phenyl)-ethyl]-amine

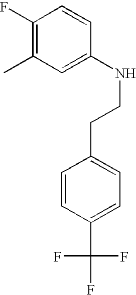

In analogy to the procedure described for the synthesis of example 38 (steps 1 and 2), the title compound was prepared from 4-fluoro-3-methyl-phenylamine and (4-trifluoromethyl-phenyl)-acetic acid MS (m/e): 298.4 [M+H]⁺.

### b) Step 2:

### 2-Amino-N-(4-fluoro-3-methyl-phenyl)-2-phenyl-N-[2-(4-trifluoromethyl-phenyl)-ethyl]-acetamide

In analogy to the procedure described for the synthesis of example 55, the title compound was prepared from (4-fluoro-3-methyl-phenyl)-[2-(4-trifluoromethyl-phenyl)-ethyl]-amine and tert-butoxycarbonylamino-phenyl-acetic acid. MS (m/e): 431.3 [M+H]⁺.

### EXAMPLE 84

### 2-Amino-N-(3,4-dimethyl-phenyl)-2-phenyl-N-[2-(4-trifluoromethyl-phenyl)-ethyl]-acetamide

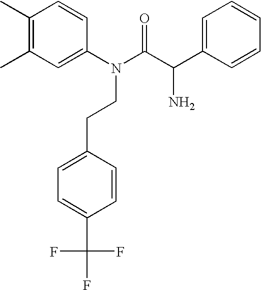

### a) Step 1:

### (3,4-Dimethyl-phenyl)-[2-(4-trifluoromethyl-phenyl)-ethyl]-amine

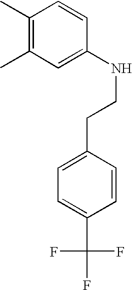

In analogy to the procedure described for the synthesis of example 38 (steps 1 and 2), the title compound was prepared from (3,4-dimethyl-phenyl)-[2-(4-trifluoromethyl-phenyl)-ethyl]-amine and (4-trifluoromethyl-phenyl)-acetic acid MS (m/e): 294.2 [M+H]⁺.

### b) Step 2:

### 2-Amino-N-(3,4-dimethyl-phenyl)-2-phenyl-N-[2-(4-trifluoromethyl-phenyl)-ethyl]-acetamide

In analogy to the procedure described for the synthesis of example 55, the title compound was prepared from (3,4-dimethyl-phenyl)-[2-(4-trifluoromethyl-phenyl)-ethyl]-amine and tert-butoxycarbonylamino-phenyl-acetic acid. MS (m/e): 427.3 [M+H]⁺.

### EXAMPLE 85

### 2-Amino-N-(3,4-dimethoxy-phenyl)-2-(3-fluoro-phenyl)-N-[2-(4-trifluoromethyl-phenyl)-ethyl]-acetamide

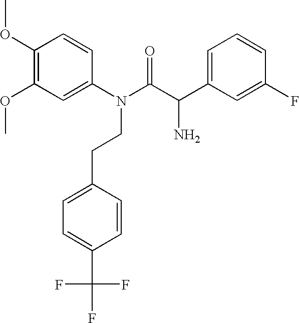

In analogy to the procedure described for the synthesis of example 55, the title compound was prepared from (3,4-dimethoxy-phenyl)-[2-(4-trifluoromethyl-phenyl)-ethyl]-amine (prepared in example 37, steps 1 & 2) and tert-butoxycarbonylamino-(3-fluoro-phenyl)-acetic acid. MS (m/e): 477.3 [M+H]⁺.

### EXAMPLE 86

### 2-Amino-N-(3-chloro-4-methyl-phenyl)-2-phenyl-N-[2-(4-trifluoromethyl-phenyl)-ethyl]-acetamide

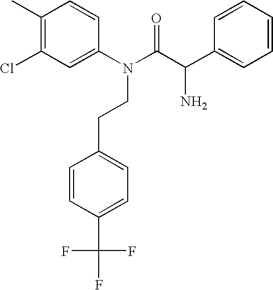

### a) Step 1:

### (3-Chloro-4-methyl-phenyl)-[2-(4-trifluoromethyl-phenyl)-ethyl]-amine

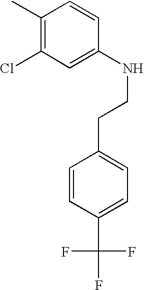

In analogy to the procedure described for the synthesis of example 38 (steps 1 and 2), the title compound was prepared from 3-chloro-4-methyl-phenylamine and (4-trifluoromethyl-phenyl)-acetic acid. This material was used directly for the next step.

### b) Step 2:

### 2-Amino-N-(3-chloro-4-methyl-phenyl)-2-phenyl-N-[2-(4-trifluoromethyl-phenyl)-ethyl]-acetamide

In analogy to the procedure described for the synthesis of example 55, the title compound was prepared from (3-chloro-4-methyl-phenyl)-[2-(4-trifluoromethyl-phenyl)-ethyl]-amine and tert-butoxycarbonylamino-phenyl-acetic acid. MS (m/e): 447.2 [M+H]⁺.

### EXAMPLE 87

### 2-Amino-N-(3-chloro-phenyl)-2-phenyl-N-(2-p-tolyl-ethyl)-acetamide

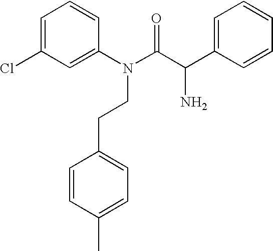

### a) Step 1:

### (3-Chloro-phenyl)-(2-p-tolyl-ethyl)-amine

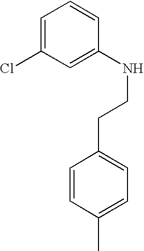

In analogy to the procedure described for the synthesis of example 38 (steps 1 and 2), the title compound was prepared from 3-chloro-phenylamine and (4-trifluoromethyl-phenyl)-acetic acid. This material was used directly for the next step.

### b) Step 2:

### 2-Amino-N-(3-chloro-phenyl)-2-phenyl-N-(2-p-tolyl-ethyl)-acetamide

In analogy to the procedure described for the synthesis of example 55, the title compound was prepared from (3-chloro-phenyl)-(2-p-tolyl-ethyl)-amine and tert-butoxycarbonylamino-phenyl-acetic acid. MS (m/e): 379.2 [M+H]⁺.

### EXAMPLE 88

### 2-Amino-N-(4-fluoro-3-methyl-phenyl)-2-phenyl-N-(2-p-tolyl-ethyl)-acetamide

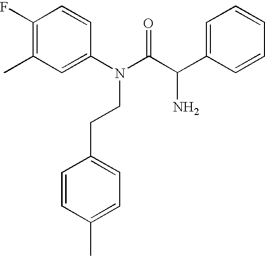

### a) Step 1:

### (4-Fluoro-3-methyl-phenyl)-(2-p-tolyl-ethyl)-amine

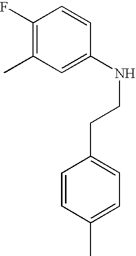

In analogy to the procedure described for the synthesis of example 38 (steps 1 and 2), the title compound was prepared from 4-fluoro-3-methyl-phenylamine and p-tolyl-acetic acid MS (m/e): 244.2 [M+H]⁺.

### b) Step 2:

### 2-Amino-N-(4-fluoro-3-methyl-phenyl)-2-phenyl-N-(2-p-tolyl-ethyl)-acetamide

In analogy to the procedure described for the synthesis of example 55, the title compound was prepared from (4-fluoro-3-methyl-phenyl)-(2-p-tolyl-ethyl)-amine and tert-butoxycarbonylamino-phenyl-acetic acid. MS (m/e): 377.3 [M+H]⁺.

### EXAMPLE 89

### 2-Amino-N-(3,4-dimethyl-phenyl)-2-phenyl-N-(2-p-tolyl-ethyl)-acetamide

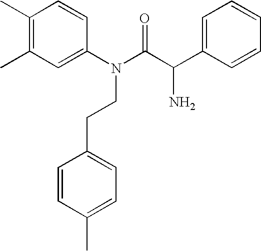

### a) Step 1:

### (3,4-Dimethyl-phenyl)-(2-p-tolyl-ethyl)-amine

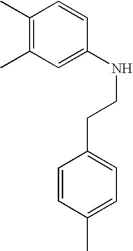

In analogy to the procedure described for the synthesis of example 38 (steps 1 and 2), the title compound was prepared from 3,4-dimethyl-phenylamine and p-tolyl-acetic acid. MS (m/e): 240.4 [M+H]⁺.

### b) Step 2:

### 2-Amino-N-(3,4-dimethyl-phenyl)-2-phenyl-N-(2-p-tolyl-ethyl)-acetamide

In analogy to the procedure described for the synthesis of example 55, the title compound was prepared from (3,4-dimethyl-phenyl)-(2-p-tolyl-ethyl)-amine and tert-butoxycarbonylamino-phenyl-acetic acid. MS (m/e): 373.3 [M+H]⁺.

### EXAMPLE 90

### 2-Amino-N-(3-chloro-phenyl)-N-[2-(4-chloro-phenyl)-ethyl]-2-phenyl-acetamide

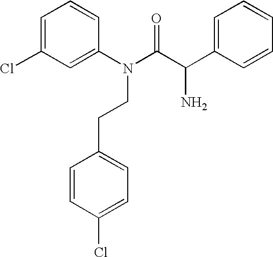

### a) Step 1:

### (3-Chloro-phenyl)-[2-(4-chloro-phenyl)-ethyl]-amine

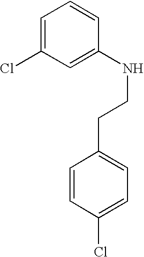

In analogy to the procedure described for the synthesis of example 38 (steps 1 and 2), the title compound was prepared from (4-chloro-phenyl)-acetic acid and 3-chloro-phenylamine. MS (m/e): (no data) [M+H]⁺.

### b) Step 2:

### 2-Amino-N-(3-chloro-phenyl)-N-[2-(4-chloro-phenyl)-ethyl]-2-phenyl-acetamide

In analogy to the procedure described for the synthesis of example 55, the title compound was prepared from (3-chloro-phenyl)-[2-(4-chloro-phenyl)-ethyl]-amine and tert-butoxycarbonylamino-phenyl-acetic acid. MS (m/e): 399.1 [M+H]⁺.

### EXAMPLE 91

### 2-Amino-N-[2-(4-chloro-phenyl)-ethyl]-2-phenyl-N-(3-trifluoromethoxy-phenyl)-acetamide

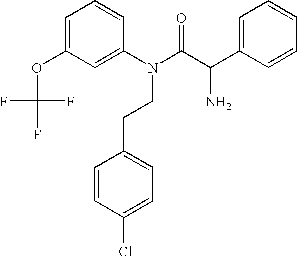

### a) Step 1:

### [2-(4-Chloro-phenyl)-ethyl]-(3-trifluoromethoxy-phenyl)-amine

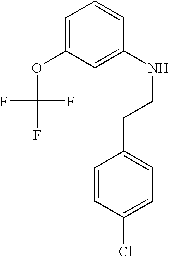

In analogy to the procedure described for the synthesis of example 38 (steps 1 and 2), the title compound was prepared from 3-trifluoromethoxy-phenylamine and (4-chloro-phenyl)-acetic acid. MS (m/e): (no data) [M+H]⁺.

### b) Step 2:

### 2-Amino-N-(3-chloro-phenyl)-N-[2-(4-chloro-phenyl)-ethyl]-2-phenyl-acetamide

In analogy to the procedure described for the synthesis of example 55, the title compound was prepared from [2-(4-chloro-phenyl)-ethyl]-(3-trifluoromethoxy-phenyl)-amine and tert-butoxycarbonylamino-phenyl-acetic acid. MS (m/e): 449.1 [M+H]⁺.

### EXAMPLE 92

### 2-Amino-N-[2-(4-chloro-phenyl)-ethyl]-N-(4-fluoro-3-methyl-phenyl)-2-phenyl-acetamide

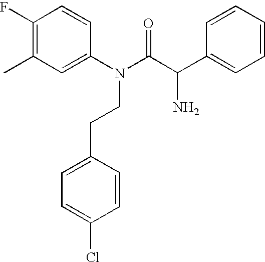

### a) Step 1:

### [2-(4-Chloro-phenyl)-ethyl]-(4-fluoro-3-methyl-phenyl)-amine

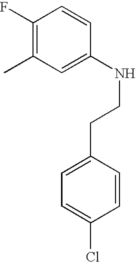

In analogy to the procedure described for the synthesis of example 38 (steps 1 and 2), the title compound was prepared from 4-fluoro-3-methyl-phenylamine and (4-chloro-phenyl)-acetic acid. MS (m/e): (no data) [M+H]⁺.

### b) Step 2:

### 2-Amino-N-[2-(4-chloro-phenyl)-ethyl]-N-(4-fluoro-3-methyl-phenyl)-2-phenyl-acetamide

In analogy to the procedure described for the synthesis of example 55, the title compound was prepared from [2-(4-chloro-phenyl)-ethyl]-(4-fluoro-3-methyl-phenyl)-amine and tert-butoxycarbonylamino-phenyl-acetic acid. MS (m/e): 397.2 [M+H]⁺.

### EXAMPLE 93

### 2-Amino-N-[2-(4-chloro-phenyl)-ethyl]-N-(3,4-dimethyl-phenyl)-2-phenyl-acetamide

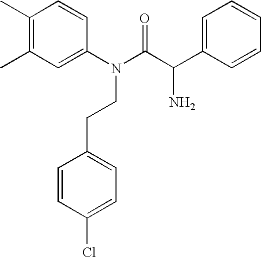

### a) Step 1:

### [2-(4-Chloro-phenyl)-ethyl]-(3,4-dimethyl-phenyl)-amine

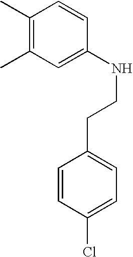

In analogy to the procedure described for the synthesis of example 38 (steps 1 and 2), the title compound was prepared from 3,4-dimethyl-phenylamine and (4-chloro-phenyl)-acetic acid. MS (m/e): (no data) [M+H]⁺.

### b) Step 2:

### 2-Amino-N-[2-(4-chloro-phenyl)-ethyl]-N-(3,4-dimethyl-phenyl)-2-phenyl-acetamide

In analogy to the procedure described for the synthesis of example 55, the title compound was prepared from [2-(4-chloro-phenyl)-ethyl]-(3,4-dimethyl-phenyl)-amine and tert-butoxycarbonylamino-phenyl-acetic acid. MS (m/e): 393.2 [M+H]⁺.

### EXAMPLE 94

### 2-Amino-N-(3,4-difluoro-phenyl)-2-phenyl-N-[2-(4-trifluoromethyl-phenyl)-ethyl]-acetamide

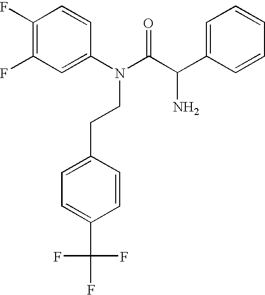

### a) Step 1:

### (3,4-Difluoro-phenyl)-[2-(4-trifluoromethyl-phenyl)-ethyl]-amine

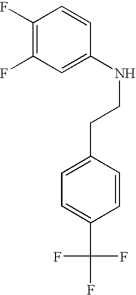

In analogy to the procedure described for the synthesis of example 38 (steps 1 and 2), the title compound was prepared from 3,4-difluoro-phenylamine and (4-trifluoromethyl-phenyl)-acetic acid. MS (m/e): (302.1) [M+H]⁺.

### b) Step 2:

### 2-Amino-N-(3,4-difluoro-phenyl)-2-phenyl-N-[2-(4-trifluoromethyl-phenyl)-ethyl]-acetamide

In analogy to the procedure described for the synthesis of example 55, the title compound was prepared from (3,4-difluoro-phenyl)-[2-(4-trifluoromethyl-phenyl)-ethyl]-amine and tert-butoxycarbonylamino-phenyl-acetic acid. MS (m/e): 435.2 [M+H]⁺.

### EXAMPLE 95

### 2-Amino-N-[3-(3-hydroxy-oxetan-3-yl)-phenyl]-2-phenyl-N-[2-(4-trifluoromethyl-phenyl)-ethyl]-acetamide

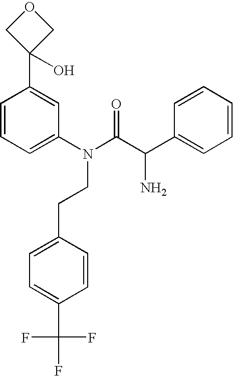

### a) Step 1:

### [3-(3-Hydroxy-oxetan-3-yl)-phenyl]-carbamic acid tert-butyl ester

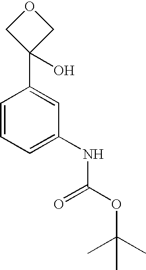

To a solution of (4.53 g, 17 mmol) of (3-bromo-phenyl)-carbamic acid tert-butyl ester in 65 ml dry THF at −78° was added dropwise (21.7 ml) n-BuLi (1.6M in hexane) and the solution stirred at this temp. for 1.5 h. (1.0 g, 14 mmol) of Oxetan-3-one was then added neat to the solution and the resultant solution stirred and warmed to rt over 30 mins then quenched with the addition of satd. aq. NH₄Cl at 0°, and the aqueous phase extracted with EtOAc, dried and concentrated and purified by silica gel chromatography using cyclohexane/EtOAc (1:1) to give a solid which crystallized from Et₂O/n-hexane (66%), m.p. 121-123°, MS (m/e): (264.1) [M+H]⁺ as a white solid.

### b) Step 2:

### 3-(3-Amino-phenyl)-oxetan-3-ol

To a solution of (0.155 g, 0.58 mmol) of [3-(3-hydroxy-oxetan-3-yl)-phenyl]-carbamic acid tert-butyl ester in 1.0 ml of CH₂Cl₂ at rt was added dropwise 0.10 ml of H₃PO₄ and the mixture stirred vigorously for 12 h, diluted with H₂O and then cooled and treated with (7.5 eq) of 4N NaOH. The mixture was then poured onto sat. aq. NaHCO₃, extracted with CH₂Cl₂ and washed with brine, then dried, concentrated and chromatographed on silica gel (using cyclohexane/EtOAc (1:1)) to give the product (41%) as a yellow oil. (m/e): 166.1 [M+H]⁺.

### c) Step 3:

### 3-{3-[2-(4-Trifluoromethyl-phenyl)-ethylamino]-phenyl}-oxetan-3-ol

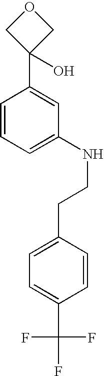

In analogy to the procedure described for the synthesis of example 38 (steps 1 and 2), the title compound was prepared from 3-(3-amino-phenyl)-oxetan-3-ol and (4-trifluoromethyl-phenyl)-acetic acid. MS (m/e): (338.5) [M+H]⁺.

### d) Step 4:

### 2-Amino-N-[3-(3-hydroxy-oxetan-3-yl)-phenyl]-2-phenyl-N-[2-(4-trifluoromethyl-phenyl)-ethyl]-acetamide

In analogy to the procedure described for the synthesis of example 55, the title compound was prepared from 3-{3-[2-(4-trifluoromethyl-phenyl)-ethylamino]-phenyl}-oxetan-3-ol and tert-butoxycarbonylamino-phenyl-acetic acid. MS (m/e): 471.3 [M+H]⁺.

### EXAMPLE 96

### 2-Amino-N-[4-(3-hydroxy-oxetan-3-yl)-phenyl]-2-phenyl-N-[2-(4-trifluoromethyl-phenyl)-ethyl]-acetamide

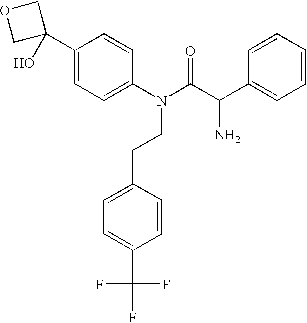

### a) Step 1:

### [4-(3-Hydroxy-oxetan-3-yl)-phenyl]-carbamic acid tert-butyl ester

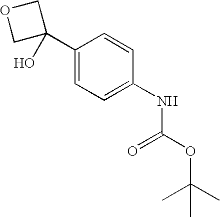

In analogy to the procedure described for the synthesis of example 95 (step 1), the title compound was prepared from oxetan-3-one and (4-bromo-phenyl)-carbamic acid tert-butyl ester. (264.1) [M+H]⁺.

### b) Step 2:

### 3-(4-Amino-phenyl)-oxetan-3-ol

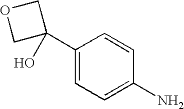

In analogy to the procedure described for the synthesis of example 95 (step 2), the title compound was prepared from. (m/e): 166.1 [M+H]⁺.

### c) Step 3:

### 3-{4-[2-(4-Trifluoromethyl-phenyl)-ethylamino]-phenyl}-oxetan-3-ol

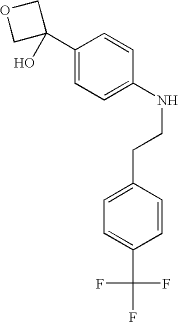

In analogy to the procedure described for the synthesis of example 38 (steps 1 and 2), the title compound was prepared from 3-(4-amino-phenyl)-oxetan-3-ol and (4-trifluoromethyl-phenyl)-acetic acid. MS (m/e): (338.5) [M+H]⁺.

### d) Step 4:

### 2-Amino-N-[4-(3-hydroxy-oxetan-3-yl)-phenyl]-2-phenyl-N-[2-(4-trifluoromethyl-phenyl)-ethyl]-acetamide

In analogy to the procedure described for the synthesis of example 55, the title compound was prepared from 3-{4-[2-(4-trifluoromethyl-phenyl)-ethylamino]-phenyl}-oxetan-3-ol and tert-butoxycarbonylamino-phenyl-acetic acid. MS (m/e): 471.3 [M+H]⁺.

### EXAMPLE 97

### 2-Amino-N-(3,4-dimethoxy-phenyl)-2-(4-trifluoromethoxy-phenyl)-N-[2-(4-trifluoromethyl-phenyl)-ethyl]-acetamide

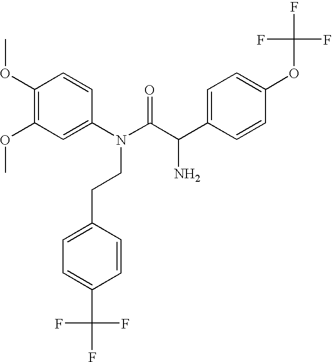

In analogy to the procedure described for the synthesis of example 55, the title compound was prepared from (3,4-dimethoxy-phenyl)-[2-(4-trifluoromethyl-phenyl)-ethyl]-amine (prepared in example 37, steps 1 & 2) and tert-butoxycarbonylamino-(4-trifluoromethoxy-phenyl)-acetic acid. MS (m/e): 543.3 [M+H]⁺.

### EXAMPLE 98

### 2-Amino-N-(3,4-dimethoxy-phenyl)-2-(3-trifluoromethoxy-phenyl)-N-[2-(4-trifluoromethyl-phenyl)-ethyl]-acetamide

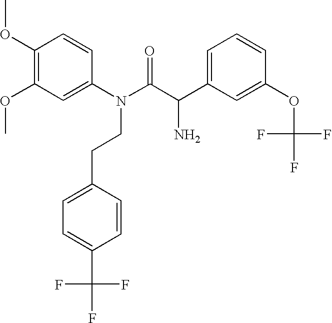

In analogy to the procedure described for the synthesis of example 55, the title compound was prepared from (3,4-dimethoxy-phenyl)-[2-(4-trifluoromethyl-phenyl)-ethyl]-amine (prepared in example 37, steps 1 & 2) and tert-butoxycarbonylamino-(3-trifluoromethoxy-phenyl)-acetic acid MS (m/e): 543.3 [M+H]⁺.

### EXAMPLE 99

### 2-Amino-2-phenyl-N-(3-trifluoromethoxy-phenyl)-N-[2-(4-trifluoromethyl-phenyl)-ethyl]-acetamide

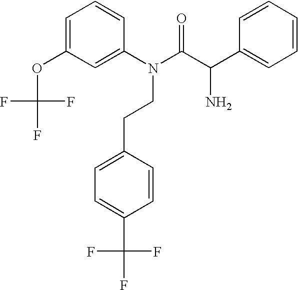

### a) Step 1:

### (3-Trifluoromethoxy-phenyl)-[2-(4-trifluoromethyl-phenyl)-ethyl]-amine

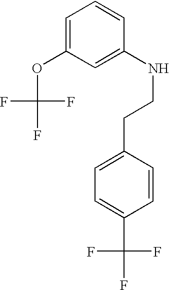

In analogy to the procedure described for the synthesis of example 38 (steps 1 and 2), the title compound was prepared from 3-trifluoromethoxy-phenylamine and (4-trifluoromethyl-phenyl)-acetic acid. MS (m/e): (350.1) [M+H]⁺.

### b) Step 2:

### 2-Amino-2-phenyl-N-(3-trifluoromethoxy-phenyl)-N-[2-(4-trifluoromethyl-phenyl)-ethyl]-acetamide

In analogy to the procedure described for the synthesis of example 55, the title compound was prepared from (3-Trifluoromethoxy-phenyl)-[2-(4-trifluoromethyl-phenyl)-ethyl]-amine and tert-butoxycarbonylamino-phenyl-acetic acid. MS (m/e): 483.2 [M+H]⁺.

### EXAMPLE 100

### (R)—N-(3,4-Dimethoxy-phenyl)-2-(2-methoxy-phenyl)-2-methylamino-N-[2-(4-trifluoromethyl-phenyl)-ethyl]-acetamide hydrochloride

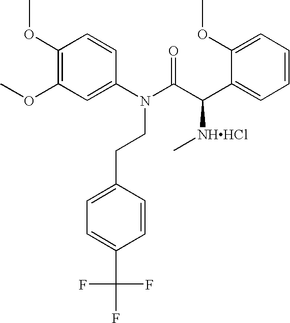

### a) Step 1:

### [{(3,4-Dimethoxy-phenyl)-[2-(4-trifluoromethyl-phenyl)-ethyl]-carbamoyl}-(2-methoxy-phenyl)-methyl]-carbamic acid tert-butyl ester

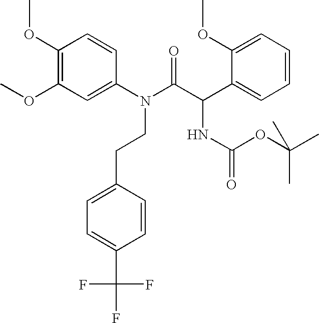

In analogy to the procedure described for the synthesis of example 55 (step 3), the title compound was prepared from (3,4-dimethoxy-phenyl)-[2-(4-trifluoromethyl-phenyl)-ethyl]-amine (prepared in example 37, steps 1 & 2) and tert-butoxycarbonylamino-(2-methoxy-phenyl)-acetic acid MS (m/e): 589.3 [M+H]⁺.

### b) Step 2:

### ({(3,4-Dimethoxy-phenyl)-[2-(4-trifluoromethyl-phenyl)-ethyl]-carbamoyl}-phenyl-methyl)-methyl-carbamic acid tert-butyl ester

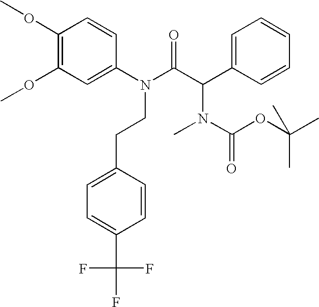

In analogy to the procedure described for the synthesis of example 53, the title compound was prepared from [{(3,4-dimethoxy-phenyl)-[2-(4-trifluoromethyl-phenyl)-ethyl]-carbamoyl}-(2-methoxy-phenyl)-methyl]-carbamic acid tert-butyl ester and MeI. MS (m/e): 573.4 [M+H]⁺.

### c) Step 3:

### (R) —N-(3,4-Dimethoxy-phenyl)-2-(2-methoxy-phenyl)-2-methylamino-N-[2-(4-trifluoromethyl-phenyl)-ethyl]-acetamide hydrochloride

In analogy to the procedure described for the synthesis of example 53 (step 3), the title compound was prepared from ({(3,4-dimethoxy-phenyl)-[2-(4-trifluoromethyl-phenyl)-ethyl]-carbamoyl}-phenyl-methyl)-methyl-carbamic acid tert-butyl ester. The mixture was purified by prep HPLC (least polar isomer, assumed R) to provide the title compound as the free base and thereafter treated with HCl and Et₂O followed by evaporation to give the title compound. MS (m/e): 503.3 [M+H]⁺ (—HCl).

### EXAMPLE 101

### (S)—N-(3,4-Dimethoxy-phenyl)-2-(2-methoxy-phenyl)-2-methylamino-N-[2-(4-trifluoromethyl-phenyl)-ethyl]-acetamide hydrochloride

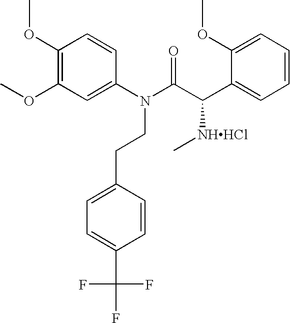

### a) Step 1:

### [{(3,4-Dimethoxy-phenyl)-[2-(4-trifluoromethyl-phenyl)-ethyl]-carbamoyl}-(2-methoxy-phenyl)-methyl]-carbamic acid tert-butyl ester

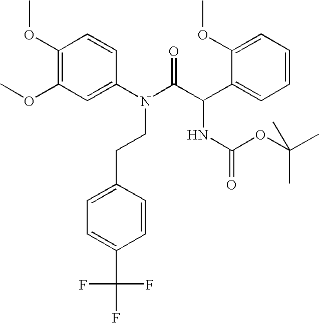

In analogy to the procedure described for the synthesis of example 55 (step 3), the title compound was prepared from (3,4-dimethoxy-phenyl)-[2-(4-trifluoromethyl-phenyl)-ethyl]-amine (prepared in example 37, steps 1 & 2) and tert-butoxycarbonylamino-(2-methoxy-phenyl)-acetic acid MS (m/e): 589.3 [M+H]⁺.

### b) Step 2:

### ({3,4-Dimethoxy-phenyl)-[2-(4-trifluoromethyl-phenyl)-ethyl]-carbamoyl}-phenyl-methyl)-methyl-carbamic acid tert-butyl ester

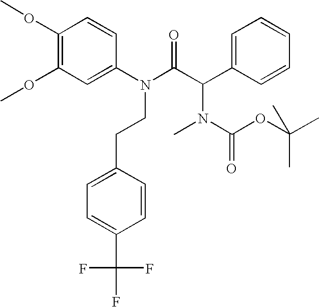

In analogy to the procedure described for the synthesis of example 53 (step 2), the title compound was prepared from [{(3,4-dimethoxy-phenyl)-[2-(4-trifluoromethyl-phenyl)-ethyl]-carbamoyl}-(2-methoxy-phenyl)-methyl]-carbamic acid tert-butyl ester and MeI. MS (m/e): 573.4 [M+H]⁺.

### c) Step 3:

### (S)—N-(3,4-Dimethoxy-phenyl)-2-(2-methoxy-phenyl)-2-methylamino-N-[2-(4-trifluoromethyl-phenyl)-ethyl]-acetamide hydrochloride

In analogy to the procedure described for the synthesis of example 53 (step 3), the title compound was prepared from ({(3,4-dimethoxy-phenyl)-[2-(4-trifluoromethyl-phenyl)-ethyl]-carbamoyl}-phenyl-methyl)-methyl-carbamic acid tert-butyl ester. Purification by chiral HPLC (+ve rotation) to provided the title compound as the free base and thereafter treated with HCl and Et₂O followed by evaporation to give the title compound. MS (m/e): 503.3 [M+H]⁺ (—HCl).

### EXAMPLE 102

### (R)-2-Amino-N-(3,4-dimethoxy-phenyl)-2-(2-methoxy-phenyl)-N-[2-(4-trifluoromethyl-phenyl)-ethyl]-acetamide hydrochloride

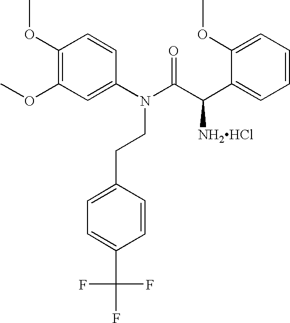

In analogy to the procedure described for the synthesis of example 100 (step 3), the title compound (−ve rotation) was prepared from [{(3,4-dimethoxy-phenyl)-[2-(4-trifluoromethyl-phenyl)-ethyl]-carbamoyl}-(2-methoxy-phenyl)-methyl]-carbamic acid tert-butyl ester (prepared in example 100, step 1). MS (m/e): 489.3 [M+H]⁺ (—HCl).

### EXAMPLE 103

### (S)-2-Amino-N-(3,4-dimethoxy-phenyl)-2-(2-methoxy-phenyl)-N-[2-(4-trifluoromethyl-phenyl)-ethyl]-acetamide hydrochloride

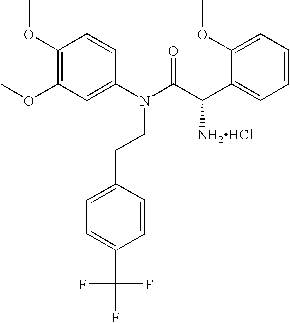

In analogy to the procedure described for the synthesis of example 100 (step 3), the title compound (+ve rotation) was prepared from [{(3,4-dimethoxy-phenyl)-[2-(4-trifluoromethyl-phenyl)-ethyl]-carbamoyl}-(2-methoxy-phenyl)-methyl]-carbamic acid tert-butyl ester (prepared in example 100, step 1). MS (m/e): 489.3 [M+H]⁺ (—HCl).

### EXAMPLE 104

### 2-Amino-N-(4-fluoro-3-methoxy-phenyl)-2-phenyl-N-[2-(4-trifluoromethyl-phenyl)-ethyl]-acetamide

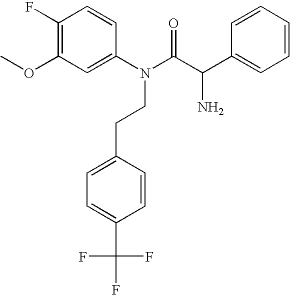

### a) Step 1:

### (4-Fluoro-3-methoxy-phenyl)-[2-(4-trifluoromethyl-phenyl)-ethyl]amine

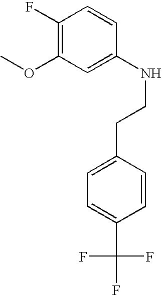

In analogy to the procedure described for the synthesis of example 38 (steps 1 and 2), the title compound was prepared from 4-fluoro-3-methoxy-phenylamine and (4-trifluoromethyl-phenyl)-acetic acid. MS (m/e): (314.2) [M+H]⁺.

### b) Step 2:

### 2-Amino-2-phenyl-N-(3-trifluoromethoxy-phenyl)-N-[2-(4-trifluoromethyl-phenyl)-ethyl]-acetamide

In analogy to the procedure described for the synthesis of example 55, the title compound was prepared from (3-Trifluoromethoxy-phenyl)-[2-(4-trifluoromethyl-phenyl)-ethyl]-amine and tert-butoxycarbonylamino-phenyl-acetic acid. MS (m/e): 447.3 [M+H]⁺.

### EXAMPLE 105

### 2-Amino-N-(4-difluoromethoxy-phenyl)-2-phenyl-N-[2-(4-trifluoromethyl-phenyl)-ethyl]-acetamide

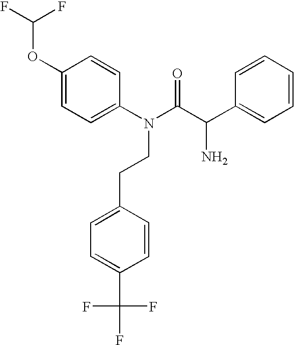

### a) Step 1:

### (4-Difluoromethoxy-phenyl)-[2-(4-trifluoromethyl-phenyl)-ethyl]-amine

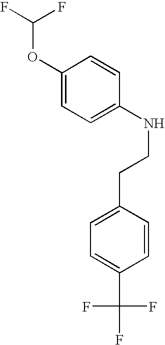

In analogy to the procedure described for the synthesis of example 38 (steps 1 and 2), the title compound was prepared from 4-difluoromethoxy-phenylamine and (4-trifluoromethyl-phenyl)-acetic acid. MS (m/e): (332.1) [M+H]⁺.

### b) Step 2:

### 2-Amino-N-(4-difluoromethoxy-phenyl)-2-phenyl-N-[2-(4-trifluoromethyl-phenyl)-ethyl]-acetamide

In analogy to the procedure described for the synthesis of example 55, the title compound was prepared from (4-Difluoromethoxy-phenyl)-[2-(4-trifluoromethyl-phenyl)-ethyl]-amine and tert-butoxycarbonylamino-phenyl-acetic acid. MS (m/e): 465.0 [M+H]⁺.

### EXAMPLE 106

### N-(3,4-Dimethyl-phenyl)-2-(2-hydroxy-ethylamino)-2-phenyl-N-(2-p-tolyl-ethyl)-acetamide

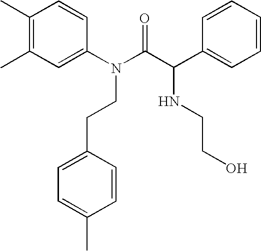

### a) Step 1:

### N-(3,4-Dimethyl-phenyl)-2-oxo-2-phenyl-N-(2-p-tolyl-ethyl)-acetamide

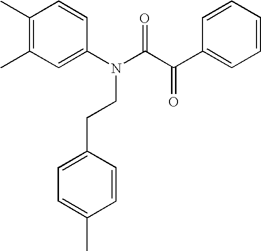

In analogy to the procedure described for the synthesis of example 37 (step 3), the title compound was prepared from (3,4-dimethyl-phenyl)-(2-p-tolyl-ethyl)-amine (prepared in example 89, step 1) and oxo-phenyl-acetic acid. MS (m/e): 372.2 [M+H]⁺.

### b) Step 2:

### N-(3,4-Dimethyl-phenyl)-2-(2-hydroxy-ethylamino)-2-phenyl-N-(2-p-tolyl-ethyl)-acetamide

A solution of 50 mg of (4-difluoromethoxy-phenyl)-[2-(4-trifluoromethyl-phenyl)-ethyl]-amine in 1.0 ml toluene was treated with 9 mg ethanolamine and 32 mg Ti(OEt)₄ and stirred at 100° for 2 h. Thereafter H₂ (2.3 bar), 20 mg 10% Pd/C and 20 uL AcOH was introduced at rt and the sealed mixture stirred at rt for 14 h. Evaporation, redissolution in EtOAc and sequential washing with aq. NaHCO₃ and H₂O followed by concentration of the organic layer and prep. HPLC gave the product as a colourless oil. MS (m/e): 417.3 [M+H]⁺.

### EXAMPLE 107

### 2-Amino-N-(3-difluoromethoxy-phenyl)-2-phenyl-N-[2-(4-trifluoromethyl-phenyl)-ethyl]-acetamide

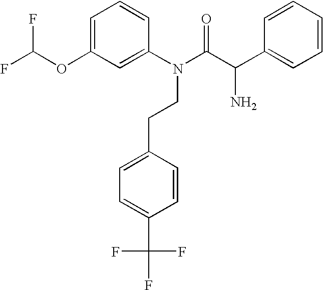

### a) Step 1:

### (3-Difluoromethoxy-phenyl)-[2-(4-trifluoromethyl-phenyl)-ethyl]-amine

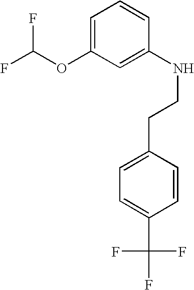

In analogy to the procedure described for the synthesis of example 38 (steps 1 and 2), the title compound was prepared from (3-difluoromethoxy-phenyl)-[2-(4-trifluoromethyl-phenyl)-ethyl]-amine and (4-trifluoromethyl-phenyl)-acetic acid. MS (m/e): (332.2) [M+H]⁺.

### b) Step 2:

### 2-Amino-N-(3-difluoromethoxy-phenyl)-2-phenyl-N-[2-(4-trifluoromethyl-phenyl)-ethyl]-acetamide

In analogy to the procedure described for the synthesis of example 55, the title compound was prepared from (3-difluoromethoxy-phenyl)-[2-(4-trifluoromethyl-phenyl)-ethyl]-amine and tert-butoxycarbonylamino-phenyl-acetic acid. MS (m/e): 465.2 [M+H]⁺.

### EXAMPLE 108

### N-(3,4-Dimethoxy-phenyl)-2-dimethylamino-2-phenyl-N-[2-(4-trifluoromethyl-phenyl)-ethyl]-acetamide

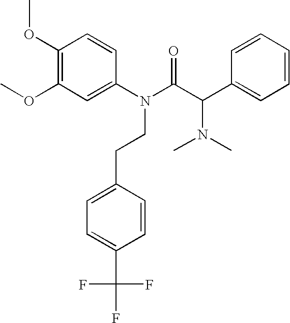

To a solution of 30 mg N-(3,4-dimethoxy-phenyl)-2-methylamino-2-phenyl-N-[2-(4 trifluoromethyl-phenyl)-ethyl]-acetamide in 1.0 ml MeOH at rt was added 6 mg formaldehyde and 4 mg NaCNBH₃ and the mixture stirred at rt for 30 min followed by the addition of 4 mg AcOH and further stirring for 12 h. Evaporation and purification by prep HPLC gave the product as a pale yellow oil. MS (m/e): 487.3 [M+H]⁺.

### EXAMPLE 109

### N-(5-Chloro-2-methoxy-phenyl)-2-hydroxyimino-2-phenyl-N-[2-(4-trifluoromethyl-phenyl)-ethyl]-acetamide

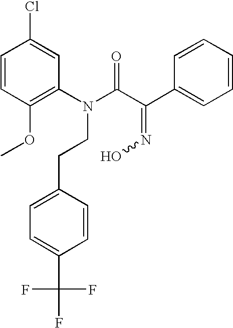

### a) Step 1:

### (5-Chloro-2-methoxy-phenyl)-[2-(4-trifluoromethyl-phenyl)-ethyl]-amine

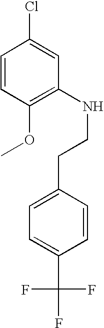

In analogy to the procedure described for the synthesis of example 38 (steps 1 and 2), the title compound was prepared from 5-chloro-2-methoxy-phenylamine and (4-trifluoromethyl-phenyl)-acetic acid. MS (m/e): (330.1) [M+H]⁺.

### b) Step 2:

### N-(5-Chloro-2-methoxy-phenyl)-2-oxo-2-phenyl-N-[2-(4-trifluoromethyl-phenyl)-ethyl]-acetamide

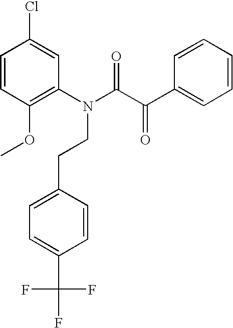

In analogy to the procedure described for the synthesis of example 37 (step 3), the title compound was prepared from (5-chloro-2-methoxy-phenyl)-[2-(4-trifluoromethyl-phenyl)-ethyl]-amine and oxo-phenyl-acetic acid. MS (m/e): 462.2 [M+H]⁺.

### c) Step 3:

### N-(5-Chloro-2-methoxy-phenyl)-2-hydroxyimino-2-phenyl-N-[2-(4-trifluoromethyl-phenyl)-ethyl]-acetamide

A solution of 384 mg of N-(5-chloro-2-methoxy-phenyl)-2-oxo-2-phenyl-N-[2-(4-trifluoromethyl-phenyl)-ethyl]-acetamide in 6.0 ml EtOH was treated with 116 mg HONH₂.HCl and 267 mg 2,6-lutidine and stirred at 53° for 2 d. Evaporation, redissolution in EtOAc and sequential washing with 10% citric acid followed by concentration of the organic layer and prep. HPLC gave the product as a white solid. MS (m/e): 477.0 [M+H]⁺.

### EXAMPLE 110

### 2-Amino-N-(2-chloro-5-methoxy-phenyl)-2-phenyl-N-[2-(4-trifluoromethyl-phenyl)-ethyl]-acetamide

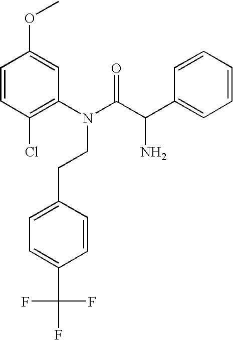

### a) Step 1:

### (2-Chloro-5-methoxy-phenyl)-[2-(4-trifluoromethyl-phenyl)-ethyl]-amine

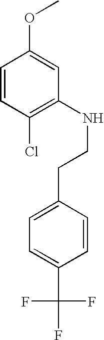

In analogy to the procedure described for the synthesis of example 38 (steps 1 and 2), the title compound was prepared from 2-chloro-5-methoxy-phenylamine and (4-trifluoromethyl-phenyl)-acetic acid. MS (m/e): 330.0 [M+H]⁺.

### b) Step 2:

### 2-Amino-N-(2-chloro-5-methoxy-phenyl)-2-phenyl-N-[2-(4-trifluoromethyl-phenyl)-ethyl]-acetamide

In analogy to the procedure described for the synthesis of example 55, the title compound was prepared from (2-chloro-5-methoxy-phenyl)-[2-(4-trifluoromethyl-phenyl)-ethyl]-amine and tert-butoxycarbonylamino-phenyl-acetic acid. MS (m/e): 463.0 [M+H]⁺.

### EXAMPLE 111

### 2-Amino-N-(5-chloro-2-methoxy-phenyl)-2-phenyl-N-[2-(4-trifluoromethyl-phenyl)-ethyl]-acetamide

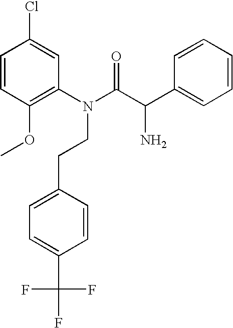

100 mg of N-(5-chloro-2-methoxy-phenyl)-2-hydroxyimino-2-phenyl-N-[2-(4-trifluoromethyl-phenyl)-ethyl]-acetamide (prepared in example 109, step 3) in 2 ml MeOH was treated with 5 mg of 10% Pd/C and 2 eq of TFA and the mixture hydrogenated at 2 bar pressure for 11 h at rt. Filtration, evaporation and redissolution in EtOAc, washing with sat NaHCO₃ and concentration followed by prep hplc gave the title compound (8%) as a colourless oil (as well as the des-Cl analogue described in example 113). MS (m/e): 463.0 [M+H]⁺.

### EXAMPLE 112

### N-(2-Chloro-5-methoxy-phenyl)-2-[(Z)-hydroxyimino]-2-phenyl-N-[2-(4-trifluoromethyl-phenyl)-ethyl]-acetamide

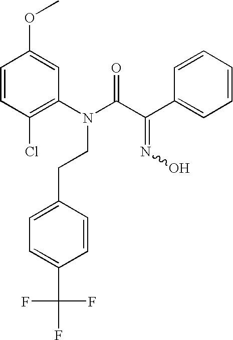

### a) Step 1:

### N-(2-Chloro-5-methoxy-phenyl)-2-oxo-2-phenyl-N-[2-(4-trifluoromethyl-phenyl)-ethyl]-acetamide

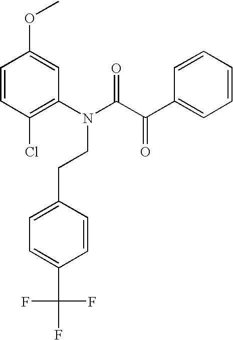

In analogy to the procedure described for the synthesis of example 109 (step 2), the title compound was prepared from (2-chloro-5-methoxy-phenyl)-[2-(4-trifluoromethyl-phenyl)-ethyl]-amine (prepared in example 110, step 1) and oxo-phenyl-acetic acid. MS (m/e): 462.2 [M+H]⁺.

### b) Step 2:

### 2N-(2-chloro-5-methoxy-phenyl)-2-[(Z)-hydroxyimino]-2-phenyl-N-[2-(4-trifluoromethyl-phenyl)-ethyl]-acetamide

In analogy to the procedure described for the synthesis of example 109 (step 3), the title compound was prepared from N-(2-Chloro-5-methoxy-phenyl)-2-oxo-2-phenyl-N-[2-(4-trifluoromethyl-phenyl)-ethyl]-acetamide and HONH₂.HCl. MS (m/e): 477.2 [M+H]⁺.

### EXAMPLE 113

### 2-Amino-N-(2-methoxy-phenyl)-2-phenyl-N-[2-(4-trifluoromethyl-phenyl)-ethyl]-acetamide

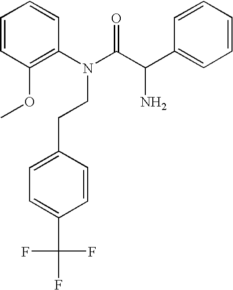

100 mg of N-(5-chloro-2-methoxy-phenyl)-2-hydroxyimino-2-phenyl-N-[2-(4-trifluoromethyl-phenyl)-ethyl]-acetamide (prepared in example 109, step 3) in 2 ml MeOH was treated with 5 mg of 10% Pd/C and 2 eq of TFA and the mixture hydrogenated at 2 bar pressure for 11 h at rt. Filtration, evaporation and redissolution in EtOAc, washing with sat NaHCO₃ and concentration followed by prep hplc gave the title compound (12%) as a colourless oil. MS (m/e): 429.1 [M+H]⁺.

### EXAMPLE 114

### N-(3,4-Dimethyl-phenyl)-2-((S)-3-hydroxy-pyrrolidin-1-yl)-2-phenyl-N-(2-p-tolyl-ethyl)-acetamide

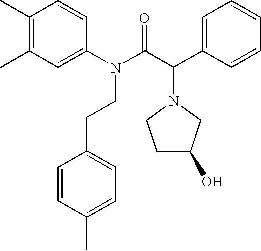

### a) Step 1:

### N-(3,4-Dimethyl-phenyl)-2-hydroxy-2-phenyl-N-(2-p-tolyl-ethyl)-acetamide

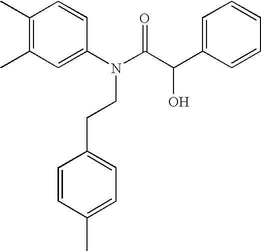

A solution of 800 mg of N-(3,4-dimethyl-phenyl)-2-oxo-2-phenyl-N-(2-p-tolyl-ethyl)-acetamide (prepared in example 106, step 1) in 10 ml MeOH at 0° was treated with 122 mg NaBH₄ and after 15 min warmed at rt and stirred for 14 h. After quenching with 0.5 ml H₂O, evaporation and redissolution in EtOAc, washing with 10% aq. citric acid, filtration and evaporation gave the title compound as a colourless oil. MS (m/e): 374.2 [M+H]⁺.

### b) Step 2:

### Methanesulfonic acid [(3,4-dimethyl-phenyl)-(2-p-tolyl-ethyl)-carbamoyl]-phenyl-methyl ester

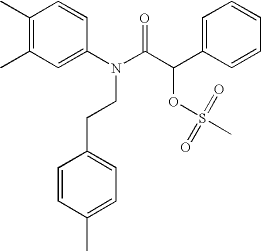

A solution of 640 mg of N-(3,4-dimethyl-phenyl)-2-hydroxy-2-phenyl-N-(2-p-tolyl-ethyl)-acetamide in 10 ml dry CH₂Cl₂ at rt was treated with 206 mg MsCl and 208 mg of Et₃N and stirred for 2 h. Washing with sat. aq. NaHCO₃ and evaporation gave the title compound as a pale yellow oil. MS (m/e): 452.2 [M+H]⁺.

### c) Step 3:

### N-(3,4-Dimethyl-phenyl)-2-((S)-3-hydroxy-pyrrolidin-1-yl)-2-phenyl-N-(2-p-tolyl-ethyl)-acetamide

A solution of 50 mg of methanesulfonic acid [(3,4-dimethyl-phenyl)-(2-p-tolyl-ethyl)-carbamoyl]-phenyl-methyl ester was treated with 3 eq of (S)-pyrrolidin-3-ol, 1.5 eq of Et₃N and 0.2 eq Bu₄NI in 1.0 ml of dry DMF and stirred at 80° for 4 h, cooled and purified by prep. HPLC to give the title compound. MS (m/e): 443.3 [M+H]⁺.

### EXAMPLE 115

### N-(3,4-Dimethyl-phenyl)-2-(4-hydroxy-piperidin-1-yl)-2-phenyl-N-(2-p-tolyl-ethyl)-acetamide

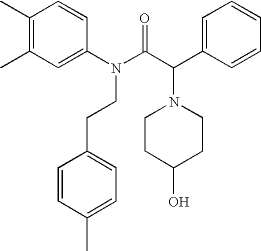

In analogy to the procedure described for the synthesis of example 114, step 2, the title compound was prepared from methanesulfonic acid [(3,4-dimethyl-phenyl)-(2-p-tolyl-ethyl)-carbamoyl]-phenyl-methyl ester (prepared in example 114, step 3) and piperidin-4-ol. MS (m/e): 457.4 [M+H]⁺.

### EXAMPLE 116

### N-(3,4-Dimethyl-phenyl)-2-(2-oxa-6-aza-spiro[3.3]hept-6-yl)-2-phenyl-N-(2-p-tolyl-ethyl)-acetamide

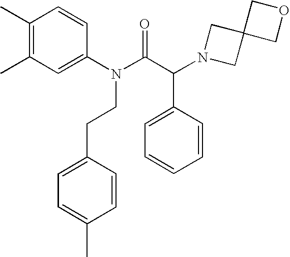

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-oxa-6-aza-spiro[3.3]heptane (prepared as for M. Rogers-Evans et al. “Spirocyclic Oxetanes: Synthesis and Properties; submitted and accepted Angew. Chem., Int. Ed. Engl.,”) MS (m/e): 455.4 [M+H]⁺.

### EXAMPLE 117

### N-(3,4-Dimethyl-phenyl)-2-(3-hydroxy-azetidin-1-yl)-2-phenyl-N-(2-p-tolyl-ethyl)-acetamide

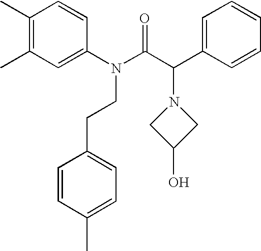

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and azetidin-3-ol. MS (m/e): 429.4 [M+H]⁺.

### EXAMPLE 118

### N-(3,4-Dimethyl-phenyl)-2-((R)-3-hydroxy-pyrrolidin-1-yl)-2-phenyl-N-(2-p-tolyl-ethyl)-acetamide

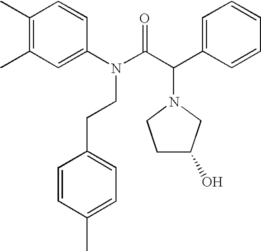

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and (R)-pyrrolidin-3-ol. MS (m/e): 443.4 [M+H]⁺.

### EXAMPLE 119

### ({(3,4-Dimethoxy-phenyl)-[2-(4-trifluoromethyl-phenyl)-ethyl]-carbamoyl}-phenyl-methyl)-carbamic acid tert-butyl ester

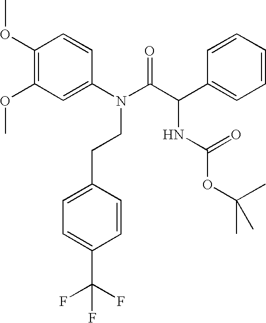

In analogy to the procedure described for the synthesis of example 38, step 3, the title compound was prepared from (3,4-dimethoxy-phenyl)-[2-(4-trifluoromethyl-phenyl)-ethyl]-amine (prepared as for example 37, step 2) and tert-butoxycarbonylamino-(2-methoxy-phenyl)-acetic acid except before the addition of 4 M HCl, the mixture was worked up to give the title compound after prep HPLC. MS (m/e): 559.3 [M+H]⁺.

### EXAMPLE 120

### N-(3,4-Dimethyl-phenyl)-2-(2-methoxy-1-methyl-ethylamino)-2-phenyl-N-(2-p-tolyl-ethyl)-acetamide

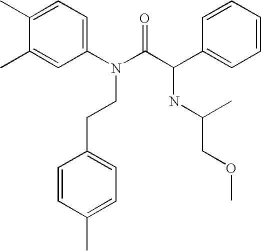

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-methoxy-1-methyl-ethylamine MS (m/e): 445.3 [M+H]⁺.

### EXAMPLE 121

### N-(3,4-Dimethyl-phenyl)-2-(2-hydroxy-propylamino)-2-phenyl-N-(2-p-tolyl-ethyl)-acetamide

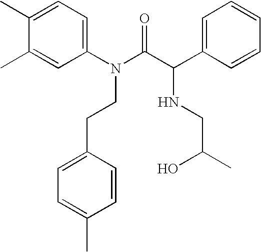

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 1-amino-propan-2-ol. MS (m/e): 431.3 [M+H]⁺.

### EXAMPLE 122

### N-(3,4-Dimethyl-phenyl)-2-(2-hydroxy-1-methyl-ethylamino)-2-phenyl-N-(2-p-tolyl-ethyl)-acetamide

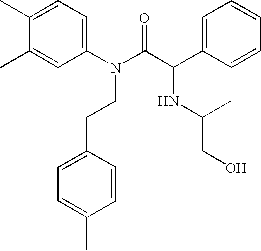

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-amino-propan-1-ol. MS (m/e): 431.3 [M+H]⁺.

### EXAMPLE 123

### N-(3,4-Dimethyl-phenyl)-2-(1-hydroxymethyl-2-methyl-propylamino)-2-phenyl-N-(2-p-tolyl-ethyl)-acetamide

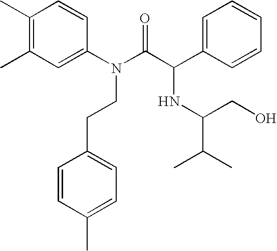

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-amino-3-methyl-butan-1-ol. MS (m/e): 459.4 [M+H]⁺.

### EXAMPLE 124

### 2-(2-Dimethylamino-1-methyl-ethylamino)-N-(3,4-dimethyl-phenyl)-2-phenyl-N-(2-p-tolyl-ethyl)-acetamide

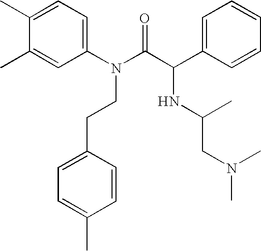

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and N,N′-dimethyl-propane-1,2-diamine. MS (m/e): 458.4 [M+H]⁺.

### EXAMPLE 125

### N-(3,4-Dimethyl-phenyl)-2-(1-methoxymethyl-propylamino)-2-phenyl-N-(2-p-tolyl-ethyl)-acetamide

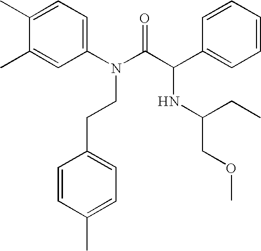

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 1-methoxymethyl-propylamine. MS (m/e): 459.4 [M+H]⁺.

### EXAMPLE 126

### 2-(2-Acetylamino-ethylamino)-N-(3,4-dimethyl-phenyl)-2-phenyl-N-(2-p-tolyl-ethyl)-acetamide

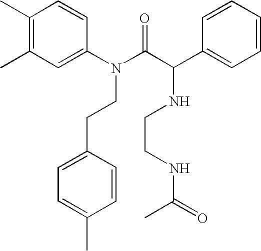

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and N-(2-amino-ethyl)-acetamide. MS (m/e): 458.4 [M+H]⁺.

### EXAMPLE 127

### N-(3,4-Dimethyl-phenyl)-2-phenyl-2-[(tetrahydro-furan-2-ylmethyl)-amino]-N-(2-p-tolyl-ethyl)-acetamide

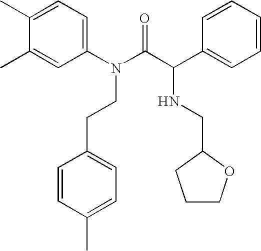

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and (tetrahydro-furan-2-yl)-methylamine. MS (m/e): 457.4 [M+H]⁺.

### EXAMPLE 128

### N-(3,4-Dimethyl-phenyl)-2-isopropylamino-2-phenyl-N-(2-p-tolyl-ethyl)-acetamide

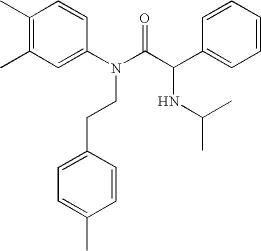

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and isopropylamine. MS (m/e): 415.3 [M+H]⁺.

### EXAMPLE 129

### 2-(2,2-Dimethoxy-ethylamino)-N-(3,4-dimethyl-phenyl)-2-phenyl-N-(2-p-tolyl-ethyl)-acetamide

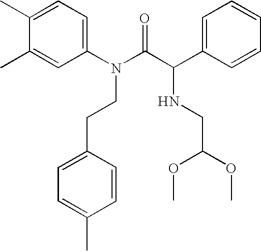

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2,2-dimethoxy-ethylamine. MS (m/e): 461.4 [M+H]⁺.

### EXAMPLE 130

### N-(3,4-Dimethyl-phenyl)-2-[(furan-2-ylmethyl)-amino]-2-phenyl-N-(2-p-tolyl-ethyl)-acetamide

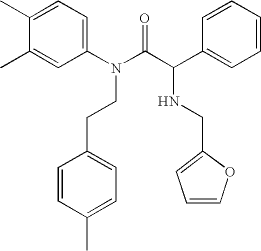

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and furan-2-yl-methylamine. MS (m/e): 453.3 [M+H]⁺.

### EXAMPLE 131

### N-(3,4-Dimethyl-phenyl)-2-(2-hydroxy-butylamino)-2-phenyl-N-(2-p-tolyl-ethyl)-acetamide

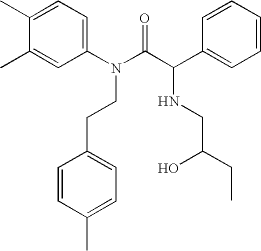

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 1-amino-butan-2-ol. MS (m/e): 445.4 [M+H]⁺.

### EXAMPLE 132

### N-(3,4-Dimethyl-phenyl)-2-(2-hydroxy-1,1-dimethyl-ethylamino)-2-phenyl-N-(2-p-tolyl-ethyl)-acetamide

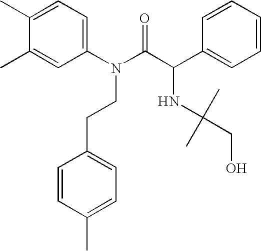

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-amino-2-methyl-propan-1-ol. MS (m/e): 445.4 [M+H]⁺.

### EXAMPLE 133

### N-(3,4-Dimethyl-phenyl)-2-[([1,3]dioxolan-2-ylmethyl)-amino]-2-phenyl-N-(2-p-tolyl-ethyl)-acetamide

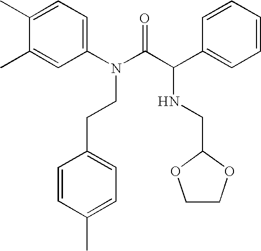

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and [1,3]dioxolan-2-yl-methylamine. MS (m/e): 459.3 [M+H]⁺.

### EXAMPLE 134

### N-(3,4-Dimethyl-phenyl)-2-phenyl-2-{[(S)-1-(tetrahydro-furan-2-yl)methyl]-amino}-N-(2-p-tolyl-ethyl)-acetamide

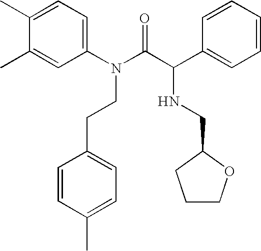

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and [(S)-1-(tetrahydro-furan-2-yl)]-methylamine. MS (m/e): 457.4 [M+H]⁺.

### EXAMPLE 135

### N-(3,4-Dimethyl-phenyl)-2-phenyl-N-(2-p-tolyl-ethyl)-2-(2-vinyloxy-ethylamino)-acetamide

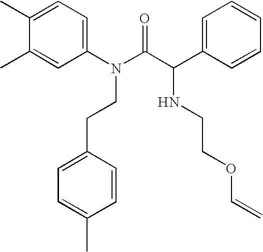

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-vinyloxy-ethylamine. MS (m/e): 443.4 [M+H]⁺.

### EXAMPLE 136

### N-(3,4-Dimethyl-phenyl)-2-(2-ethoxy-ethylamino)-2-phenyl-N-(2-p-tolyl-ethyl)-acetamide

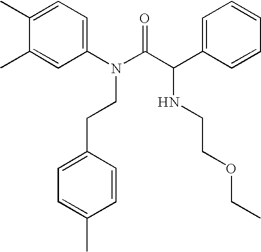

In analogy to the procedure described for the synthesis of example 114, the title compound was prepared from methanesulfonic acid [(3,4-dimethyl-phenyl)-(2-p-tolyl-ethyl)-carbamoyl]-phenyl-methyl ester (prepared in example 114, step 2) and 2-ethoxy-ethylamine. MS (m/e): 445.4 [M+H]⁺.

### EXAMPLE 137

### 2-Amino-2-phenyl-N-(5,6,7,8-tetrahydro-naphthalen-2-yl)-N-[2-(4-trifluoromethyl-phenyl)-ethyl]-acetamide

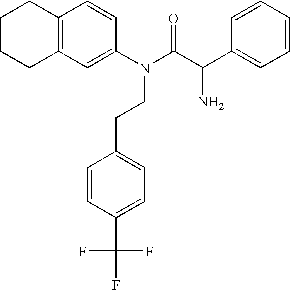

### a) Step 1:

### (5,6,7,8-Tetrahydro-naphthalen-2-yl)-[2-(4-trifluoromethyl-phenyl)-ethyl]-amine

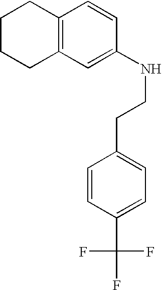

In analogy to the procedure described for the synthesis of example 36, step 1, the title compound was prepared from 5,6,7,8-tetrahydro-naphthalen-2-ylamine and (4-trifluoromethyl)-phenylacetic acid. MS (m/e): 320.2 [M+H]⁺.

### b) Step 2:

### 2-Amino-2-phenyl-N-(5,6,7,8-tetrahydro-naphthalen-2-yl)-N-[2-(4-trifluoromethyl-phenyl)-ethyl]-acetamide

In analogy to the procedure described for the synthesis of example 38, step 3, the title compound was prepared from (5,6,7,8-tetrahydro-naphthalen-2-yl)-[2-(4-trifluoromethyl-phenyl)-ethyl]-amine and tert-butoxycarbonylamino-(2-methoxy-phenyl)-acetic acid. MS (m/e): 453.3 [M+H]⁺.

### EXAMPLE 138

### (S)-2-Amino-N-(3,4-dimethyl-phenyl)-2-phenyl-N-[2-(4-trifluoromethyl-phenyl)-ethyl]-acetamide

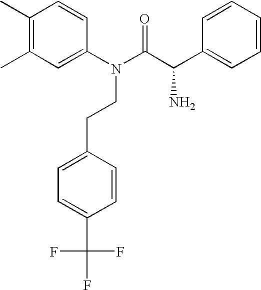

The racemic of product from example 84 was submitted to separation by chiral chromatography to afford the title compound (+ve rotation). MS (m/e): 427.3 [M+H]⁺.

### EXAMPLE 139

### (R)-2-Amino-N-(3,4-dimethyl-phenyl)-2-phenyl-N-[2-(4-trifluoromethyl-phenyl)-ethyl]-acetamide

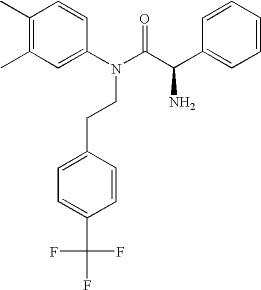

The racemic of product from example 84 was submitted to separation by chiral chromatography to afford the title compound (−ve rotation). MS (m/e): 427.3 [M+H]⁺.

### EXAMPLE 140

### (S)-2-Amino-N-(3,4-diethoxy-phenyl)-2-phenyl-N-[2-(4-trifluoromethyl-phenyl)-ethyl]-acetamide

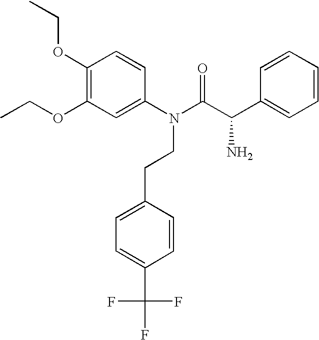

The racemic of product from example 68 was submitted to separation by chiral chromatography to afford the title compound (+ve rotation). MS (m/e): 487.3 [M+H]⁺.

### EXAMPLE 141

### (R)-2-Amino-N-(3,4-diethoxy-phenyl)-2-phenyl-N-[2-(4-trifluoromethyl-phenyl)-ethyl]-acetamide

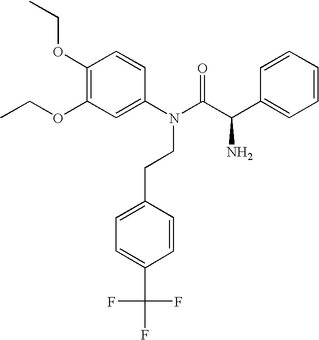

The racemic of product from example 68 was submitted to separation by chiral chromatography to afford the title compound (−ve rotation). MS (m/e): 487.3 [M+H]⁺.

### EXAMPLE 142

### (R)—N-(3,4-Dimethyl-phenyl)-2-hydroxy-2-phenyl-N-(2-p-tolyl-ethyl)-acetamide

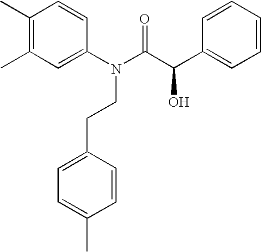

The racemic of product from example 114, step 1 was submitted to separation by chiral chromatography to afford the title compound (−ve rotation). MS (m/e): 374.2 [M+H]⁺.

### EXAMPLE 143

### (S)—N-(3,4-Dimethyl-phenyl)-2-hydroxy-2-phenyl-N-(2-p-tolyl-ethyl)-acetamide

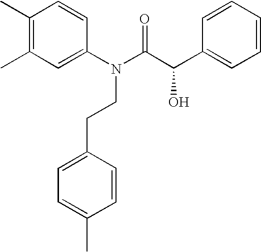

The racemic of product from example 114, step 1 was submitted to separation by chiral chromatography to afford the title compound (+ve rotation). MS (m/e): 374.2 [M+H]⁺.

### EXAMPLE 144

### (S)-2-Amino-N-(3,4-dimethyl-phenyl)-2-(4-fluoro-phenyl)-N-(2-p-tolyl-ethyl)-acetamide

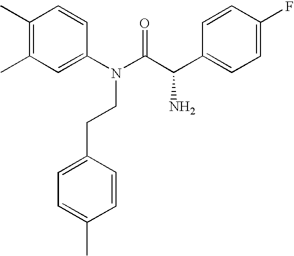

In analogy to the procedure described for the synthesis of example 55, the racemate of the title compound was prepared from (3,4-dimethyl-phenyl)-(2-p-tolyl-ethyl)-amine (prepared in example 89, step 1) and tert-butoxycarbonylamino-(4-fluoro-phenyl)-acetic acid and submitted to separation by chiral chromatography to afford the title compound (+ve rotation). MS (m/e): 391.3 [M+H]⁺.

### EXAMPLE 145

### (R)-2-Amino-N-(3,4-dimethyl-phenyl)-2-(4-fluoro-phenyl)-N-(2-p-tolyl-ethyl)-acetamide

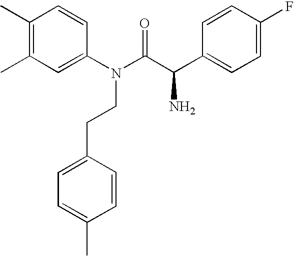

In analogy to the procedure described for the synthesis of example 55, the racemate of the title compound was prepared from (3,4-dimethyl-phenyl)-(2-p-tolyl-ethyl)-amine (prepared in example 89, step 1) and tert-butoxycarbonylamino-(4-fluoro-phenyl)-acetic acid and submitted to separation by chiral chromatography to afford the title compound (−ve rotation). MS (m/e): 391.3 [M+H]⁺.

### EXAMPLE 146

### (S)-2-Amino-2-phenyl-N-p-tolyl-N-[2-(4-trifluoromethyl-phenyl)-ethyl]-acetamide

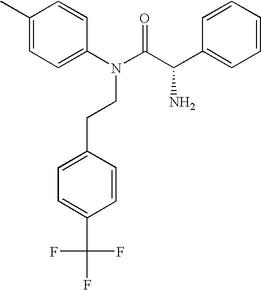

The racemic of product from example 69 was submitted to separation by chiral chromatography to afford the title compound (+ve rotation). MS (m/e): 413.3 [M+H]⁺.

### EXAMPLE 147

### (R)-2-Amino-2-phenyl-N-p-tolyl-N-[2-(4-trifluoromethyl-phenyl)-ethyl]-acetamide

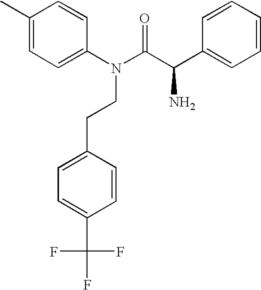

The racemic of product from example 69 was submitted to separation by chiral chromatography to afford the title compound (−ve rotation). MS (m/e): 413.3 [M+H]⁺.

### EXAMPLE 148

### (S)-2-Amino-N-(4-difluoromethoxy-phenyl)-2-phenyl-N-[2-(4-trifluoromethyl-phenyl)-ethyl]-acetamide

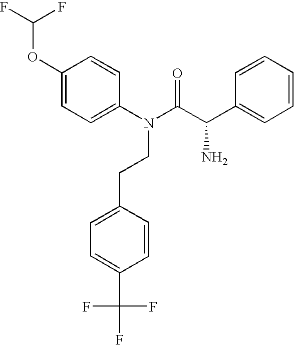

The racemic of product from example 105 was submitted to separation by chiral chromatography to afford the title compound (+ve rotation). MS (m/e): 465.3 [M+H]⁺.

### EXAMPLE 149

### (R)-2-Amino-N-(4-difluoromethoxy-phenyl)-2-phenyl-N-[2-(4-trifluoromethyl-phenyl)-ethyl]-acetamide

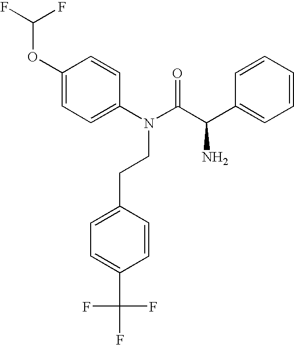

The racemic of product from example 105 was submitted to separation by chiral chromatography to afford the title compound (−ve rotation). MS (m/e): 465.3 [M+H]⁺.

### EXAMPLE 150

### (S)-2-Amino-2-phenyl-N-(5,6,7,8-tetrahydro-naphthalen-2-yl)-N-[2-(4-trifluoromethyl-phenyl)-ethyl]-acetamide

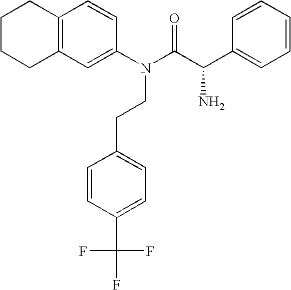

The racemic of product from example 137 was submitted to separation by chiral chromatography to afford the title compound (+ve rotation). MS (m/e): 453.3 [M+H]⁺.

### EXAMPLE 151

### (S)-2-Amino-N-(3-methoxy-phenyl)-2-phenyl-N-[2-(4-trifluoromethyl-phenyl)-ethyl]-acetamide

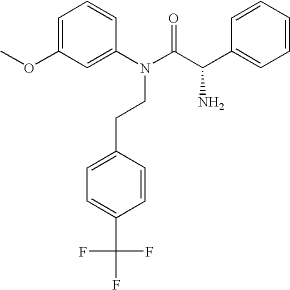

The racemic of product from example 60 was submitted to separation by chiral chromatography to afford the title compound (+ve rotation). MS (m/e): 429.3 [M+H]⁺.

### EXAMPLE 152

### (S)-2-Amino-N-(2-oxo-2,3-dihydro-1H-benzoimidazol-5-yl)-2-phenyl-N-[2-(4-trifluoromethyl-phenyl)-ethyl]-acetamide

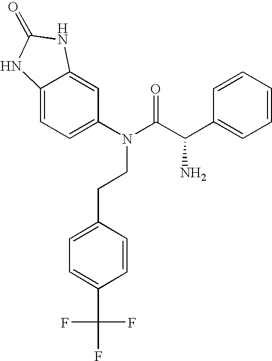

### a) Step 1:

### 5-[2-(4-Trifluoromethyl-phenyl)-ethylamino]-1,3-dihydro-benzoimidazol-2-one

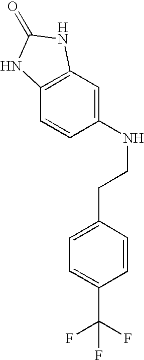

A solution of 3.0 mmol (4-trifluoromethyl-phenyl)-acetonitrile and 2.0 mmol 5-amino-1,3-dihydro-benzoimidazol-2-one in MeOH (10 ml) was treated with NH₄OAc (12.00 mmol) and 10% Pd/C (200 mg) and stirred at rt for 72 h. Filtration, concentration and purification by chromatography (SiO₂, heptane:ethyl acetate=95:5 to 60:40) afforded the title compound (73%) and was used directly for the next step.

### b) Step 2:

In analogy to the procedure described for the synthesis of example 38 (step 3), the title compound was prepared from 5-[2-(4-trifluoromethyl-phenyl)-ethylamino]-1,3-dihydro-benzoimidazol-2-one and (S)-tert-butoxycarbonylamino-phenyl-acetic acid, MS (m/e): 455.1 [M+H]⁺.

### EXAMPLE 153

### (R)-2-Amino-N-(3-methoxy-phenyl)-2-phenyl-N-[2-(4-trifluoromethyl-phenyl)-ethyl]-acetamide

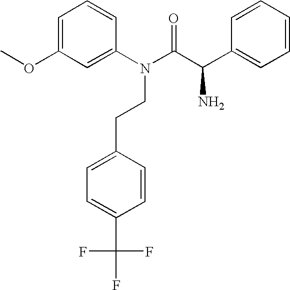

The racemic of product from example 60 was submitted to separation by chiral chromatography to afford the title compound (−ve rotation). MS (m/e): 429.3 [M+H]⁺.

### EXAMPLE 154

### (S)-2-Amino-N-(3,4-dimethyl-phenyl)-2-(4-fluoro-phenyl)-N-[2-(4-trifluoromethyl-phenyl)-ethyl]-acetamide

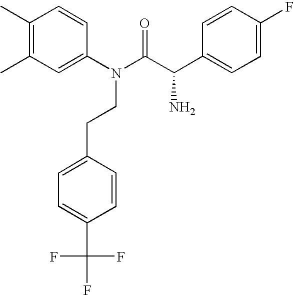

In analogy to the procedure described for the synthesis of example 55, the racemate of the title compound was prepared from (3,4-dimethyl-phenyl)-[2-(4-trifluoromethyl-phenyl)-ethyl]-amine (prepared in example 84, step 1) and tert-butoxycarbonylamino-(4-fluoro-phenyl)-acetic acid and submitted to separation by chiral chromatography to afford the title compound (+ve rotation). MS (m/e): 445.3 [M+H]⁺.

### EXAMPLE 155

### (R)-2-Amino-N-(3,4-dimethyl-phenyl)-2-(4-fluoro-phenyl)-N-[2-(4-trifluoromethyl-phenyl)-ethyl]-acetamide

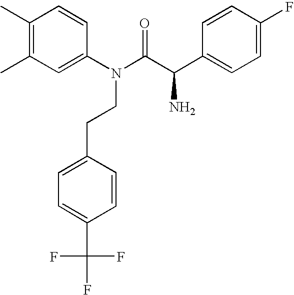

In analogy to the procedure described for the synthesis of example 55, the racemate of the title compound was prepared from (3,4-dimethyl-phenyl)-[2-(4-trifluoromethyl-phenyl)-ethyl]-amine (prepared in example 84, step 1) and tert-butoxycarbonylamino-(4-fluoro-phenyl)-acetic acid and submitted to separation by chiral chromatography to afford the title compound (−ve rotation). MS (m/e): 445.3 [M+H]⁺.

### EXAMPLE 156

### (R)—N-(3,4-Dimethyl-phenyl)-2-(2-methoxy-phenyl)-2-methylamino-N-[2-(4-trifluoromethyl-phenyl)-ethyl]-acetamide

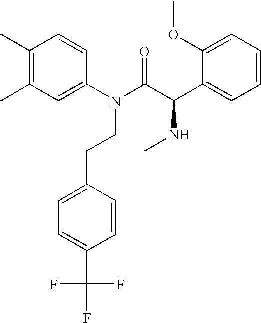

### a) Step 1:

### [{(3,4-Dimethyl-phenyl)-[2-(4-trifluoromethyl-phenyl)-ethyl]-carbamoyl}-(2-methoxy-phenyl)-methyl]-carbamic acid tert-butyl ester

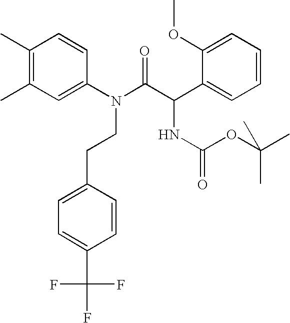

In analogy to the procedure described for the synthesis of example 55, the title compound was prepared from (3,4-dimethyl-phenyl)-[2-(4-trifluoromethyl-phenyl)-ethyl]-amine (prepared in example 84, step 1) and tert-butoxycarbonylamino-(2-methoxy-phenyl)-acetic acid MS (m/e): 557.4 [M+H]⁺.

### b) Step 2:

### (R)—N-(3,4-Dimethyl-phenyl)-2-(2-methoxy-phenyl)-2-methylamino-N-[2-(4-trifluoromethyl-phenyl)-ethyl]-acetamide

In analogy to the procedure described for the synthesis of example 53 (steps 2 & 3), the title compound was prepared from [{(3,4-dimethyl-phenyl)-[2-(4-trifluoromethyl-phenyl)-ethyl]-carbamoyl}-(2-methoxy-phenyl)-methyl]-carbamic acid tert-butyl ester and submitted to separation by chiral chromatography to afford the title compound (−ve rotation). MS (m/e): 471.3 [M+H]⁺.

### EXAMPLE 157

### (S)—N-(3,4-Dimethyl-phenyl)-2-(2-methoxy-phenyl)-2-methylamino-N-[2-(4-trifluoromethyl-phenyl)-ethyl]-acetamide

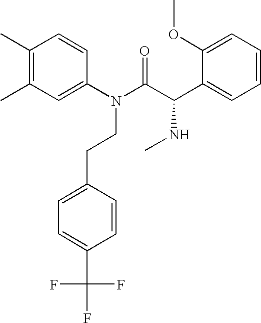

The racemate from example 156, step 2 was submitted to separation by chiral chromatography to afford the title compound (+ve rotation). MS (m/e): 471.3 [M+H]⁺.

### EXAMPLE 158

### (R)—N-(3,4-Dimethyl-phenyl)-2-hydroxy-2-phenyl-N-[2-(4-trifluoromethyl-phenyl)-ethyl]-acetamide

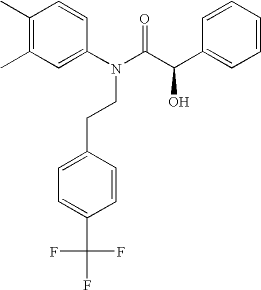

In analogy to example 114, step 2, N-(3,4-dimethyl-phenyl)-2-oxo-2-phenyl-N-[2-(4-trifluoromethyl-phenyl)-ethyl]-acetamide from example 218 was reduced and submitted to separation by chiral chromatography to afford the title compound (−ve rotation). MS (m/e): 428.3 [M+H]⁺.

### EXAMPLE 159

### (S)—N-(3,4-Dimethyl-phenyl)-2-hydroxy-2-phenyl-N-[2-(4-trifluoromethyl-phenyl)-ethyl]-acetamide

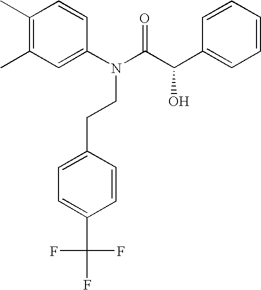

In analogy to example 114, step 2, N-(3,4-dimethyl-phenyl)-2-oxo-2-phenyl-N-[2-(4-trifluoromethyl-phenyl)-ethyl]-acetamide from example 218 was reduced and submitted to separation by chiral chromatography to afford the title compound (+ve rotation). MS (m/e): 428.3 [M+H]⁺.

### EXAMPLE 160

### (S)-2-Amino-N-(3,4-dimethyl-phenyl)-2-(4-fluoro-phenyl)-N-[2-(4-trifluoromethyl-phenyl)-ethyl]-acetamide

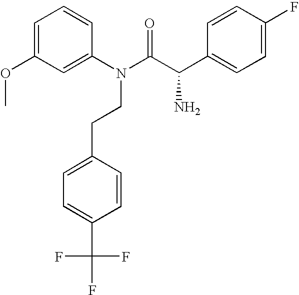

In analogy to the procedure described for the synthesis of example 55, the racemate of the title compound was prepared from (3-methoxy-phenyl)-[2-(4-trifluoromethyl-phenyl)-ethyl]-amine (prepared in example 38, step 2) and tert-butoxycarbonylamino-(4-fluoro-phenyl)-acetic acid and submitted to separation by chiral chromatography to afford the title compound (+ve rotation). MS (m/e): 447.2 [M+H]⁺.

### EXAMPLE 161

### (S)—N-(3,4-Dimethyl-phenyl)-2-(4-fluoro-phenyl)-2-hydroxy-N-[2-(4-trifluoromethyl-phenyl)-ethyl]-acetamide

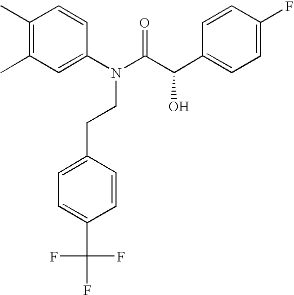

### a) Step 1:

### N-(3,4-Dimethyl-phenyl)-2-(4-fluoro-phenyl)-2-oxo-N-[2-(4-trifluoromethyl-phenyl)-ethyl]-acetamide

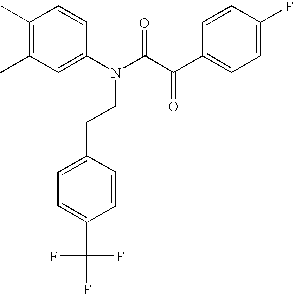

In analogy to example 106, step 1, (3,4-dimethyl-phenyl)-[2-(4-trifluoromethyl-phenyl)-ethyl]-amine (prepared in example 84, step 1) was coupled to (4-fluoro-phenyl)-oxo-acetic acid to afford the title compound and used directly for the next step.

### b) Step 2:

### (S)—N-(3,4-Dimethyl-phenyl)-2-(4-fluoro-phenyl)-2-hydroxy-N-[2-(4-trifluoromethyl-phenyl)-ethyl]-acetamide

In analogy to example 114, step 1, N-(3,4-dimethyl-phenyl)-2-(4-fluoro-phenyl)-2-oxo-N-[2-(4-trifluoromethyl-phenyl)-ethyl]-acetamide was reduced and submitted to separation by chiral chromatography to afford the title compound (+ve rotation). MS (m/e): 446.2 [M#H]⁺.

### EXAMPLE 162

### (S)-2-Amino-N-(3-methoxy-4-methyl-phenyl)-2-phenyl-N-[2-(4-trifluoromethyl-phenyl)-ethyl]-acetamide

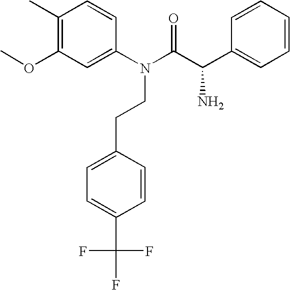

In analogy to the procedure described for the synthesis of example 38 (steps 1, 2 & 3), the title compound was prepared from 3-methoxy-4-methyl-phenylamine, (4-trifluoromethyl-phenyl)-acetic acid and (S)-tert-butoxycarbonylamino-phenyl-acetic acid. MS (m/e): 443.1 [M#H]⁺.

### EXAMPLE 163

### (S)-2-Amino-N-(3-chloro-4-difluoromethoxy-phenyl)-2-(4-fluoro-phenyl)-N-[2-(4-trifluoromethyl-phenyl)-ethyl]-acetamide

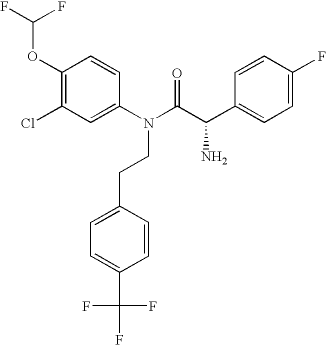

### a) Step 1:

### (3-Chloro-4-difluoromethoxy-phenyl)-[2-(4-trifluoromethyl-phenyl)-ethyl]-amine

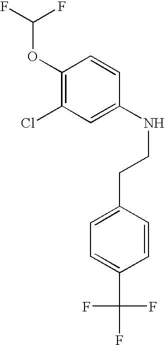

In analogy to the procedure described for the synthesis of example 37 (steps 1 and 2), the title compound was prepared from 3-chloro-4-difluoromethoxy-phenylamine (4-trifluoromethyl-phenyl)-acetic acid. MS (m/e): 378.1 [M+H]⁺.

### b) Step 2:

### (S)-2-Amino-N-(3-chloro-4-difluoromethoxy-phenyl)-2-(4-fluoro-phenyl)-N-[2-(4-trifluoromethyl-phenyl)-ethyl]-acetamide

In analogy to the procedure described for the synthesis of example 55 (step 3), the racemate of the title compound was prepared from (3-chloro-4-difluoromethoxy-phenyl)-[2-(4-trifluoromethyl-phenyl)-ethyl]-amine and tert-butoxycarbonylamino-(4-fluoro-phenyl)-acetic acid and submitted to separation by chiral chromatography to afford the title compound (+ve rotation). MS (m/e): 517.2 [M+H]⁺.

### EXAMPLE 164

### (R)-2-Amino-N-(3-chloro-4-difluoromethoxy-phenyl)-2-(4-fluoro-phenyl)-N-[2-(4-trifluoromethyl-phenyl)-ethyl]-acetamide

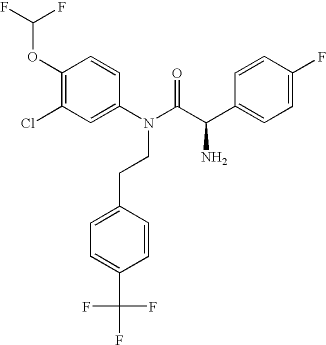

The racemic product from example 163, step 2 was submitted to separation by chiral chromatography to afford the title compound (−ve rotation). MS (m/e): 517.2 [M+H]⁺.

### EXAMPLE 165

### (S)-2-Amino-N-(3-chloro-4-ethoxy-phenyl)-2-(4-fluoro-phenyl)-N-[2-(4-trifluoromethyl-phenyl)-ethyl]-acetamide

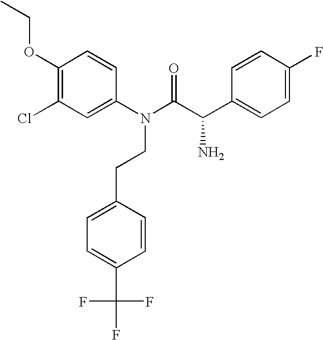

### a) Step 1:

### (3-Chloro-4-ethoxy-phenyl)-[2-(4-trifluoromethyl-phenyl)-ethyl]-amine

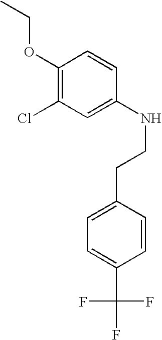

In analogy to the procedure described for the synthesis of example 37 (steps 1 and 2), the title compound was prepared from 3-chloro-4-ethoxy-phenylamine and (4-trifluoromethyl-phenyl)-acetic acid and used directly for the next step.

### b) Step 2:

### (S)-2-Amino-N-(3-chloro-4-ethoxy-phenyl)-2-(4-fluoro-phenyl)-N-[2-(4-trifluoromethyl-phenyl)-ethyl]-acetamide

In analogy to the procedure described for the synthesis of example 55, the racemate of the title compound was prepared from (3-chloro-4-difluoromethoxy-phenyl)-[2-(4-trifluoromethyl-phenyl)-ethyl]-amine and tert-butoxycarbonylamino-(4-fluoro-phenyl)-acetic acid and submitted to separation by chiral chromatography to afford the title compound (+ve rotation). MS (m/e): 495.2 [M+H]⁺.

### EXAMPLE 166

### (R)-2-Amino-N-(3-chloro-4-ethoxy-phenyl)-2-(4-fluoro-phenyl)-N-[2-(4-trifluoromethyl-phenyl)-ethyl]-acetamide

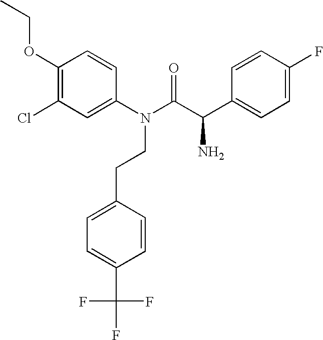

The racemate of the product from example 165 was submitted to separation by chiral chromatography to afford the title compound (−ve rotation). MS (m/e): 495.2 [M+H]⁺.

### EXAMPLE 167

### (S)-2-Acetylamino-N-(3,4-dimethyl-phenyl)-2-phenyl-N-(2-p-tolyl-ethyl)-acetamide

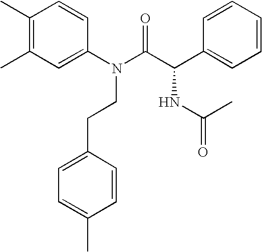

To a solution of 448 mg of 2-amino-N-(3,4-dimethyl-phenyl)-2-phenyl-N-(2-p-tolyl-ethyl)-acetamide (89, step 2) in 10 ml CH₂Cl₂ at RT was added 3 eq. Et₃N then 1.05 eq. Ac₂O and stirred for 2 h. The mixture was washed with 2N NaHCO₃, H₂0, dried and evaporated, then chromatographed on silica (EtOAc/Heptane gradient elution) to give a pale yellow oil which was submitted to separation by chiral chromatography to afford the title compound (+ve rotation). MS (m/e): 415.2 [M+H]⁺.

### EXAMPLE 168

### ([({[(3,4-Dimethyl-phenyl)-(2-p-tolyl-ethyl)-carbamoyl]-phenyl-methyl}-carbamoyl)-methyl]-carbamic acid tert-butyl ester

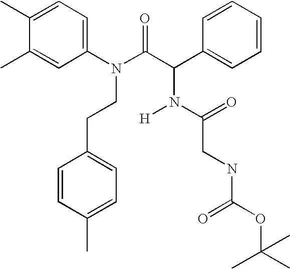

To a 0° C. solution of 0.25 g tert-butoxycarbonylamino-acetic acid in 10 ml dichloromethane were added successively 1.05 eq 1-(3-dimethylaminopropyl)-3-ethylcarbodiimide hydrochloride, 1 eq 2-amino-N-(3,4-dimethyl-phenyl)-2-phenyl-N-(2-p-tolyl-ethyl)-acetamide (from example 89, step 2). The mixture was stirred at 0° C. for 30 minutes and then at room temperature for 30 minutes. The solution was washed with a sat. NaHCO₃ solution and with water, dried over Na₂SO₄, filtered and concentrated in vacuo. The crude solid was purified with flash column chromatography on silica eluting with a gradient formed from heptane and ethylacetate to provide the title compound. MS (m/e): 530.3 [M+H]⁺.

### EXAMPLE 169

### N-{[(3,4-Dimethyl-phenyl)-(2-p-tolyl-ethyl)-carbamoyl]-phenyl-methyl}-propionamide

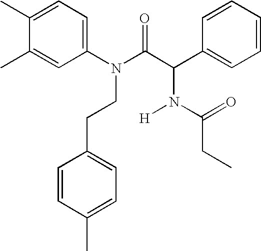

In analogy to the procedure described for the synthesis of example 168, the title compound was prepared from 2-amino-N-(3,4-dimethyl-phenyl)-2-phenyl-N-(2-p-tolyl-ethyl)-acetamide (from example 89, step 2) and propionic acid to afford the title compound. MS (m/e): 429.2 [M+H]⁺.

### EXAMPLE 170

### N-{[(3,4-Dimethyl-phenyl)-(2-p-tolyl-ethyl)-carbamoyl]-phenyl-methyl}-propionamide

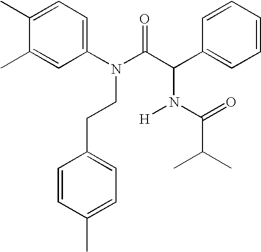

In analogy to the procedure described for the synthesis of example 168, the title compound was prepared from 2-amino-N-(3,4-dimethyl-phenyl)-2-phenyl-N-(2-p-tolyl-ethyl)-acetamide (89, step 2) and iso-Butyric acid to afford the title compound. MS (m/e): 443.3 [M+H]⁺.

### EXAMPLE 171

### N-(3,4-Dimethyl-phenyl)-2-formylamino-2-phenyl-N-(2-p-tolyl-ethyl)-acetamide

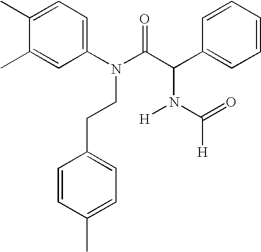

In analogy to the procedure described for the synthesis of example 168, the title compound was prepared from 2-amino-N-(3,4-dimethyl-phenyl)-2-phenyl-N-(2-p-tolyl-ethyl)-acetamide (from example 89, step 2) and formic acid to afford the title compound. MS (m/e): 401.2 [M+H]⁺.

### EXAMPLE 172

### [(S)-1-({[(3,4-Dimethyl-phenyl)-(2-p-tolyl-ethyl)-carbamoyl]-phenyl-methyl}-carbamoyl)-ethyl]-carbamic acid tert-butyl ester

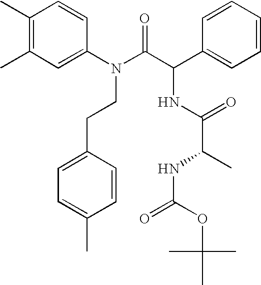

In analogy to the procedure described for the synthesis of example 168, the title compound was prepared from 2-amino-N-(3,4-dimethyl-phenyl)-2-phenyl-N-(2-p-tolyl-ethyl)-acetamide (from example 89, step 2) and t-Boc-alanine to afford the title compound. MS (m/e): 544.2 [M+H]⁺.

### EXAMPLE 173

### (S)-2-Amino-N-(3,4-dimethoxy-phenyl)-2-phenyl-N-[2-(4-trifluoromethyl-phenyl)-ethyl]-acetamide hydrochloride

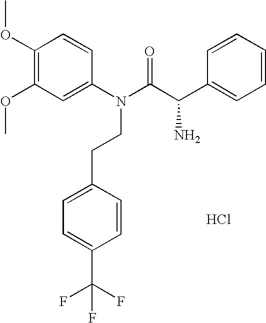

### a) Step 1:

### N-(3,4-Dimethoxy-phenyl)-2-(4-trifluoromethyl-phenyl)-acetamide

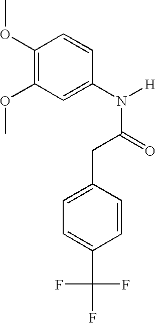

To a 0° C. solution of 7.1 g (34 mmol) 4-(trifluoromethyl)phenylacetic acid in 150 ml dichloromethane were added successively 5 g (32 mmol) 3,4-dimethoxyaniline and 6.6 g (34 mmol) 1-(3-dimethylaminopropyl)-3-ethylcarbodiimide hydrochloride. The mixture was stirred at 0° C. for 30 minutes and then at room temperature for 30 minutes. The solution was washed with a sat. NaHCO₃ solution and with water, dried over Na₂SO₄, filtered and concentrated in vacuo. The crude solid was purified with flash column chromatography on silica eluting with a gradient formed from heptane and ethylacetate to provide 8.7 g (80%) of the title compound as an off-white solid. MS (m/e): 340.4 [M+H]⁺.

### b) Step 2:

### (3,4-Dimethoxy-phenyl)-[2-(4-trifluoromethyl-phenyl)-ethyl]-amine

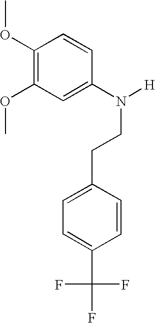

To a solution of 8.7 g (26 mmol) N-(3,4-dimethoxy-phenyl)-2-(4-trifluoromethyl-phenyl)-acetamide in 175 ml THF under argon at room temperature, was added dropwise 51.3 ml (51.3 mmol) of a 1 M borane-tetrahydrofuran solution. The solution was refluxed for 3 hours, cooled to 0° C. and quenched with 120 ml of a 20% NH₄Cl solution. The organic layer was separated and the aqueous layer was extracted once with ethyl acetate. The combined extracts were concentrated in vacuo. The residue was dissolved in 100 ml methanol. The solution was acidified with HCl 5N and stirred at room temperature for 1.5 hours. The solution was basified with a sat. NaHCO₃ solution, and concentrated. The residue was dissolved in ethylacetate, the aqueous phase was extracted 3 times with ethyl acetate. The combined extracts were dried over Na₂SO₄, filtered and concentrated in vacuo. The crude oil was purified with flash column chromatography on silica eluting with a gradient formed from heptane and ethylacetate to provide 7.1 g (85%) of the title compound as a colorless oil. MS (m/e): 326.4 [M+H]⁺.

### c) Step 3:

### ((S)-{(3,4-Dimethoxy-phenyl)-[2-(4-trifluoromethyl-phenyl)-ethyl]-carbamoyl}-phenyl-methyl)-carbamic acid tert-butyl ester

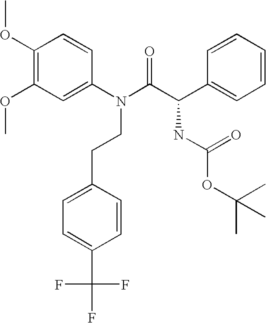

To a 0° C. solution of 3.25 g (10 mmol) (3,4-dimethoxy-phenyl)-[2-(4-trifluoromethyl-phenyl)-ethyl]-amine and 2.8 g (11 mmol) Boc-L-alpha-phenylglycine in 50 ml dichloromethane under argon, was added 2.1 g (11 mmol) 1-(3-dimethylaminopropyl)-3-ethylcarbodiimide hydrochloride. The mixture was stirred at 0° C. for 4 hours. The solution was washed once with 50 ml of a sat. NaHCO₃ solution and once with 50 ml water. The combined extracts were dried over Na₂SO₄, filtered and concentrated in vacuo. The crude oil was purified with flash column chromatography on silica eluting with a gradient formed from heptane and ethylacetate to provide 5.6 g (100%) of the title compound as a yellow gum. MS (m/e): 558.8 [M+H]⁺.

### d) Step 4:

### (S)-2-Amino-N-(3,4-dimethoxy-phenyl)-2-phenyl-N-[2-(4-trifluoromethyl-phenyl)-ethyl]-acetamide hydrochloride

To a solution of 5.6 g (10 mmol) ((S)-{(3,4-Dimethoxy-phenyl)-[2-(4-trifluoromethyl-phenyl)-ethyl]-carbamoyl}-phenyl-methyl)-carbamic acid tert-butyl ester in 22.6 mL dioxane was added 25 ml (100 mmol) of a 4 M HCl solution in dioxane. The mixture was stirred at room temperature for 18 hours. The solvent was removed in vacuo. Ethylacetate was added and the mixture was stirred slowly at ambient temperature. The solid was filtered, rinsed with ether and dried under vacuum to provide 4.6 g (92%) of the title compound as a light yellow solid MS (m/e): 459.3 [M+H]⁺.

### EXAMPLE 174

### (R)-2-Amino-N-(3,4-dimethoxy-phenyl)-2-phenyl-N-[2-(4-trifluoromethyl-phenyl)-ethyl]-acetamide

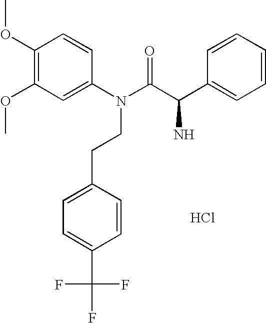

### a) Step 1:

### ({(3,4-Dimethoxy-phenyl)-[2-(4-trifluoromethyl-phenyl)-ethyl]-carbamoyl}-phenyl-methyl)-carbamic acid tert-butyl ester

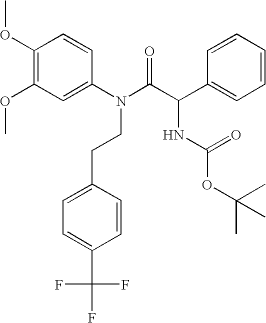

In analogy to the procedure described for the synthesis of example 173, step 3, the title compound was prepared from (3,4-dimethoxy-phenyl)-[2-(4-trifluoromethyl-phenyl)-ethyl]-amine (example 173, step 2) and Boc-alpha-phenylglycine. MS (m/e): 559.4 [M+H]⁺.

### b) Step 2:

### 2-Amino-N-(3,4-dimethoxy-phenyl)-2-phenyl-N-[2-(4-trifluoromethyl-phenyl)-ethyl]-acetamide

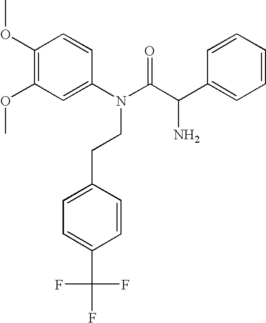

In analogy to the procedure described for the synthesis of example 173, step 4, the title compound was prepared from ({(3,4-dimethoxy-phenyl)-[2-(4-trifluoromethyl-phenyl)-ethyl]-carbamoyl}-phenyl-methyl)-carbamic acid tert-butyl ester. MS (m/e): 459.5 [M+H]⁺.

### c) Step 3:

### (R)-2-Amino-N-(3,4-dimethoxy-phenyl)-2-phenyl-N-[2-(4-trifluoromethyl-phenyl)-ethyl]-acetamide

The enantiomers of 2-Amino-N-(3,4-dimethoxy-phenyl)-2-phenyl-N-[2-(4-trifluoromethyl-phenyl)-ethyl]-acetamide (racemic mixture) were separated on a chiralpak AD column to provide the title compound as a light yellow oil (first eluting stereoisomer). MS (m/e): 459.3 [M+H]⁺.

### EXAMPLE 175

### (S)-2-Amino-N-(3,4-dimethyl-phenyl)-N-[2-(4-fluoro-phenyl)-ethyl]-2-phenyl-acetamide

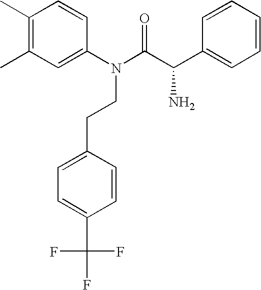

In analogy to the procedure described for the synthesis of example 173, steps: 1-4, the title compound was prepared from 3,4-dimethylaniline and 4-fluorophenyl acetic acid. MS (m/e): 377.4 [M+H]⁺.

### EXAMPLE 176

### (S)-2-Amino-N-(3,4-dimethyl-phenyl)-2-phenyl-N-[2-(4-trifluoromethoxy-phenyl)-ethyl]-acetamide

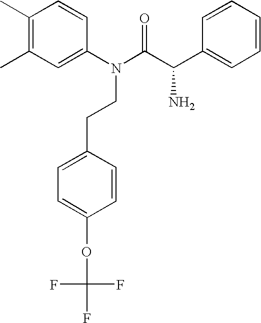

In analogy to the procedure described for the synthesis of example 173, steps: 1-4, the title compound was prepared from 3,4-dimethylaniline and (4-Trifluoromethoxy-phenyl)-acetic acid. MS (m/e): 377.4 [M+H]⁺.

### EXAMPLE 177

### (S)—N-(3,4-Dimethyl-phenyl)-N-[2-(4-fluoro-phenyl)-ethyl]-2-hydroxy-2-phenyl-acetamide

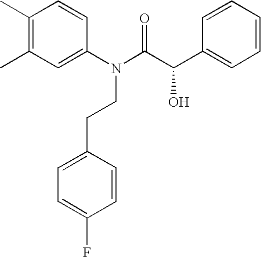

### a) Step 1:

### Acetic Acid (S)-{(3,4-dimethyl-phenyl)-[2-(4-fluoro-phenyl)-ethyl]-carbamoyl}-phenyl-methyl ester

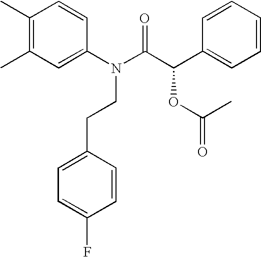

In analogy to the procedure described for the synthesis of example 173, steps: 3, the title compound was prepared from (3,4-dimethyl-phenyl)-[2-(4-fluoro-phenyl)-ethyl]-amine and (S)-(+)-O-acetyl-L-mandelic acid. MS (m/e): 420.2 [M+H]⁺.

### b) Step 2:

### (S)—N-(3,4-Dimethyl-phenyl)-N-[2-(4-fluoro-phenyl)-ethyl]-2-hydroxy-2-phenyl-acetamide

To a solution of 0.44 g (1.05 mmol) Acetic acid (S)-{(3,4-dimethyl-phenyl)-[2-(4-fluoro-phenyl)-ethyl]-carbamoyl}-phenyl-methyl ester in 4.4 ml tetrahydrofuran were added 2.2 ml water and 0.048 g (1.1 mmol) lithium hydroxide monohydrate. The mixture was stirred at room temperature for 70 hours then diluted with water and extracted 3 times with ethyl acetate. The combined extracts were dried over Na₂SO₄, filtered and concentrated in vacuo. The crude oil was purified with flash column chromatography on silica eluting with a gradient formed from heptane and ethylacetate to provide 0.31 g (78%) of the title compound as a colorless oil. MS (m/e): 378.3 [M+H]⁺.

### EXAMPLE 178

### (R)—N-(3,4-Dimethyl-phenyl)-N-[2-(4-fluoro-phenyl)-ethyl]-2-hydroxy-2-phenyl-acetamide

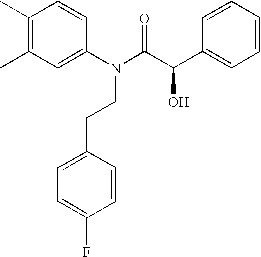

In analogy to the procedure described for the synthesis of example 177, steps: 1-2, the title compound was prepared from (3,4-dimethyl-phenyl)-[2-(4-fluoro-phenyl)-ethyl]-amine and (R)-(+)-O-acetyl-L-mandelic acid. MS (m/e): 378.3 [M+H]⁺.

### EXAMPLE 179

### (S)-2-Amino-N-[2-(2-chloro-phenyl)-ethyl]-N-(3,4-dimethyl-phenyl)-2-phenyl-acetamide hydrochloride

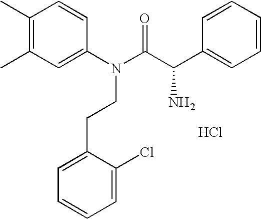

In analogy to the procedure described for the synthesis of example 173, steps: 1-4, the title compound was prepared from 3,4-dimethylaniline and (2-chloro-phenyl)-acetic acid. MS (m/e): 393.1 [M−HCl+H]⁺.

### EXAMPLE 180

### (S)-2-Amino-N-(3,4-dimethyl-phenyl)-N-[2-(2-fluoro-phenyl)-ethyl]-2-phenyl-acetamide hydrochloride

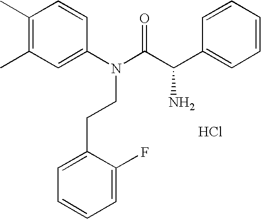

In analogy to the procedure described for the synthesis of example 173, steps: 1-4, the title compound was prepared from 3,4-dimethylaniline and (2-fluoro-phenyl)-acetic acid. MS (m/e): 377.4 [M+H]⁺.

### EXAMPLE 181

### (S)-2-Amino-N-(3,4-dimethyl-phenyl)-2-phenyl-N-(2-m-tolyl-ethyl)-acetamide hydrochloride

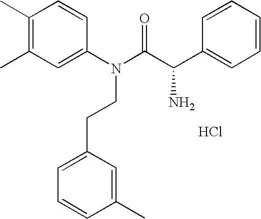

In analogy to the procedure described for the synthesis of example 173, steps: 1-4, the title compound was prepared from 3,4-dimethylaniline and (3-methyl-phenyl)-acetic acid. MS (m/e): 373.3 [M+H]⁺.

### EXAMPLE 182

### (S)-2-Amino-N-(3,4-dimethyl-phenyl)-N-[2-(3-fluoro-phenyl)-ethyl]-2-phenyl-acetamide hydrochloride

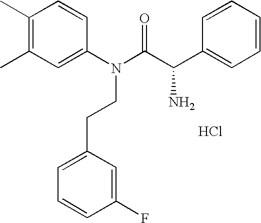

In analogy to the procedure described for the synthesis of example 173, steps: 1-4, the title compound was prepared from 3,4-dimethylaniline and (3-fluoro-phenyl)-acetic acid. MS (m/e): 377.4 [M+H]⁺.

### EXAMPLE 183

### (S)-2-Amino-N-[2-(3-chloro-phenyl)-ethyl]-N-(3,4-dimethyl-phenyl)-2-phenyl-acetamide hydrochloride

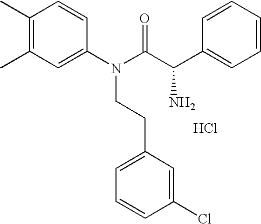

In analogy to the procedure described for the synthesis of example 173, steps: 1-4, the title compound was prepared from 3,4-dimethylaniline and (3-chloro-phenyl)-acetic acid. MS (m/e): 393.1 [M+H]⁺.

### EXAMPLE 184

### (S)-2-Amino-N-(3,4-dimethyl-phenyl)-2-phenyl-N-[2-(3-trifluoromethyl-phenyl)-ethyl]-acetamide hydrochloride

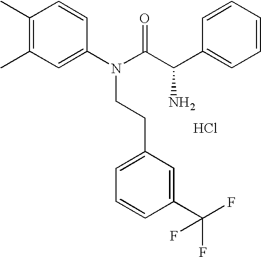

In analogy to the procedure described for the synthesis of example 173, steps: 1-4, the title compound was prepared from 3,4-dimethylaniline and (3-trifluoromethyl-phenyl)-acetic acid. MS (m/e): 427.4 [M+H]⁺.

### EXAMPLE 185

### (S)-2-Amino-N-(3,4-dimethyl-phenyl)-2-phenyl-N-(2-o-tolyl-ethyl)-acetamide hydrochloride

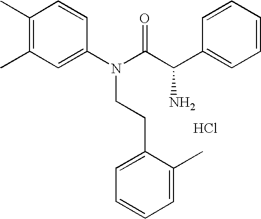

In analogy to the procedure described for the synthesis of example 173, steps: 1-4, the title compound was prepared from 3,4-dimethylaniline and (2-methyl-phenyl)-acetic acid. MS (m/e): 373.1 [M+H]⁺.

### EXAMPLE 186

### (S)-2-Amino-N-(3,4-dimethyl-phenyl)-N-[2-(3-fluoro-4-trifluoromethyl-phenyl)-ethyl]-2-phenyl-acetamide hydrochloride

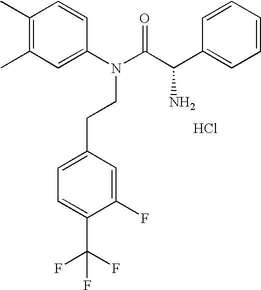

In analogy to the procedure described for the synthesis of example 173, steps: 1-4, the title compound was prepared from 3,4-dimethylaniline and (3-fluoro-4-trifluoromethyl-phenyl)-acetic acid. MS (m/e): 445.2 [M+H]⁺.

### EXAMPLE 187

### (S)-2-Amino-N-(3,4-dimethyl-phenyl)-N-[2-(4-fluoro-3-trifluoromethyl-phenyl)-ethyl]-2-phenyl-acetamide hydrochloride

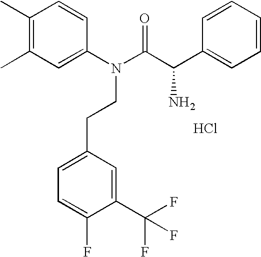

In analogy to the procedure described for the synthesis of example 173, steps: 1-4, the title compound was prepared from 3,4-dimethylaniline and (4-fluoro-3-trifluoromethyl-phenyl)-acetic acid. MS (m/e): 445.2 [M+H]⁺.

### EXAMPLE 188

### (S)-2-Amino-N-[2-(2,4-difluoro-phenyl)-ethyl]-N-(3,4-dimethyl-phenyl)-2-phenyl-acetamide hydrochloride

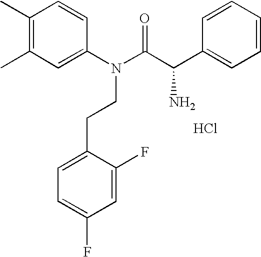

In analogy to the procedure described for the synthesis of example 173, steps: 1-4, the title compound was prepared from 3,4-dimethylaniline and (2,4-difluoro-phenyl)-acetic acid. MS (m/e): 395.2 [M+H]⁺.

### EXAMPLE 189

### (S)-2-Amino-N-[2-(3,4-difluoro-phenyl)-ethyl]-N-(3,4-dimethyl-phenyl)-2-phenyl-acetamide hydrochloride

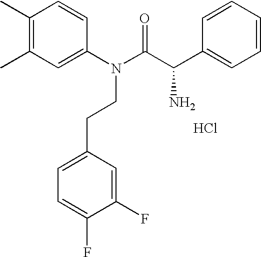

In analogy to the procedure described for the synthesis of example 173, steps: 1-4, the title compound was prepared from 3,4-dimethylaniline and (3,4-difluoro-phenyl)-acetic acid. MS (m/e): 395.2 [M+H]⁺.

### EXAMPLE 190

### (S)-2-Amino-N-(3,4-dimethyl-phenyl)-N-[2-(3-fluoro-4-methyl-phenyl)-ethyl]-2-phenyl-acetamide hydrochloride

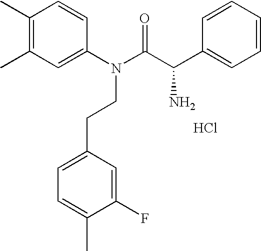

In analogy to the procedure described for the synthesis of example 173, steps: 1-4, the title compound was prepared from 3,4-dimethylaniline and (3-fluoro-4-methyl-phenyl)-acetic acid. MS (m/e): 391.3 [M+H]⁺.

### EXAMPLE 191

### (S)-2-Amino-N-[2-(2,3-difluoro-phenyl)-ethyl]-N-(3,4-dimethyl-phenyl)-2-phenyl-acetamide hydrochloride

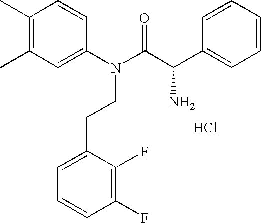

In analogy to the procedure described for the synthesis of example 173, steps: 1-4, the title compound was prepared from 3,4-dimethylaniline and (2,3-difluoro-phenyl)-acetic acid. MS (m/e): 395.2 [M+H]⁺.

### EXAMPLE 192

### (S)-2-Amino-N-[2-(4-chloro-2-fluoro-phenyl)-ethyl]-N-(3,4-dimethyl-phenyl)-2-phenyl-acetamide; hydrochloride

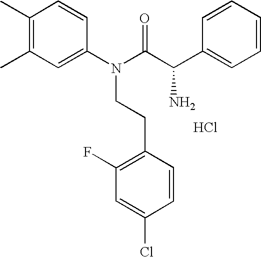

In analogy to the procedure described for the synthesis of example 173, steps: 1-4, the title compound was prepared from 3,4-dimethylaniline and (4-chloro-2-fluoro-phenyl)-acetic acid. MS (m/e): 411.2 [M+H]⁺.

### EXAMPLE 193

### (S)-2-Amino-N-(3,4-dimethyl-phenyl)-N-[2-(2-fluoro-5-trifluoromethyl-phenyl)-ethyl]-2-phenyl-acetamide; hydrochloride

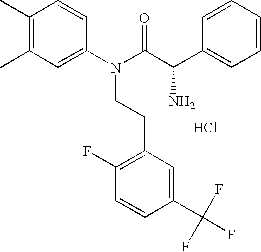

In analogy to the procedure described for the synthesis of example 173, steps: 1-4, the title compound was prepared from 3,4-dimethylaniline and (2-fluoro-5-trifluoromethyl-phenyl)-acetic acid. MS (m/e): 445.2 [M+H]⁺.

### EXAMPLE 194

### (S)-2-Amino-N-(3,4-dimethyl-phenyl)-N-[2-(2-methoxy-phenyl)-ethyl]-2-phenyl-acetamide hydrochloride

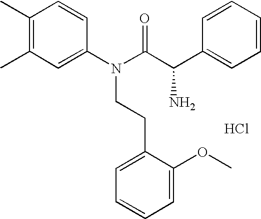

In analogy to the procedure described for the synthesis of example 173, steps: 1-4, the title compound was prepared from 3,4-dimethylaniline and (2-methoxy-phenyl)-acetic acid. MS (m/e): 389.1 [M+H]⁺.

### EXAMPLE 195

### (S)-2-Amino-N-(3,4-dimethyl-phenyl)-N-[2-(2-fluoro-3-trifluoromethyl-phenyl)-ethyl]-2-phenyl-acetamide hydrochloride

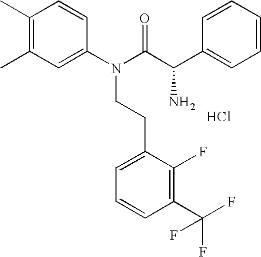

In analogy to the procedure described for the synthesis of example 173, steps: 1-4, the title compound was prepared from 3,4-dimethylaniline and (2-fluoro-3-trifluoromethyl-phenyl)-acetic acid. MS (m/e): 445.3 [M+H]⁺.

### EXAMPLE 196

### (S)-2-Amino-N-(3,4-dimethyl-phenyl)-N-[2-(2-fluoro-4-trifluoromethyl-phenyl)-ethyl]-2-phenyl-acetamide hydrochloride

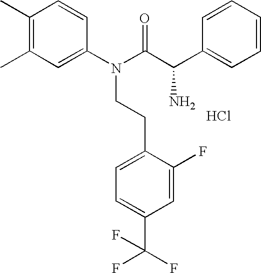

In analogy to the procedure described for the synthesis of example 173, steps: 1-4, the title compound was prepared from 3,4-dimethylaniline and (2-fluoro-4-trifluoromethyl-phenyl)-acetic acid. MS (m/e): 445.1 [M+H]⁺.

### EXAMPLE 197

### (S)-2-Amino-N-[2-(2,3-difluoro-4-trifluoromethyl-phenyl)-ethyl]-N-(3,4-dimethyl-phenyl)-2-phenyl-acetamide hydrochloride

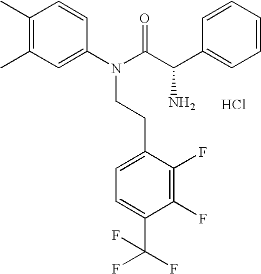

In analogy to the procedure described for the synthesis of example 173, steps: 1-4, the title compound was prepared from 3,4-dimethylaniline and (2,3-difluoro-4-trifluoromethyl-phenyl)-acetic acid. MS (m/e): 463.3 [M+H]⁺.

### EXAMPLE 198

### (S)-2-Amino-N-(3,4-dimethyl-phenyl)-2-phenyl-N-[2-(2-trifluoromethoxy-phenyl)-ethyl]-acetamide hydrochloride

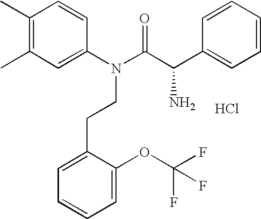

In analogy to the procedure described for the synthesis of example 173, steps: 1-4, the title compound was prepared from 3,4-dimethylaniline and (2-trifluoromethoxy-phenyl)-acetic acid. MS (m/e): 443.3 [M+H]⁺.

### EXAMPLE 199

### (S)-2-Amino-N-(3,4-dimethyl-phenyl)-N-phenethyl-2-phenyl-acetamide hydrochloride

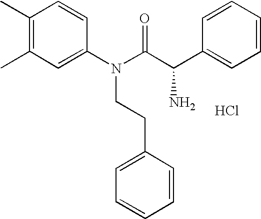

### a) Step 1:

### (3,4-Dimethyl-phenyl)-phenethyl-amine

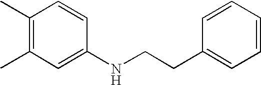

A dried flask was charged with 21 mg (0.11 mmol) CuI and 1.4 g (4.3 mmol) cesium carbonate under argon. 0.40 ml (3.2 mmol) phenethylamine, 0.5 g (2.1 mmol) 4-iodo-0-xylene in solution in 1 ml dried DMF and finally 0.058 ml (0.43 mmol) 2-acetylcyclohexanone were successively added. The mixture was stirred at room temperature for 24 hours. The mixture was diluted with water. The aqueous layer was extracted twice with ethyl acetate. The combined extracts were dried over Na₂SO₄, filtered and concentrated in vacuo. The crude oil was purified with flash column chromatography on silica eluting with a gradient formed from heptane and ethylacetate to provide 0.10 g (22%) of the title compound as a yellow oil. MS (m/e): 226.2 (MH⁺).

### b) Step 2:

(S)-2-Amino-N-(3,4-dimethyl-phenyl)-N-phenethyl-2-phenyl-acetamide hydrochloride

In analogy to the procedure described for the synthesis of example 173, steps: 3-4, the title compound was prepared from (3,4-dimethyl-phenyl)-phenethyl-amine. MS (m/e): 359.3 [M+H]⁺.

### EXAMPLE 200

### (S)-2-Amino-N-[2-(4-chloro-3-fluoro-phenyl)-ethyl]-N-(3,4-dimethyl-phenyl)-2-phenyl-acetamide hydrochloride

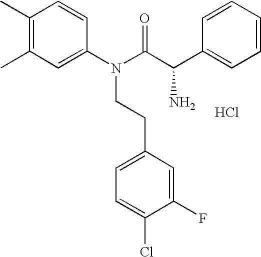

In analogy to the procedure described for the synthesis of example 173, steps: 1-4, the title compound was prepared from 3,4-dimethylaniline and (4-chloro-3-fluoro-phenyl)-acetic acid. MS (m/e): 411.2 [M+H]⁺.

### EXAMPLE 201

### (S)-2-Amino-N-(3,4-dimethyl-phenyl)-N-[2-(3-fluoro-5-trifluoromethyl-phenyl)-ethyl]-2-phenyl-acetamide hydrochloride

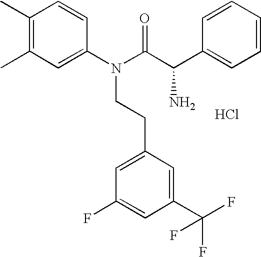

In analogy to the procedure described for the synthesis of example 173, steps: 1-4, the title compound was prepared from 3,4-dimethylaniline and (3-fluoro-5-trifluoromethyl-phenyl)-acetic acid. MS (m/e): 445.3 [M+H]⁺.

### EXAMPLE 202

### (S)-2-Amino-N-(3,4-dimethyl-phenyl)-2-phenyl-N-[2-(2,3,6-trifluoro-4-trifluoromethyl-phenyl)-ethyl]-acetamide hydrochloride

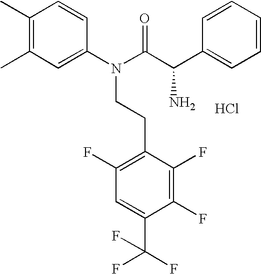

In analogy to the procedure described for the synthesis of example 173, steps: 1-4, the title compound was prepared from 3,4-dimethylaniline and (2,3,6-trifluoro-4-trifluoromethyl-phenyl)-acetic acid. MS (m/e): 481.3 [M+H]⁺.

### EXAMPLE 203

### (S)-2-Amino-N-[2-(2,5-dichloro-phenyl)-ethyl]-N-(3,4-dimethyl-phenyl)-2-phenyl-acetamide hydrochloride

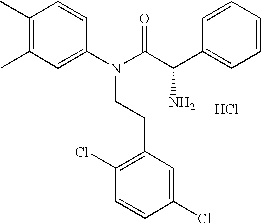

In analogy to the procedure described for the synthesis of example 199, steps: 1-2, the title compound was prepared from 4-iodo-O-xylene and 2-(2,5-dichloro-phenyl)-ethylamine. MS (m/e): 427.1 [M+H]⁺.

### EXAMPLE 204

### (S)-2-Amino-N-[2-(3-chloro-2-fluoro-phenyl)-ethyl]-N-(3,4-dimethyl-phenyl)-2-phenyl-acetamide hydrochloride

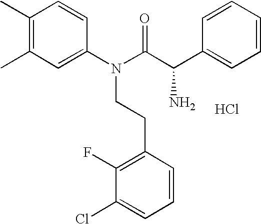

In analogy to the procedure described for the synthesis of example 199, steps: 1-2, the title compound was prepared from 4-iodo-O-xylene and 2-(3-chloro-2-fluoro-phenyl)-ethylamine. MS (m/e): 411.2 [M+H]⁺.

### EXAMPLE 205

### (S)-2-Amino-N-[2-(2,4-dichloro-phenyl)-ethyl]-N-(3,4-dimethyl-phenyl)-2-phenyl-acetamide hydrochloride

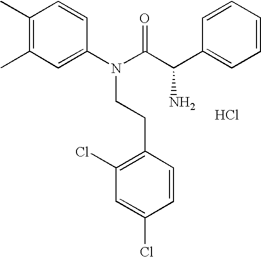

In analogy to the procedure described for the synthesis of example 199, steps: 1-2, the title compound was prepared from 4-iodo-O-xylene and 2-(2,4-dichloro-phenyl)-ethylamine. MS (m/e): 427.1 [M+H]⁺.

### EXAMPLE 206

### (S)-2-Amino-N-(3,4-dimethyl-phenyl)-N-[2-(3-hydroxy-phenyl)-ethyl]-2-phenyl-acetamide hydrochloride

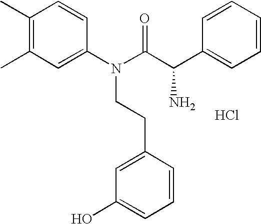

In analogy to the procedure described for the synthesis of example 199, steps: 1-2, the title compound was prepared from 4-iodo-O-xylene and 3-(2-amino-ethyl)-phenol. MS (m/e): 375.2 [M+H]⁺.

### EXAMPLE 207

### N-(3,4-Dimethyl-phenyl)-2-oxo-2-phenyl-N-[2-(2,3,6-trifluoro-4-trifluoromethyl-phenyl)-ethyl]-acetamide

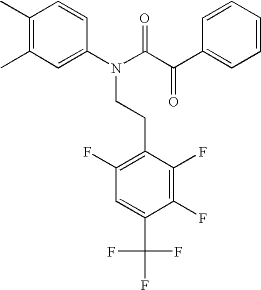

In analogy to the procedure described for the synthesis of example 173, step: 3, the title compound was prepared from (3,4-dimethyl-phenyl)-[2-(2,3,6-trifluoro-4-trifluoromethyl-phenyl)-ethyl]-amine (intermediate for example 202) and benzoylformic acid. MS (m/e): 480.1 [M+H]⁺.

### EXAMPLE 208

### (S)-2-Amino-N-[2-(3,5-difluoro-phenyl)-ethyl]-N-(3,4-dimethyl-phenyl)-2-phenyl-acetamide hydrochloride

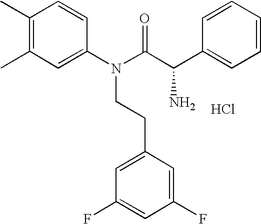

In analogy to the procedure described for the synthesis of example 199, steps: 1-2, the title compound was prepared from 4-iodo-O-xylene and 2-(3,5-difluoro-phenyl)-ethylamine. MS (m/e): 395.1 [M+H]⁺.

### EXAMPLE 209

### (S)-2-Amino-N-[2-(3-chloro-5-fluoro-phenyl)-ethyl]-N-(3,4-dimethyl-phenyl)-2-phenyl-acetamide hydrochloride

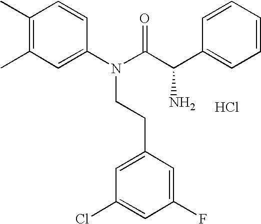

In analogy to the procedure described for the synthesis of example 199, steps: 1-2, the title compound was prepared from 4-iodo-0-xylene and 2-(3-chloro-5-fluoro-phenyl)-ethylamine. MS (m/e): 411.2 [M+H]⁺.

### EXAMPLE 210

### (S)-2-Amino-N-[2-(4-difluoromethoxy-phenyl)-ethyl]-N-(3,4-dimethyl-phenyl)-2-phenyl-acetamide hydrochloride

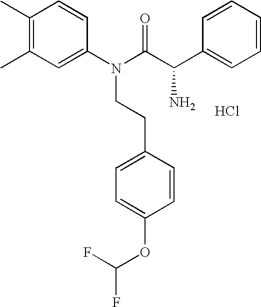

In analogy to the procedure described for the synthesis of example 199, steps: 1-2, the title compound was prepared from 4-iodo-O-xylene and 2-(4-difluoromethoxy-phenyl)-ethylamine. MS (m/e): 425.4 [M+H]⁺.

### EXAMPLE 211

### N-(3,4-Dimethyl-phenyl)-2-[hydroxyimino]-2-phenyl-N-[2-(2,3,6-trifluoro-4-trifluoromethyl-phenyl)-ethyl]-acetamide

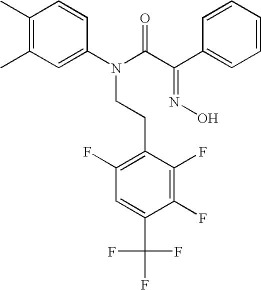

To a solution of 50 mg (0.1 mmol) N-(3,4-dimethyl-phenyl)-2-oxo-2-phenyl-N-[2-(2,3,6-trifluoro-4-trifluoromethyl-phenyl)-ethyl]-acetamide (example 207) in 2 ml ethanol at room temperature, was added 11.7 mg (0.17 mmol) hydroxylamine hydrochloride, followed by 27.14 mg (0.42 mmol) KOH. The reaction mixture was refluxed overnight then cooled to room temperature, water was added followed by HCl 1N. Dichloromethane was added and the water phase was extracted 3 times with dichloromethane. The combined extracts were dried over Na₂SO₄, filtered and concentrated in vacuo. The crude oil was purified with flash column chromatography on silica eluting with a gradient formed from heptane and ethylacetate to provide 10 mg (37%) of the title compound as a light red oil. MS (m/e): 495.2 [M+H]⁺.

### EXAMPLE 212

### (S)-2-Amino-N-[2-(4-cyano-phenyl)-ethyl]-N-(3,4-dimethyl-phenyl)-2-phenyl-acetamide hydrochloride

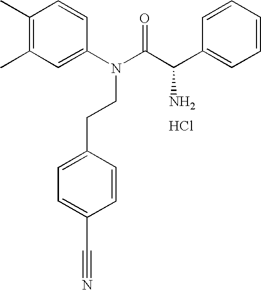

### a) Step 1:

### {(S)-[[2-(4-Bromo-phenyl)-ethyl]-(3,4-dimethyl-phenyl)-carbamoyl]-phenyl-methyl}-carbamic acid tert-butyl ester

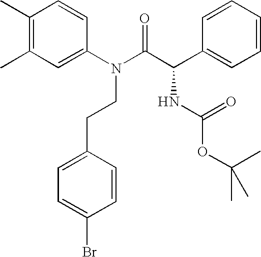

In analogy to the procedure described for the synthesis of example 173, steps: 1-3, the title compound was prepared from 3,4-dimethylaniline and (4-bromo-phenyl)-acetic acid. MS (m/e): 537.2 [M+H]⁺.

### b) Step 2:

### {(S)-[[2-(4-Cyano-phenyl)-ethyl]-(3,4-dimethyl-phenyl)-carbamoyl]-phenyl-methyl}-carbamic acid tert-butyl ester

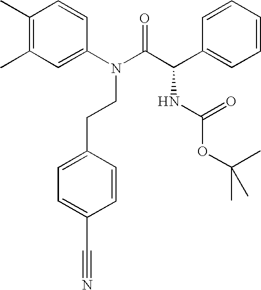

A solution of 0.16 g (0.3 mmol) {(S)-[[2-(4-Bromo-phenyl)-ethyl]-(3,4-dimethyl-phenyl)-carbamoyl]-phenyl-methyl}-carbamic acid tert-butyl ester, 0.11 g (1.2 mmol) copper cyanide, 0.016 g (0.015 mmol) Pd₂dba₃, 0.048 g (0.3 mmol) tetraethylammonium cyanide and 0.033 g (0.06 mmol) dppf in 2 ml dioxane was refluxed overnight at 105° C.

The solution was washed once with a saturated solution of NaHCO₃. The aqueous layer was extracted four times with ethylacetate. The combined extracts were dried over Na₂SO₄, filtered and concentrated in vacuo. The crude oil was purified with flash column chromatography on silica eluting with a gradient formed from heptane and ethylacetate to provide 79 mg (54%) of the title compound as a light yellow gum. MS (m/e): 484.4 [M+H]⁺.

### c) Step 3:

### (S)-2-Amino-N-[2-(4-cyano-phenyl)-ethyl]-N-(3,4-dimethyl-phenyl)-2-phenyl-acetamide hydrochloride

In analogy to the procedure described for the synthesis of example 173, step: 4, the title compound was prepared from {(S)-[[2-(4-cyano-phenyl)-ethyl]-(3,4-dimethyl-phenyl)-carbamoyl]-phenyl-methyl}-carbamic acid tert-butyl ester. MS (m/e): 384.1 [M+H]⁺.

### EXAMPLE 213

### 2,3-Dihydro-1H-isoindole-1-carboxylic acid (3,4-dimethyl-phenyl)-[2-(2,3,6-trifluoro-4-trifluoromethyl-phenyl)-ethyl]-amide hydrochloride (not encompassed by formula I)

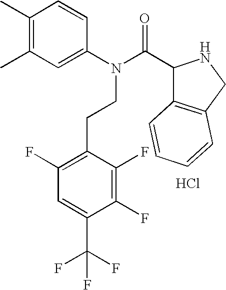

In analogy to the procedure described for the synthesis of example 173, steps: 3-4, the title compound was prepared from (3,4-dimethyl-phenyl)-[2-(2,3,6-trifluoro-4-trifluoromethyl-phenyl)-ethyl]-amine (intermediate for example 202) and Boc-1,3-dihydro-2H-isoindole carboxylic acid. MS (m/e): 493.3 [M+H]⁺.

### EXAMPLE 214

### 2-Amino-2-(5-chloro-thiophen-2-yl)-N-[2-(3,4-difluoro-phenyl)-ethyl]-N-(3,4-dimethyl-phenyl)-acetamide hydrochloride

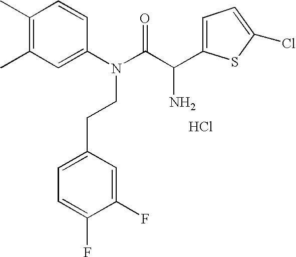

In analogy to the procedure described for the synthesis of example 173, steps: 3-4, the title compound was prepared from [2-(3,4-difluoro-phenyl)-ethyl]-(3,4-dimethyl-phenyl)-amine (intermediate for example 189) and tert-butoxycarbonylamino-(5-chloro-thiophen-2-yl)-acetic acid (CAS: 89379-87-3). MS (m/e): 435.4 [M+H]⁺.

### EXAMPLE 215

### 2-Amino-2-(6-chloro-pyridin-3-yl)-N-[2-(3,4-difluoro-phenyl)-ethyl]-N-(3,4-dimethyl-phenyl)-acetamide hydrochloride

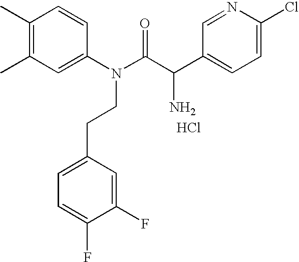

In analogy to the procedure described for the synthesis of example 173, steps: 3-4, the title compound was prepared from [2-(3,4-difluoro-phenyl)-ethyl]-(3,4-dimethyl-phenyl)-amine (intermediate for example 189) and tert-butoxycarbonylamino-(6-chloro-pyridin-3-yl)-acetic acid. MS (m/e): 430.4 [M+H]⁺.

### EXAMPLE 216

### 2-Amino-N-[2-(3,4-difluoro-phenyl)-ethyl]-N-(3,4-dimethyl-phenyl)-2-thiophen-3-yl-acetamide hydrochloride

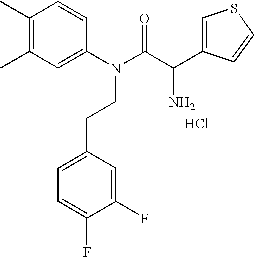

In analogy to the procedure described for the synthesis of example 173, steps: 3-4, the title compound was prepared from [2-(3,4-difluoro-phenyl)-ethyl]-(3,4-dimethyl-phenyl)-amine (intermediate for example 189) and tert-butoxycarbonylamino-thiophen-3-yl-acetic acid (CAS: 40512-57-0). MS (m/e): 401.2 [M+H]⁺.

### EXAMPLE 217

### 2-Amino-2-(4-cyano-phenyl)-N-[2-(3,4-difluoro-phenyl)-ethyl]-N-(3,4-dimethyl-phenyl)-acetamide hydrochloride

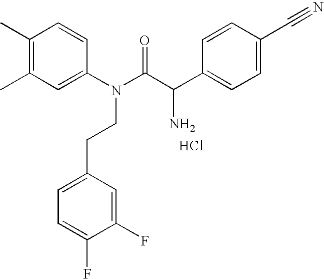

In analogy to the procedure described for the synthesis of example 173, steps: 3-4, the title compound was prepared from [2-(3,4-difluoro-phenyl)-ethyl]-(3,4-dimethyl-phenyl)-amine (intermediate for example 189) and tert-butoxycarbonylamino-(4-cyano-phenyl)-acetic acid. MS (m/e): 420.3 [M+H]⁺.

### EXAMPLE 218

### N-(3,4-Dimethyl-phenyl)-2-oxo-2-phenyl-N-[2-(4-trifluoromethyl-phenyl)-ethyl]-acetamide

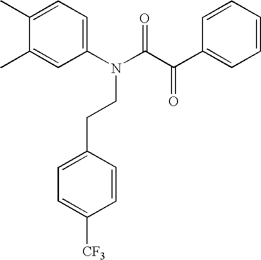

### a) Step 1:

### (3,4-dimethyl-phenyl)-[2-(4-trifluoromethyl-phenyl)-ethyl]-amine

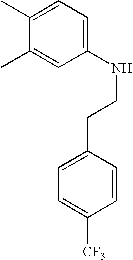

To a solution of 3,4-dimethylanilin (9.18 g, 76 mmol) and (4-trifluoromethyl)-phenylacetic acid (15.47 g, 76 mmol) in dichloromethane (500 mL) were added at 0° C. 1-(3-dimethylaminopropyl)-3-ethylcarbodiimide hydrochloride (15.98 g, 83 mmol), 1-hydroxybenzotriazole (1.02 g, 8 mmol) and N,N-diisopropyl ethyl amine (19.46 mL, 114 mmol). The resulting reaction mixture was stirred at this temperature for 20 min before allowing to warm to ambient temperature. After 2.5 h it was washed with aqueous sodium carbonate (half-saturated), aqueous HCl (1 M) and brine. Drying over sodium sulfate and concentration afforded an off-white solid which was dissolved in THF (300 mL) and borane-tetrahydrofuran complex (1 M in THF, 113.3 mL, 113 mmol) was added at ambient temperature over a period of 30 min. The reaction mixture was heated at 55° C. for 20 h before carefully addition of aqueous HCl (1 M, 100 mL) and additional heating at 80° C. for 30 min. After cooling to ambient temperature it was diluted with water (200 mL) basified with sodium carbonate. Extraction with diethylether was followed by washing with aqueous NaHCO3 (saturated) and brine. Drying over sodium sulfate afforded the title compound (16.2 g, 73%) as a yellow oil. MS m/e: 294.1 [M+H]⁺.

### b) Step 2:

### N-(3,4-Dimethyl-phenyl)-2-oxo-2-phenyl-N-[2-(4-trifluoromethyl-phenyl)-ethyl]-acetamide

To a solution of (3,4-dimethyl-phenyl)-[2-(4-trifluoromethyl-phenyl)-ethyl]-amine (5.00 g, 17.0 mmol) and benzoylformic acid (2.61 g, 17.0 mmol) in dichloromethane (110 mL) was added 1-(3-dimethylaminopropyl)-3-ethylcarbodiimide hydrochloride (3.67 g, 18.8 mmol), 1-hydroxybenzotriazole (235 mg, 1.70 mmol) and N-ethyldiisopropylamine (4.5 mL, 25.6 mmol) at 0° C. After stirring for 20 min at 0° C., the reaction mixture was allowed to reach ambient temperature and stirring was continued for 23 h. The mixture was washed with aqueous sodium carbonate (half-saturated, 100 mL), aqueous HCl (1 M, 100 mL) and brine (100 mL). The aqueous layers were washed with dichloromethane (100 mL). The combined organic layers were dried over sodium sulfate. Concentration and purification by chromatography (SiO₂, heptane:ethyl acetate=80:20) afforded the title compound (1.96 g, 27%) as a light-yellow oil. MS m/e: 426.1 [M+H]⁺.

### EXAMPLE 219

### (R,S)—N-(3,4-Dimethyl-phenyl)-2-hydroxy-2-phenyl-N-[2-(4-trifluoromethyl-phenyl)-ethyl]-propionamide

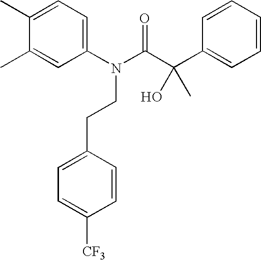

To a cooled solution of N-(3,4-dimethyl-phenyl)-2-oxo-2-phenyl-N-[2-(4-trifluoromethyl-phenyl)-ethyl]-acetamide (425 mg, 1.0 mmol) in diethylether (9.0 mL) was added dropwise methylmagnesium bromide solution (3 M in diethylether, 0.50 mL, 1.5 mmol) at 0° C. When the addition was completed the ice-bath was removed and the reaction mixture was stirred at ambient temperature for 1.5 h. It was quenched by the addition of aqueous NH4Cl (20%, 15 mL). The organic layer was separated and washed with brine (15 mL). The combined aqueous layers were extracted with diethylether (twice 15 mL). The combined organic layers were dried over sodium sulfate. Concentration and purification by chromatography (SiO₂, heptane:ethyl acetate=100:0 to 67:33) afforded the title compound (394 mg, 89%) as an off-white solid. MS m/e: 442.2 [M+H]⁺.

### EXAMPLE 220

### (R,S)—N-(3,4-Dimethyl-phenyl)-2-hydroxy-2-phenyl-N-[2-(4-trifluoromethyl-phenyl)-ethyl]-butyramide

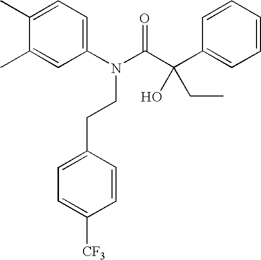

In analogy to the procedure described for the synthesis of example 219, the title compound (MS m/e: 456.3 [M+H]⁺) was prepared from N-(3,4-dimethyl-phenyl)-2-oxo-2-phenyl-N-[2-(4-trifluoromethyl-phenyl)-ethyl]-acetamide and ethylmagnesium bromide solution (3 M in diethylether).

### EXAMPLE 221

### (R,S)—N-(3,4-Dimethyl-phenyl)-2-ethoxy-2-phenyl-N-[2-(4-trifluoromethyl-phenyl)-ethyl]-acetamide

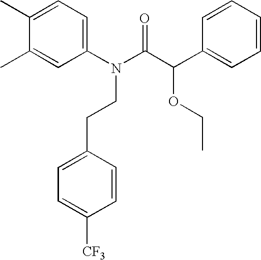

To a cooled solution of N-(3,4-dimethyl-phenyl)-2-oxo-2-phenyl-N-[2-(4-trifluoromethyl-phenyl)-ethyl]-acetamide (213 mg, 0.5 mmol) in diethylether (4.5 mL) was added dropwise ethylmagnesium bromide solution (3 M in diethylether, 0.25 mL, 0.75 mmol) at 0° C. When the addition was completed the ice-bath was removed and the reaction mixture was stirred at ambient temperature for 1.5 h. It was quenched by the addition of aqueous NH4Cl (20%, 15 mL). The organic layer was separated and washed with brine (15 mL). The combined aqueous layers were extracted with diethylether (twice 15 mL). The combined organic layers were dried over sodium sulfate. Concentration and purification by chromatography (SiO₂, heptane:ethyl acetate=100:0 to 67:33) afforded the title compound (87 mg, 38%) as a colorless oil. MS m/e: 456.3 [M+H]⁺.

### EXAMPLE 222

### (R,S)—N-(3,4-Dimethyl-phenyl)-2-isopropoxy-2-phenyl-N-[2-(4-trifluoromethyl-phenyl)-ethyl]-acetamide

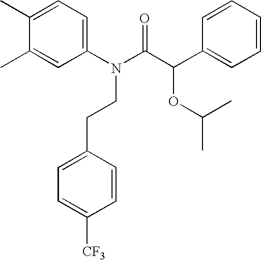

In analogy to the procedure described for the synthesis of example 221, the title compound (MS m/e: 470.2 [M+H]⁺) was prepared from N-(3,4-dimethyl-phenyl)-2-oxo-2-phenyl-N-[2-(4-trifluoromethyl-phenyl)-ethyl]-acetamide and isopropylmagnesium bromide solution (1 M in THF).

### EXAMPLE 223

### (R,S)—N-(3,4-Dimethyl-phenyl)-2-(4-fluoro-phenyl)-2-hydroxy-N-[2-(4-trifluoromethyl-phenyl)-ethyl]-propionamide

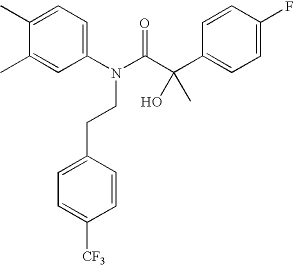

### a) Step 1:

### N-(3,4-Dimethyl-phenyl)-N-[2-(4-trifluoromethyl-phenyl)-ethyl]-oxalamic acid ethyl ester

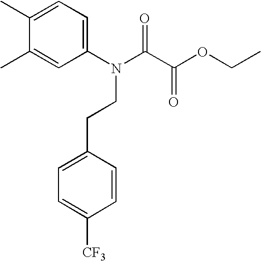

To a solution of (3,4-dimethyl-phenyl)-[2-(4-trifluoromethyl-phenyl)-ethyl]-amine (example 218, step 1, 3.00 g, 10.2 mmol) in diethylether (15 mL) was added triethylamine (1.57 mL, 11.2 mmol). The reaction mixture was cooled to 0° C. and ethyl oxalyl chloride (1.53 mL, 13.3 mmol) was added dropwise at 0° C. After stirring for 20 min at 0° C., the ice-bath was removed and it was stirred for 24 h at ambient temperature. It was washed with water (20 mL) and brine (15 mL). The aqueous layers were extracted with further diethylether (20 mL). The combined organic layers were dried over sodium sulfate. Concentration and purification by chromatography (SiO₂, heptane:ethyl acetate=100:0 to 67:33) afforded the title compound (3.89 g, 97%) as an off-white solid. MS m/e: 394.1 [M+H]⁺.

### b) Step 2:

### N-(3,4-Dimethyl-phenyl)-2-oxo-N-[2-(4-trifluoromethyl-phenyl)-ethyl]-propionamide

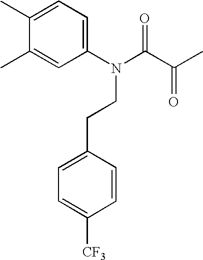

A solution of N-(3,4-dimethyl-phenyl)-N-[2-(4-trifluoromethyl-phenyl)-ethyl]-oxalamic acid ethyl ester (1.00 g, 2.54 mmol) in diethylether (120 mL) was cooled to −78° C. Then methylmagnesium bromide solution (3 M in diethylether, 1.02 mL, 3.06 mmol) was added dropwise and the reaction mixture was stirred for 3 h at −78° C. It was quenched with aqueous NH4Cl (saturated, 40 mL) at −78° C., allowed to warm to ambient temperature and extracted with diethylether (twice 100 mL). The combined organic layers were washed with brine (60 mL) and dried over sodium sulfate. Concentration and purification by chromatography (SiO₂, heptane:ethyl acetate=100:0 to 67:33) afforded the title compound (441 mg, 44%) as a colorless oil. MS m/e: 364.3 [M+H]⁺.

### c) Step 3:

### (R,S)—N-(3,4-Dimethyl-phenyl)-2-(4-fluoro-phenyl)-2-hydroxy-N-[2-(4-trifluoromethyl-phenyl)-ethyl]-propionamide

To a cooled solution of N-(3,4-dimethyl-phenyl)-2-oxo-N-[2-(4-trifluoromethyl-phenyl)-ethyl]-propionamide (182 mg, 0.5 mmol) in diethylether (4.5 mL) was added dropwise 4-fluorophenylmagnesium bromide solution (2 M in diethylether, 0.375 mL, 0.75 mmol) at 0° C. When the addition was completed the ice-bath was removed and the reaction mixture was stirred at ambient temperature for 1.5 h. It was quenched by addition of a aqueous NH4Cl (20%, 10 mL). The organic layer was separated and washed with brine (10 mL). The aqueous layers were extracted with diethylether (twice 10 mL). The combined organic layers were dried over sodium sulfate. Concentration and purification by chromatography (SiO₂, heptane:ethyl acetate=100:0 to 67:33) afforded the title compound (175 mg, 76%) as a colorless oil. MS m/e: 460.3 [M+H]⁺.

### EXAMPLE 224

### (S)—N-(3,4-Dimethyl-phenyl)-2-hydroxy-2-phenyl-N-[2-(4-trifluoromethyl-phenyl)-ethyl]-propionamide

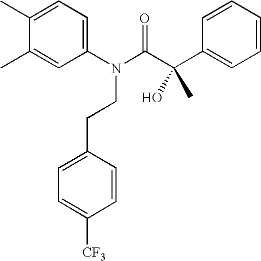

(R,S)—N-(3,4-Dimethyl-phenyl)-2-hydroxy-2-phenyl-N-[2-(4-trifluoromethyl-phenyl)-ethyl]-propionamide (example 219) was separated on chiral phase HPLC (Chiralpak AD column) to provide the title compound (S)—N-(3,4-dimethyl-phenyl)-2-hydroxy-2-phenyl-N-[2-(4-trifluoromethyl-phenyl)-ethyl]-propionamide (MS m/e: 424.2 [M+H]⁺) as a colorless oil.

### EXAMPLE 225

### (S)—N-(3,4-Dimethyl-phenyl)-2-ethoxy-2-phenyl-N-[2-(4-trifluoromethyl-phenyl)-ethyl]-acetamide

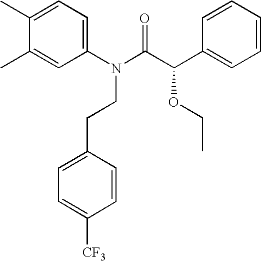

(R,S)—N-(3,4-Dimethyl-phenyl)-2-ethoxy-2-phenyl-N-[2-(4-trifluoromethyl-phenyl)-ethyl]-acetamide (example 221) was separated on chiral phase HPLC (Chiralpak AD column) to provide the title compound (S)—N-(3,4-dimethyl-phenyl)-2-ethoxy-2-phenyl-N-[2-(4-trifluoromethyl-phenyl)-ethyl]-acetamide (MS m/e: 456.2 [M+H]⁺) as a colorless oil.

### EXAMPLE 226

### (R)—N-(3,4-Dimethyl-phenyl)-2-ethoxy-2-phenyl-N-[2-(4-trifluoromethyl-phenyl)-ethyl]-acetamide

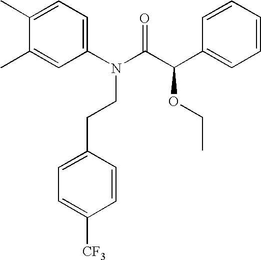

(R,S)—N-(3,4-Dimethyl-phenyl)-2-ethoxy-2-phenyl-N-[2-(4-trifluoromethyl-phenyl)-ethyl]-acetamide (example 221) was separated on chiral phase HPLC (Chiralpak AD column) to provide the title compound (R)—N-(3,4-dimethyl-phenyl)-2-ethoxy-2-phenyl-N-[2-(4-trifluoromethyl-phenyl)-ethyl]-acetamide (MS m/e: 456.2 [M+H]⁺) as a colorless oil.

### EXAMPLE 227

### (R,S)—N-(3,4-Dimethyl-phenyl)-2-hydroxy-2-pyridin-2-yl-N-[2-(4-trifluoromethyl-phenyl)-ethyl]-propionamide

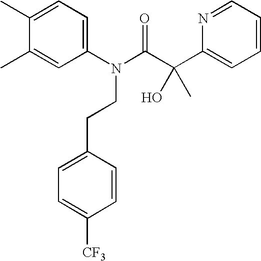

To a solution of 2-iodopyridine (129 mg, 0.60 mmol) in THF (1.5 mL) was added isopropylmagnesium chloride solution (2 M in THF, 275 μL, 0.55 mmol) at −40° C. After stirring for 45 min at −40° C., a solution of N-(3,4-dimethyl-phenyl)-2-oxo-N-[2-(4-trifluoromethyl-phenyl)-ethyl]-propionamide (example 223, step 2, 182 mg, 0.50 mmol) in THF (1.0 mL) was added and the reaction mixture was stirred for 25 h at −40° C. and then for 1 h at 0° C. The reaction mixture was allowed to rise to ambient temperature and was then quenched with brine (2.6 mL). After extraction with ethyl acetate (twice 20 mL), the organic layers were washed with brine (10 mL). The combined organic layers were dried over sodium sulfate. Concentration and purification by chromatography (SiO₂, heptane:ethyl acetate=95:5 to 50:50) afforded the title compound (92 mg, 42%) as a colorless oil. MS m/e: 443.2 [M+H]⁺.

### EXAMPLE 228

### (R,S)—N-(3,4-Dimethyl-phenyl)-2-hydroxy-2-pyridin-3-yl-N-[2-(4-trifluoromethyl-phenyl)-ethyl]-propionamide

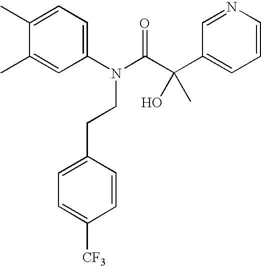

In analogy to the procedure described for the synthesis of example 227, the title compound (MS m/e: 443.2 [M+H]⁺) was prepared from 3-iodopyridine and N-(3,4-dimethyl-phenyl)-2-oxo-N-[2-(4-trifluoromethyl-phenyl)-ethyl]-propionamide (example 223, step 2).

### EXAMPLE 229

### 2-Benzoimidazol-1-yl-N-(3,4-dimethyl-phenyl)-N-[2-(4-trifluoromethyl-phenyl)-ethyl]-acetamide

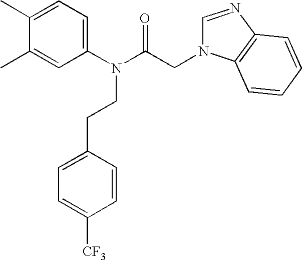

### a) Step 1:

### 2-Bromo-N-(3,4-dimethyl-phenyl)-N-[2-(4-trifluoromethyl-phenyl)-ethyl]-acetamide

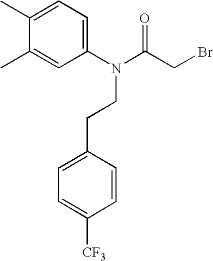

In analogy to the procedure described for the synthesis of example 223 (step 1), the title compound (MS m/e: 416.1 [M+H]⁺) was prepared from (3,4-dimethyl-phenyl)-[2-(4-trifluoromethyl-phenyl)-ethyl]-amine (example 218, step 1) and bromoacetyl chloride.

### b) Step 2:

### 2-Benzoimidazol-1-yl-N-(3,4-dimethyl-phenyl)-N-[2-(4-trifluoromethyl-phenyl)-ethyl]-acetamide

A mixture of 2-bromo-N-(3,4-dimethyl-phenyl)-N-[2-(4-trifluoromethyl-phenyl)-ethyl]-acetamide (104 mg, 0.25 mmol), benzimidazole (30 mg, 0.25 mmol) and potassium carbonate (69 mg, 0.5 mmol) in acetonitrile (2.5 mL) was stirred for 4 h at reflux. The reaction mixture was separated between ethyl acetate (twice 15 mL) and water (10 mL). The organic layers were washed with brine (10 mL), combined and dried over sodium sulfate. Concentration and purification by chromatography (SiO₂, heptane:ethyl acetate=75:25 to 25:75) afforded the title compound (75 mg, 66%) as a light yellow foam. MS m/e: 452.1 [M+H]⁺.

### EXAMPLE 230

### (R,S)-2-Benzoimidazol-1-yl-N-(3,4-dimethyl-phenyl)-N-[2-(4-trifluoromethyl-phenyl)-ethyl]-propionamide

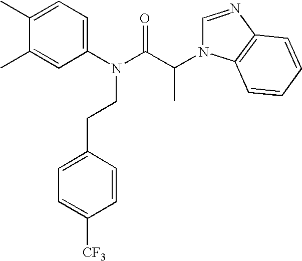

### a) Step 1:

### (R,S)-2-Bromo-N-(3,4-dimethyl-phenyl)-N-[2-(4-trifluoromethyl-phenyl)-ethyl]-propionamide

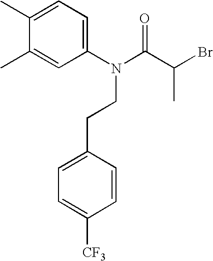

In analogy to the procedure described for the synthesis of example 223 (step 1), the title compound (MS m/e: 428.1/430.1 [M+H]⁺) was prepared from (3,4-dimethyl-phenyl)-[2-(4-trifluoromethyl-phenyl)-ethyl]-amine (example 218, step 1) and (R,S)-2-bromopropionyl chloride.

### b) Step 2:

### (R,S)-2-Benzoimidazol-1-yl-N-(3,4-dimethyl-phenyl)-N-[2-(4-trifluoromethyl-phenyl)-ethyl]-propionamide

In analogy to the procedure described for the synthesis of example 229 (step 2), the title compound (MS m/e: 466.3 [M+H]⁺) was prepared from (R,S)-2-bromo-N-(3,4-dimethyl-phenyl)-N-[2-(4-trifluoromethyl-phenyl)-ethyl]-propionamide and benzimidazole.

### EXAMPLE 231

### (R,S)—N-(3,4-Dimethyl-phenyl)-3-hydroxy-2-phenyl-N-[2-(4-trifluoromethyl-phenyl)-ethyl]-propionamide

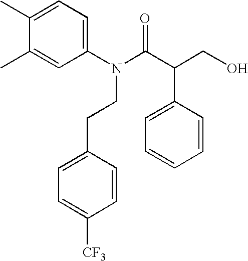

### a) Step 1:

### (R,S)—N-(3,4-Dimethyl-phenyl)-2-phenyl-N-[2-(4-trifluoromethyl-phenyl)-ethyl]-malonamic acid benzyl ester

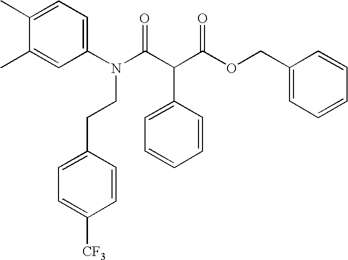

To a solution of phenylmalonic acid monobenzyl ester (1.08 g, 4.0 mmol) in dichloromethane (10 mL) at 0° C. was added dropwise 1-chloro-N,N,2-trimethylpropenylamine (635 uL, 4.80 mmol) and the resulting clear solution was stirred at this temperature for 2 h. After concentration, the residue was dissolved in THF (10 mL) and (3,4-dimethyl-phenyl)-[2-(4-trifluoromethyl-phenyl)-ethyl]-amine (example 218, step 1, 880 mg, 3.0 mmol) and ethyl N,N-diisopropyl amine (817 uL, 4.80 mmol) were added at 0° C. The resulting suspension was stirred at ambient temperature for 22 h. Aqueous sodium hydrogencarbonate (half-saturated) was added and the mixture extracted with ethyl acetate. The combined organic layers were dried over sodium sulfate and concentrated. Purification by chromatography (SiO₂, heptane:ethyl acetate 100:0 to 50:50) afforded the title compound (1.44 g, 66%) as a yellow oil. MS m/e: 546.3 [M+H]⁺.

### b) Step 2:

### (R,S)—N-(3,4-Dimethyl-phenyl)-3-hydroxy-2-phenyl-N-[2-(4-trifluoromethyl-phenyl)-ethyl]-propionamide

To a solution of (R,S)—N-(3,4-dimethyl-phenyl)-2-phenyl-N-[2-(4-trifluoromethyl-phenyl)-ethyl]-malonamic acid benzyl ester (1.36 g, 2.49 mmol) in methanol (8 mL) at 0° C. were added calcium chloride (277 mg, 2.49 mmol) and sodium borohydride (188 mg, 4.98 mmol) and the resulting light yellow suspension was stirred for 1.5 h at this temperature. After warming to ambient temperature stirring was continued for 6 h. After addition of aqueous HCl (1 N, 10 mL) it was extracted with ethyl acetate, washed with aqueous Na2CO3 (sat.) and dried over sodium sulfate. Concentration and purification by chromatography (SiO₂, heptane:ethyl acetate=100:0 to 50:50) afforded the title compound (860 g, 78%) as a yellow oil. MS m/e: 442.3 [M+H]⁺.

### EXAMPLE 232

### N-(3,4-Dimethyl-phenyl)-2-indazol-1-yl-N-[2-(4-trifluoromethyl-phenyl)-ethyl]-acetamide

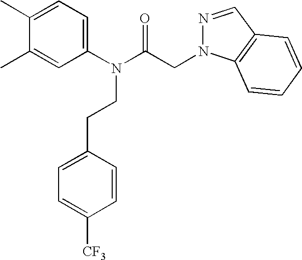

To a solution of 2-bromo-N-(3,4-dimethyl-phenyl)-N-[2-(4-trifluoromethyl-phenyl)-ethyl]-acetamide (211 mg, 0.68 mmol) in dichloromethane (2 mL) was added at 0° C. 1H-indazol-1-ylacetic acid (133 mg, 0.75 mmol) and 1-(3-dimethylaminopropyl)-3-ethylcarbodiimide hydrochloride (145 mg, 0.75 mmol). After stirring for 3 h at 0° C. it was allowed to warm to ambient temperature and stirring was continued for 66 h. Dichloromethane (4 mL) was added and the reaction mixture was washed with aqueous NaHCO₃ (3 mL) and water (4 mL). The aqueous layers were extracted with dichloromethane (4 mL). The combined organic layers were dried over sodium sulfate and concentrated. Purification by chromatography (SiO₂, heptane:ethyl acetate=100:0 to 60:40) afforded the title compound (193 mg, 59%) as a light-yellow oil. MS m/e: 452.1 [M+H]⁺.

### EXAMPLE 233

### N-(3,4-Dimethyl-phenyl)-2-imidazo[4,5-b]pyridin-1-yl-N-[2-(4-trifluoromethyl-phenyl)-ethyl]-acetamide

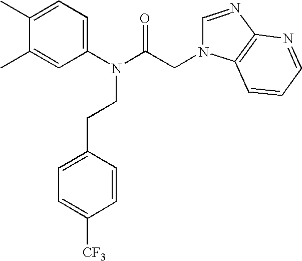

In analogy to the procedure described for the synthesis of example 229 (step 2), the title compound (MS m/e: 453.1 [M+H]⁺) was prepared from 2-bromo-N-(3,4-dimethyl-phenyl)-N-[2-(4-trifluoromethyl-phenyl)-ethyl]-acetamide and 4-azabenzimidazole.

### EXAMPLE 234

### N-(3,4-Dimethyl-phenyl)-2-purin-9-yl-N-[2-(4-trifluoromethyl-phenyl)-ethyl]-acetamide

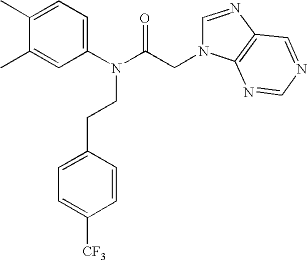

In analogy to the procedure described for the synthesis of example 229 (step 2), the title compound (MS m/e: 454.1 [M+H]⁺) was prepared from 2-bromo-N-(3,4-dimethyl-phenyl)-N-[2-(4-trifluoromethyl-phenyl)-ethyl]-acetamide and purine.

### EXAMPLE 235

### N-(3,4-Dimethyl-phenyl)-2-purin-7-yl-N-[2-(4-trifluoromethyl-phenyl)-ethyl]-acetamide

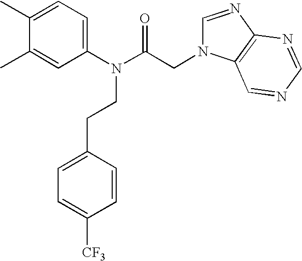

In analogy to the procedure described for the synthesis of example 229 (step 2), the title compound (MS m/e: 454.1 [M+H]⁺) was prepared from 2-bromo-N-(3,4-dimethyl-phenyl)-N-[2-(4-trifluoromethyl-phenyl)-ethyl]-acetamide and purine.

### EXAMPLE 236

### N-(3,4-Dimethyl-phenyl)-2-(2-methyl-benzoimidazol-1-yl)-N-[2-(4-trifluoromethyl-phenyl)-ethyl]-acetamide

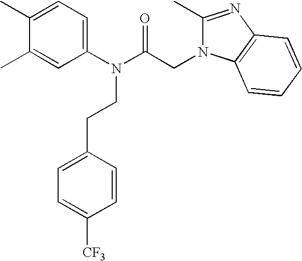

In analogy to the procedure described for the synthesis of example 229 (step 2), the title compound (MS m/e: 466.2[M+H]⁺) was prepared from 2-bromo-N-(3,4-dimethyl-phenyl)-N-[2-(4-trifluoromethyl-phenyl)-ethyl]-acetamide and 2-methylbenzimidazole.

### EXAMPLE 237

### (R,S)-2-Amino-N-(3,4-dimethyl-phenyl)-2-pyridin-3-yl-N-[2-(4-trifluoromethyl-phenyl)-ethyl]-acetamide hydrochloride

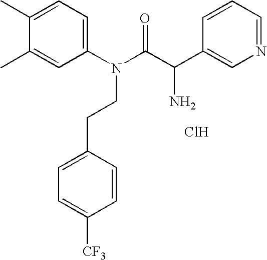

### a) Step 1:

### (R,S)-tert-Butoxycarbonylamino-pyridin-3-yl-acetic acid

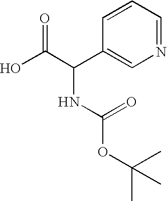

To a mixture of (R,S)-3-pyridinyl-aminoacetic acid hydrochloride (1.26 g, 6.70 mmol) and di-tert-butyl-dicarbonate (1.61 g, 7.40 mmol) were added THF (15 mL) and aqueous NaHCO₃ (1 M, 15 mL) and the resulting reaction mixture was stirred for 4 d at ambient temperature. Aqueous citric acid (5%, 25 mL) was added and extracted with ethyl acetate. The aqueous layer was extracted with ethyl acetate and the combined organic layers were washed with brine and dried over sodium sulfate. Concentration afforded the title compound (0.57 g, 34%) as a light-yellow solid which was directly used in the next step without further purification.

### b) Step 2:

### (R,S)-({(3,4-Dimethyl-phenyl)-[2-(4-trifluoromethyl-phenyl)-ethyl]-carbamoyl}-pyridin-3-yl-methyl)-carbamic Acid Tert-butyl Ester

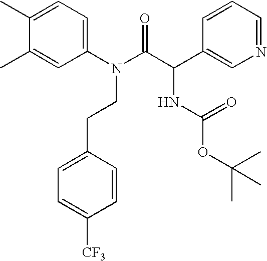

In analogy to the procedure described for the synthesis of example 232, the title compound (MS m/e: 528.0 [M+H]⁺) was prepared from (3,4-dimethyl-phenyl)-[2-(4-trifluoromethyl-phenyl)-ethyl]-amine and (R,S)-tert-butoxycarbonylamino-pyridin-3-yl-acetic acid.

### c) Step 3:

### (R,S)-2-Amino-N-(3,4-dimethyl-phenyl)-2-pyridin-3-yl-N-[2-(4-trifluoromethyl-phenyl)-ethyl]-acetamide hydrochloride

A reaction mixture of (R,S)-({(3,4-dimethyl-phenyl)-[2-(4-trifluoromethyl-phenyl)-ethyl]-carbamoyl}-pyridin-3-yl-methyl)-carbamic acid tert-butyl ester (150 mg, 0.32 mmol) and HCl (4 M in dioxane, 969 μL, 3.87 mmol) was stirred at ambient temperature for 48 h. The resulting suspension was diluted with heptane and filtered. Drying in vacuo afforded the title compound (116 mg, 66%) as a white solid. MS m/e: 428.2 [M+H]⁺.

### EXAMPLE 238

### (R,S)-2-Amino-N-(3,4-dimethyl-phenyl)-2-thiophen-2-yl-N-[2-(4-trifluoromethyl-phenyl)-ethyl]-acetamide

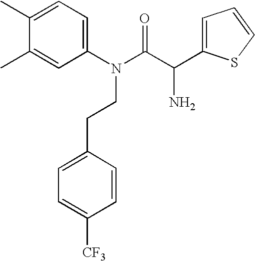

### a) Step 1:

### (R,S)-tert-Butoxycarbonylamino-thiophen-2-yl-acetic acid

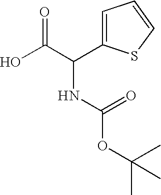

In analogy to the procedure described for the synthesis of example 237 (step 1), the title compound was prepared from DL-alpha-(2-thienyl)glycine and di-tert-butyl-dicarbonate.

### b) Step 2:

### (R,S)-({(3,4-Dimethyl-phenyl)-[2-(4-trifluoromethyl-phenyl)-ethyl]-carbamoyl}-thiophen-2-yl-methyl)-carbamic acid tert-butyl ester

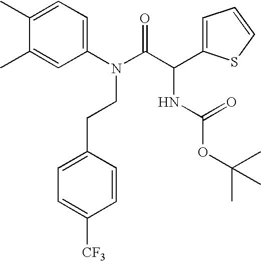

In analogy to the procedure described for the synthesis of example 232, the title compound (MS m/e: 533.2 [M+H]⁺) was prepared from (3,4-dimethyl-phenyl)-[2-(4-trifluoromethyl-phenyl)-ethyl]-amine and (R,S)-tert-butoxycarbonylamino-thiophen-2-yl-acetic acid.

### c) Step 3:

### (R,S)-2-Amino-N-(3,4-dimethyl-phenyl)-2-thiophen-2-yl-N-[2-(4-trifluoromethyl-phenyl)-ethyl]-acetamide hydrochloride

A reaction mixture of (R,S)-({(3,4-dimethyl-phenyl)-[2-(4-trifluoromethyl-phenyl)-ethyl]-carbamoyl}-thiophen-2-yl-methyl)-carbamic acid tert-butyl ester (150 mg, 0.32 mmol) and HCl (4 M in dioxane, 969 μL, 3.87 mmol) was stirred at ambient temperature for 48 h. It was diluted with TBME (2 mL) and washed with aqueous Na2CO3 (4 mL). The organic layer was dried over sodium sulfate. Concentration and purification by chromatography (SiO₂, heptane:ethyl acetate=33:67 to dichloromethane:methanol: ammonia=95:4.5:0.5) afforded the title compound (107 mg, 97%) as a yellow oil. MS m/e: 433.2 [M#H]⁺.

### EXAMPLE 239

### (S)-2-Amino-N-(2,3-dihydro-benzofuran-5-yl)-N-[2-(4-fluoro-phenyl)-ethyl]-2-phenyl-acetamide hydrochloride

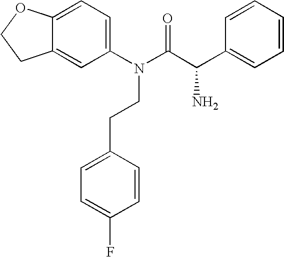

((S)-{(2,3-dihydro-benzofuran-5-yl)-[2-(4-fluoro-phenyl)-ethyl]-carbamoyl}-phenyl-methyl)-carbamic acid tert-butyl ester (example 240, 920 mg, 1.88 mmol) was dissolved in hydrochloric acid (4 M in dioxane, 5.6 mL, 23 mmol) and stirred for 4 h at 40° C. The solution was concentrated in vacuo and dissolved in TBME (5 mL). After the addition of heptane the mixture was stirred for 18 h at ambient temperature. The resulting suspension was filtered and washed with heptane. Drying afforded the title compound (556 mg, 69%) as a white solid. MS m/e: 391.1 [M+H]⁺.

### EXAMPLE 240

### ((S)-{(2,3-Dihydro-benzofuran-5-yl)-[2-(4-fluoro-phenyl)-ethyl]-carbamoyl}-phenyl-methyl)-carbamic Acid Tert-butyl Ester

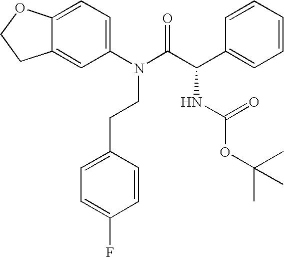

### a) Step 1:

### (2,3-Dihydro-benzofuran-5-yl)-[2-(4-fluoro-phenyl)-ethyl]-amine

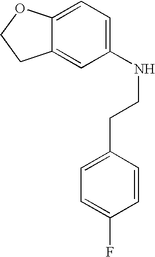

A mixture of 4-fluorophenethylamine (700 mg, 5.03 mmol), 5-bromo-2,3-dihydro-benzofuran (1.00 g, 5.03 mmol), copper(I) iodide (48 mg, 0.25 mmol) and cesium carbonate (3.28 g, 10.1 mmol), DMF (1.4 mL) and 2-acetylcyclohexanone (133 μl, 1.01 mmol) was stirred for 1 h at 120° C. under a argon atmosphere. After cooling to ambient temperature it was diluted with TBME (15 mL) and washed twice with water (10 mL) and brine (10 mL). The aqueous layers were extracted with TBME (15 mL). The combined organic layers were dried over sodium sulfate and concentrated. Purification by chromatography (SiO₂, heptane:ethyl acetate=100:0 to 70:30) afforded the title compound (860 mg, 66%) as a light-brown oil. MS m/e: 258.1 [M+H]⁺.

### b) Step 2:

### ((S)-{(2,3-Dihydro-benzofuran-5-yl)-[2-(4-fluoro-phenyl)-ethyl]-carbamoyl}-phenyl-methyl)-carbamic acid tert-butyl ester

In analogy to the procedure described for the synthesis of example 232, the title compound (MS m/e: 491.3 [M+H]⁺) was prepared from ((2,3-dihydro-benzofuran-5-yl)-[2-(4-fluoro-phenyl)-ethyl]-amine and N-alpha-BOC-L-phenylglycine.

### EXAMPLE 241

### 2-(1H-Benzoimidazol-2-yl)-N-(3,4-dimethyl-phenyl)-N-[2-(4-fluoro-phenyl)-ethyl]-acetamide

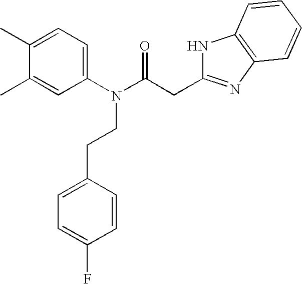

### a) Step 1:

### (3,4-Dimethyl-phenyl)-[2-(4-fluoro-phenyl)-ethyl]-amine

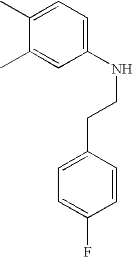

In analogy to the procedure described for the synthesis of example 240 (step 1), the title compound (MS m/e: 244.2 [M+H]⁺) was prepared from 4-fluorophenethylamine and 4-iodo-o-xylene.

### b) Step 2:

### 2-(1H-Benzoimidazol-2-yl)-N-(3,4-dimethyl-phenyl)-N-[2-(4-fluoro-phenyl)-ethyl]-acetamide

In analogy to the procedure described for the synthesis of example 232, the title compound (MS m/e: 402.4 [M+H]⁺) was prepared from (3,4-dimethyl-phenyl)-[2-(4-fluoro-phenyl)-ethyl]-amine and (1H-benzoimidazol-2-yl)-acetic acid.

### EXAMPLE 242

### 2-Methyl-2H-indazole-3-carboxylic acid (3,4-dimethyl-phenyl)-[2-(4-fluoro-phenyl)-ethyl]-amide (not encompassed by formula I)

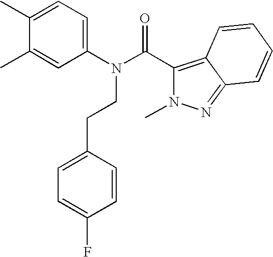

In analogy to the procedure described for the synthesis of example 232, the title compound (MS m/e: 402.4 [M+H]⁺) was prepared from (3,4-dimethyl-phenyl)-[2-(4-fluoro-phenyl)-ethyl]-amine and 2-methyl-2H-indazole-3-carboxylic acid.

### EXAMPLE 243

### (S)-2-Amino-N-(2,3-dihydro-benzofuran-6-yl)-N-[2-(4-fluoro-phenyl)-ethyl]-2-phenyl-acetamide hydrochloride

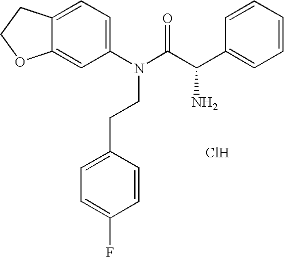

### a) Step 1:

### (2,3-Dihydro-benzofuran-6-yl)-[2-(4-fluoro-phenyl)-ethyl]-amine

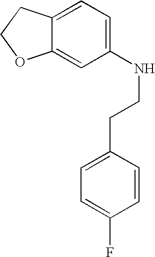

In analogy to the procedure described for the synthesis of example 240 (step 1), the title compound (MS m/e: 258.1 [M+H]⁺) was prepared from 4-fluorophenethylamine and 6-bromo-2,3-dihydro-benzofuran.

### b) Step 2:

### ((S)-{(2,3-Dihydro-benzofuran-6-yl)-[2-(4-fluoro-phenyl)-ethyl]-carbamoyl}-phenyl-methyl)-carbamic acid tert-butyl ester

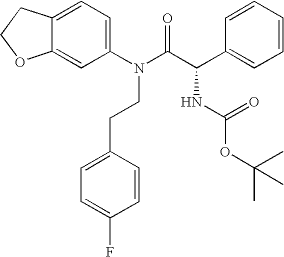

In analogy to the procedure described for the synthesis of example 232, the title compound (MS m/e: 491.3 [M+H]⁺) was prepared from ((2,3-dihydro-benzofuran-6-yl)-[2-(4-fluoro-phenyl)-ethyl]-amine and N-alpha-BOC-L-phenylglycine.

### c) Step 3:

### (S)-2-Amino-N-(2,3-dihydro-benzofuran-6-yl)-N-[2-(4-fluoro-phenyl)-ethyl]-2-phenyl-acetamide hydrochloride

((S)-{(2,3-dihydro-benzofuran-6-yl)-[2-(4-fluoro-phenyl)-ethyl]-carbamoyl}-phenyl-methyl)-carbamic acid tert-butyl ester (example 240, 135 mg, 0.275 mmol) was dissolved in hydrochloric acid (4 M in dioxane, 825 μL, 3.3 mmol) and stirred for 18 h at ambient temperature. The solution was concentrated in vacuo and dissolved in TBME (5 mL). After the addition of heptane the mixture was stirred for 18 h at ambient temperature. The upper layer was separated and the remaining residue was dried affording the title compound (112 mg, 95%) as light-brown foam. MS m/e: 374.2 [M+H]⁺.

### EXAMPLE 244

### (S)-2-Amino-N-(4-ethyl-phenyl)-N-[2-(4-fluoro-phenyl)-ethyl]-2-phenyl-acetamide hydrochloride

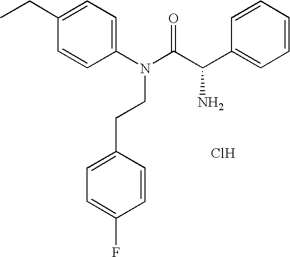

### a) Step 1:

### (4-Ethyl-phenyl)-[2-(4-fluoro-phenyl)-ethyl]-amine

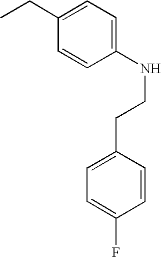

In analogy to the procedure described for the synthesis of example 240 (step 1), the title compound (MS m/e: 244.2 [M+H]⁺) was prepared from 4-fluorophenethylamine and 1-ethyl-4-iodobenzene.

### b) Step 2:

### ((S)-{(4-Ethyl-phenyl)-[2-(4-fluoro-phenyl)-ethyl]-carbamoyl}-phenyl-methyl)-carbamic acid tert-butyl ester

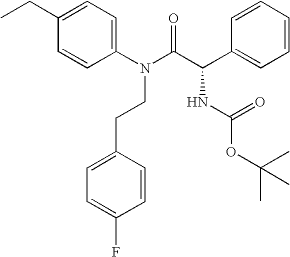

In analogy to the procedure described for the synthesis of example 232, the title compound (MS m/e: 477.2 [M+H]⁺) was prepared from (4-ethyl-phenyl)-[2-(4-fluoro-phenyl)-ethyl]-amine and N-alpha-BOC-L-phenylglycine.

### c) Step 3:

### (S)-2-Amino-N-(4-ethyl-phenyl)-N-[2-(4-fluoro-phenyl)-ethyl]-2-phenyl-acetamide hydrochloride

In analogy to the procedure described for the synthesis of example 239, the title compound (MS m/e: 377.3 [M+H]⁺) was prepared from ((S)-{(4-ethyl-phenyl)-[2-(4-fluoro-phenyl)-ethyl]-carbamoyl}-phenyl-methyl)-carbamic acid tert-butyl ester.

### EXAMPLE 245

### N-(3,4-Dimethyl-phenyl)-N-[2-(4-fluoro-phenyl)-ethyl]-2-(2-trifluoromethyl-benzoimidazol-1-yl)-acetamide

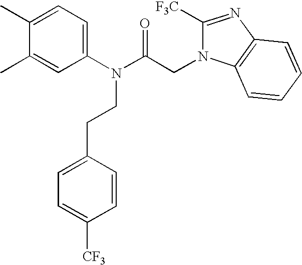

In analogy to the procedure described for the synthesis of example 229 (step 2), the title compound (MS m/e: 470.3 [M+H]⁺) was prepared from 2-bromo-N-(3,4-dimethyl-phenyl)-N-[2-(4-trifluoromethyl-phenyl)-ethyl]-acetamide and 2-trifluoromethyl-benzimidazole.

### EXAMPLE 246

### N-(2,3-Dihydro-benzofuran-6-yl)-N-[2-(4-fluoro-phenyl)-ethyl]-2-(2-methyl-benzoimidazol-1-yl)-acetamide

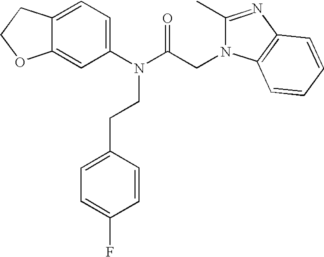

### a) Step 1:

### 2-Bromo-N-(2,3-dihydro-benzofuran-6-yl)-N-[2-(4-fluoro-phenyl)-ethyl]-acetamide

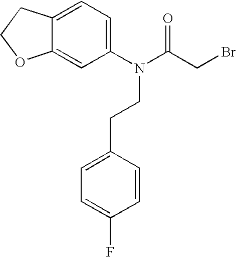

In analogy to the procedure described for the synthesis of example 223 (step 1), the title compound (MS m/e: 378.2 [M+H]⁺) was prepared from (2,3-dihydro-benzofuran-6-yl)-[2-(4-fluoro-phenyl)-ethyl]-amine (example 243, step 1) and bromoacetyl chloride.

### b) Step 2:

### N-(2,3-Dihydro-benzofuran-6-yl)-N-[2-(4-fluoro-phenyl)-ethyl]-2-(2-methyl-benzoimidazol-1-yl)-acetamide

In analogy to the procedure described for the synthesis of example 229 (step 2), the title compound (MS m/e: 430.4 [M+H]⁺) was prepared from 2-bromo-N-(2,3-dihydro-benzofuran-6-yl)-N-[2-(4-fluoro-phenyl)-ethyl]-acetamide and 2-methylbenzimidazole.

### EXAMPLE 247

### (R)-2-(2-Amino-acetylamino)-N-(3,4-dimethyl-phenyl)-2-phenyl-N-(2-p-tolyl-ethyl)-acetamide

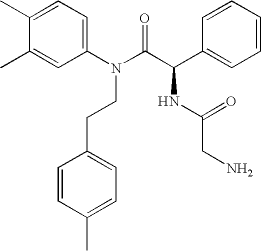

In analogy to the procedure described for the synthesis of example 53 (step 3), the title compound (−ve rotation) was prepared from ([({[(3,4-Dimethyl-phenyl)-(2-p-tolyl-ethyl)-carbamoyl]-phenyl-methyl}-carbamoyl)-methyl]-carbamic acid tert-butyl ester (prepared in example 168). MS (m/e): 430.2 [M+H]⁺.

### EXAMPLE 248

### (S)-2-(2-Amino-acetylamino)-N-(3,4-dimethyl-phenyl)-2-phenyl-N-(2-p-tolyl-ethyl)-acetamide

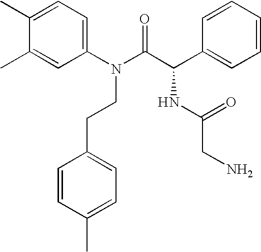

In analogy to the procedure described for the synthesis of example 53 (step 3), the title compound (+ve rotation) was prepared from ([({[(3,4-Dimethyl-phenyl)-(2-p-tolyl-ethyl)-carbamoyl]-phenyl-methyl}-carbamoyl)-methyl]-carbamic acid tert-butyl ester (prepared in example 168). MS (m/e): 430.2 [M+H]⁺.

### EXAMPLE 249

### (S)-2-((S)-2-Amino-2-phenyl-acetylamino)-N-(3,4-dimethyl-phenyl)-2-phenyl-N-(2-p-tolyl-ethyl)-acetamide

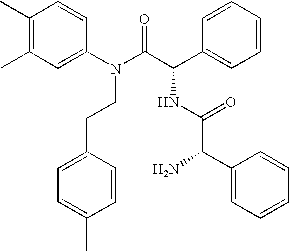

### a) Step 1:

### [(S)-({[(3,4-Dimethyl-phenyl)-(2-p-tolyl-ethyl)-carbamoyl]-phenyl-meth yl}-carbamoyl)-phenyl-methyl]-carbamic acid tert-butyl ester

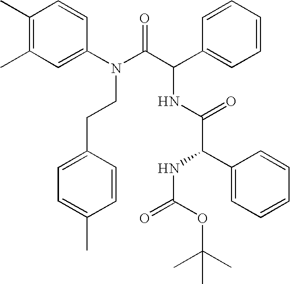

In analogy to the procedure described for the synthesis of example 168, the title compound was prepared from 2-amino-N-(3,4-dimethyl-phenyl)-2-phenyl-N-(2-p-tolyl-ethyl)-acetamide (from example 89, step 2) and t-Boc-phenylalanine to afford the title compound. MS (m/e): 606.2 [M+H]⁺.

### b) Step 2:

### (S)-2-((S)-2-Amino-2-phenyl-acetylamino)-N-(3,4-dimethyl-phenyl)-2-phenyl-N-(2-p-tolyl-ethyl)-acetamide

In analogy to the procedure described for the synthesis of example 53 (step 3), the title compound (+ve rotation) was prepared from [(S)-({[(3,4-Dimethyl-phenyl)-(2-p-tolyl-ethyl)-carbamoyl]-phenyl-methyl}-carbamoyl)-phenyl-methyl]-carbamic acid tert-butyl ester (prepared in step 1). MS (m/e): 506.2 [M+H]⁺.

### EXAMPLE 250

### (S)-2-Amino-N-{(R)-[(3,4-dimethyl-phenyl)-(2-p-tolyl-ethyl)-carbamoyl]-phenyl-methyl}-propionamide

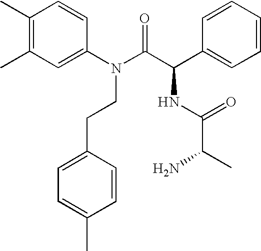

In analogy to the procedure described for the synthesis of example 53 (step 3), the title compound (−ve rotation) was prepared from [(S)-1-({[(3,4-Dimethyl-phenyl)-(2-p-tolyl-ethyl)-carbamoyl]-phenyl-methyl}-carbamoyl)-ethyl]-carbamic acid tert-butyl ester (prepared in example 172). MS (m/e): 444.2 [M+H]⁺.

### EXAMPLE 251

### (S)-2-Amino-N-{(S)-[(3,4-dimethyl-phenyl)-(2-p-tolyl-ethyl)-carbamoyl]-phenyl-methyl}-propionamide

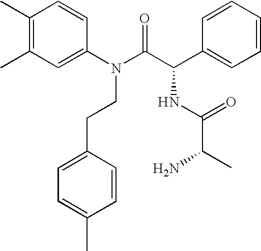

In analogy to the procedure described for the synthesis of example 53 (step 3), the title compound (+ve rotation) was prepared from [(S)-1-({[(3,4-Dimethyl-phenyl)-(2-p-tolyl-ethyl)-carbamoyl]-phenyl-methyl}-carbamoyl)-ethyl]-carbamic acid tert-butyl ester (prepared in example 172). MS (m/e): 444.2 [M+H]⁺.

1. A compound of formula I 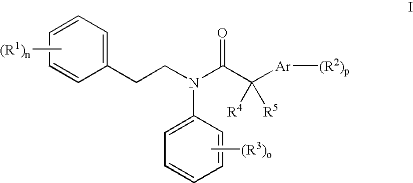
wherein
Ar is aryl or heteroaryl;
R¹ is hydrogen, halogen, lower alkyl, lower alkyl substituted by halogen, lower alkoxy, lower alkoxy substituted by halogen, cyano, SO₂-lower alkyl or hydroxy;
R² is hydrogen, halogen, lower alkyl, lower alkyl substituted by halogen, lower alkoxy, lower alkoxy substituted by halogen, cyano, S-lower alkyl, SO₂-lower alkyl, NO₂ or hydroxy;
R³ is hydrogen, halogen, lower alkyl, lower alkyl substituted by halogen, lower alkoxy, —(CH₂)ₘ—O-lower alkyl, lower alkoxy substituted by halogen, 3-hydroxy-oxetan-3-yl, cyano or SO₂-lower alkyl;
or if o is 2, R³ in the 3 and 4 positions together with the carbon atoms to which they are attached can form an additional ring with the groups —O—CH₂—O—, —O—CF₂—CF₂—O—, —N═CH—S—, —O—CF₂—O—, —(CH₂)₄—, —NH—C(O)—NH—, —O—(CH₂)₂— or —(CH₂)₂—O—;
R⁴ and R⁵ are each independently hydrogen, —(CR″₂)ₘOH, lower alkyl, lower alkoxy, —NRR′, or is —(CH₂)_{q}-heterocycloalkyl optionally substituted by hydroxy, or R⁴ and R⁵ are together ═O or ═N—OH;
R and R′ are each independently hydrogen, lower alkyl, C(O)H, —(CR″₂)ₘ—OH, —(CR″₂)ₘ—NR″₂, —(CR″₂)ₘ—NR″—C(O)-lower alkyl, —(CR″₂)ₘ—O-lower alkyl, —(CR″₂)ₘ—O-lower alkenyl, —C(O)O-lower alkyl, —C(O)—CR″₂—NH—C(O)O-lower alkyl, —C(O)—CR″₂—NR″₂, or —(CH₂)_{q}-heterocycloalkyl or —(CH₂)_{q}-furan-2-yl;
R″ are each independently hydrogen, lower alkoxy, phenyl or lower alkyl;
n is 1, 2, 3 or 4;
o is 1, 2 or 3;
p is 1, 2 or 3;
m is 1, 2 or 3; and
q is 0 or 1;
or a pharmaceutically suitable acid addition salt, optically pure enantiomer, racemate or diastereomeric mixture thereof,
with the exception of
N-(4-fluorophenyl)-3-hydroxy-N-[2-(3-methoxyphenyl)ethyl]benzeneacetamide,
N-(4-fluorophenyl)-4-hydroxy-N-[2-(3-methoxyphenyl)ethyl]-3-methyl benzeneacetamide,
4-bromo-N-[2-(3-methoxyphenyl)ethyl]-N-phenyl-benzeneacetamide, and
N-(4-methoxyphenyl)-N-[2-(3-methoxyphenyl)ethyl]benzeneacetamide.

2. The compound of claim 1, wherein Ar is phenyl.

3. The compound of claim 2, wherein one of R⁴ and R⁵ is hydroxy.

4. The compound of claim 3, selected from the group consisting of
N-(3,4-dimethoxyphenyl)-2-hydroxy-2-(2-methoxyphenyl)-N-{2-[4-(trifluoromethyl)phenyl]ethyl}acetamide,
(2S)—N-(3,4-dimethoxyphenyl)-2-hydroxy-2-phenyl-N-{2-[4-(trifluoromethyl)phenyl]ethyl}acetamide,
N-(3,4-dimethoxyphenyl)-2-(4-fluorophenyl)-2-hydroxy-N-{2-[4-(trifluoromethyl)phenyl]ethyl}acetamide,
N-(3,4-dimethoxyphenyl)-2-hydroxy-2-(3-hydroxy-4-methoxyphenyl)-N-{2-[4-(trifluoromethyl)phenyl]ethyl}acetamide,
2-(4-chlorophenyl)-N-(3,4-dimethoxyphenyl)-2-hydroxy-N-{2-[4-(trifluoromethyl)phenyl]ethyl}acetamide,
N-(3,4-dimethoxyphenyl)-2-(2-fluorophenyl)-2-hydroxy-N-{2-[4-(trifluoromethyl)phenyl]ethyl}acetamide,
(S)—N-(3,4-dimethyl-phenyl)-2-hydroxy-2-phenyl-N-[2-(4-trifluoromethyl-phenyl)-ethyl]-acetamide, and
(S)—N-(3,4-dimethyl-phenyl)-2-(4-fluoro-phenyl)-2-hydroxy-N-[2-(4-trifluoromethyl-phenyl)-ethyl]-acetamide.

5. The compound of claim 2, wherein both of R⁴ and R⁵ are hydrogen.

6. The compound of claim 5, wherein the compound is
N-(3,4-dimethoxyphenyl)-2-(2-methoxyphenyl)-N-{2-[4-(trifluoromethyl)phenyl]ethyl}acetamide.

7. The compound of claim 2, wherein one of R⁴ and R⁵ is NH₂.

8. The compound of claim 7, selected from the group consisting of
2-amino-N-(3,4-dimethoxy-phenyl)-2-(2-methoxy-phenyl)-N-[2-(4-trifluoromethyl-phenyl)-ethyl]-acetamide,
2-amino-2-(2-methoxy-phenyl)-N-(3-methoxy-phenyl)-N-[2-(4-trifluoromethyl-phenyl)-ethyl]-acetamide,
(2-amino-N-benzo[1,3]dioxol-5-yl-2-(2-methoxy-phenyl)-N-[2-(4-trifluoromethyl-phenyl)-ethyl]-acetamide,
(2-amino-N-(3-fluoro-4-methoxy-phenyl)-2-(2-methoxy-phenyl)-N-[2-(4-trifluoromethyl-phenyl)-ethyl]-acetamide,
2-amino-N-(3-chloro-4-methoxy-phenyl)-2-(2-methoxy-phenyl)-N-[2-(4-trifluoromethyl-phenyl)-ethyl]-acetamide,
2-amino-2-(2-methoxy-phenyl)-N-(2,2,3,3-tetrafluoro-2,3-dihydro-benzo[1,4]dioxin-6-yl)-N-[2-(4-trifluoromethyl-phenyl)-ethyl]-acetamide,
2-amino-N-(3,4-dimethoxy-phenyl)-2-phenyl-N-[2-(4-trifluoromethyl-phenyl)-ethyl]-acetamide,
2-amino-N-benzo[1,3]dioxol-5-yl-2-phenyl-N-[2-(4-trifluoromethyl-phenyl)-ethyl]-acetamide,
2-amino-N-(5-methoxy-2-methyl-phenyl)-2-phenyl-N-[2-(4-trifluoromethyl-phenyl)-ethyl]-acetamide, and
2-amino-N-(3-methoxy-phenyl)-2-phenyl-N-[2-(4-trifluoromethyl-phenyl)-ethyl]-acetamide.

9. The compound of claim 7, selected from the group consisting of
2-amino-N-(3,4-dimethoxy-phenyl)-2-(4-fluoro-phenyl)-N-[2-(4-trifluoromethyl-phenyl)-ethyl]-acetamide,
2-amino-N-(3,4-dimethoxy-phenyl)-2-(4-fluoro-phenyl)-N-[2-(4-trifluoromethyl-phenyl)-ethyl]-acetamide,
2-amino-2-(3-chloro-phenyl)-N-(3,4-dimethoxy-phenyl)-N-[2-(4-trifluoromethyl-phenyl)-ethyl]-acetamide,
2-amino-N-(3-fluoro-4-methoxy-phenyl)-2-phenyl-N-[2-(4-trifluoromethyl-phenyl)-ethyl]-acetamide,
2-amino-N-(4-chloro-3-methoxy-phenyl)-2-phenyl-N-[2-(4-trifluoromethyl-phenyl)-ethyl]-acetamide,
2-amino-N-(3-chloro-4-methoxy-phenyl)-2-phenyl-N-[2-(4-trifluoromethyl-phenyl)-ethyl]-acetamide,
2-amino-N-(3,4-diethoxy-phenyl)-2-phenyl-N-[2-(4-trifluoromethyl-phenyl)-ethyl]-acetamide,
2-amino-2-phenyl-N-p-tolyl-N-[2-(4-trifluoromethyl-phenyl)-ethyl]-acetamide,
2-amino-2-phenyl-N-m-tolyl-N-[2-(4-trifluoromethyl-phenyl)-ethyl]-acetamide, and
2-amino-N-(4-methoxy-phenyl)-2-phenyl-N-[2-(4-trifluoromethyl-phenyl)-ethyl]-acetamide.

10. The compound of claim 7, selected from the group consisting of
2-amino-N-[2-(3,4-dimethoxy-phenyl)-ethyl]-2-phenyl-N-(4-trifluoromethoxy-phenyl)-acetamide,
2-amino-N-(4-fluoro-3-methyl-phenyl)-2-phenyl-N-[2-(4-trifluoromethyl-phenyl)-ethyl]-acetamide,
2-amino-N-(3,4-dimethyl-phenyl)-2-phenyl-N-[2-(4-trifluoromethyl-phenyl)-ethyl]-acetamide,
2-amino-N-(3,4-dimethoxy-phenyl)-2-(3-fluoro-phenyl)-N-[2-(4-trifluoromethyl-phenyl)-ethyl]-acetamide,
2-amino-N-(4-fluoro-3-methyl-phenyl)-2-phenyl-N-(2-p-tolyl-ethyl)-acetamide,
2-amino-N-(3,4-dimethyl-phenyl)-2-phenyl-N-(2-p-tolyl-ethyl)-acetamide,
2-amino-N-[2-(4-chloro-phenyl)-ethyl]-N-(3,4-dimethyl-phenyl)-2-phenyl-acetamide,
2-amino-N-[3-(3-hydroxy-oxetan-3-yl)-phenyl]-2-phenyl-N-[2-(4-trifluoromethyl-phenyl)-ethyl]-acetamide,
(S)-2-amino-N-(3,4-dimethoxy-phenyl)-2-(2-methoxy-phenyl)-N-[2-(4-trifluoromethyl-phenyl)-ethyl]-acetamide, and
2-amino-N-(4-fluoro-3-methoxy-phenyl)-2-phenyl-N-[2-(4-trifluoromethyl-phenyl)-ethyl]-acetamide.

11. The compound of claim 7, selected from the group consisting of
2-amino-N-(4-difluoromethoxy-phenyl)-2-phenyl-N-[2-(4-trifluoromethyl-phenyl)-ethyl]-acetamide,
2-amino-N-(2-chloro-5-methoxy-phenyl)-2-phenyl-N-[2-(4-trifluoromethyl-phenyl)-ethyl]-acetamide,
2-amino-2-phenyl-N-(5,6,7,8-tetrahydro-naphthalen-2-yl)-N-[2-(4-trifluoromethyl-phenyl)-ethyl]-acetamide,
(S)-2-amino-N-(3,4-dimethyl-phenyl)-2-phenyl-N-[2-(4-trifluoromethyl-phenyl)-ethyl]-acetamide,
(S)-2-amino-N-(3,4-diethoxy-phenyl)-2-phenyl-N-[2-(4-trifluoromethyl-phenyl)-ethyl]-acetamide,
(S)-2-amino-N-(3,4-dimethyl-phenyl)-2-(4-fluoro-phenyl)-N-(2-p-tolyl-ethyl)-acetamide,
(S)-2-amino-2-phenyl-N-p-tolyl-N-[2-(4-trifluoromethyl-phenyl)-ethyl]-acetamide,
(S)-2-amino-N-(4-difluoromethoxy-phenyl)-2-phenyl-N-[2-(4-trifluoromethyl-phenyl)-ethyl]-acetamide,
(S)-2-amino-2-phenyl-N-(5,6,7,8-tetrahydro-naphthalen-2-yl)-N-[2-(4-trifluoromethyl-phenyl)-ethyl]-acetamide, and
(S)-2-amino-N-(3-methoxy-phenyl)-2-phenyl-N-[2-(4-trifluoromethyl-phenyl)-ethyl]-acetamide.

12. The compound of claim 7, selected from the group consisting of
(S)-2-amino-N-(3,4-dimethyl-phenyl)-2-(4-fluoro-phenyl)-N-[2-(4-trifluoromethyl-phenyl)-ethyl]-acetamide
(S)-2-amino-N-(3-methoxy-4-methyl-phenyl)-2-phenyl-N-[2-(4-trifluoromethyl-phenyl)-ethyl]-acetamide,
(S)-2-amino-N-(3-chloro-4-difluoromethoxy-phenyl)-2-(4-fluoro-phenyl)-N-[2-(4-trifluoromethyl-phenyl)-ethyl]-acetamide,
(S)-2-amino-N-(3-chloro-4-ethoxy-phenyl)-2-(4-fluoro-phenyl)-N-[2-(4-trifluoromethyl-phenyl)-ethyl]-acetamide,
(S)-2-amino-N-(3,4-dimethoxy-phenyl)-2-phenyl-N-[2-(4-trifluoromethyl-phenyl)-ethyl]-acetamide hydrochloride,
(R)-2-amino-N-(3,4-dimethoxy-phenyl)-2-phenyl-N-[2-(4-trifluoromethyl-phenyl)-ethyl]-acetamide,
(S)-2-amino-N-(3,4-dimethyl-phenyl)-N-[2-(4-fluoro-phenyl)-ethyl]-2-phenyl-acetamide,
(S)-2-amino-N-(3,4-dimethyl-phenyl)-2-phenyl-N-[2-(4-trifluoromethoxy-phenyl)-ethyl]-acetamide,
(S)-2-amino-N-(3,4-dimethyl-phenyl)-N-[2-(2-fluoro-phenyl)-ethyl]-2-phenyl-acetamide, and
(S)-2-amino-N-(3,4-dimethyl-phenyl)-2-phenyl-N-(2-m-tolyl-ethyl)-acetamide.

13. The compound of claim 7, selected from the group consisting of
(S)-2-amino-N-(3,4-dimethyl-phenyl)-N-[2-(3-fluoro-phenyl)-ethyl]-2-phenyl-acetamide,
(S)-2-amino-N-[2-(3-chloro-phenyl)-ethyl]-N-(3,4-dimethyl-phenyl)-2-phenyl-acetamide,
(S)-2-amino-N-(3,4-dimethyl-phenyl)-2-phenyl-N-[2-(3-trifluoromethyl-phenyl)-ethyl]-acetamide,
(S)-2-amino-N-(3,4-dimethyl-phenyl)-2-phenyl-N-(2-o-tolyl-ethyl)-acetamide,
(S)-2-amino-N-(3,4-dimethyl-phenyl)-N-[2-(3-fluoro-4-trifluoromethyl-phenyl)-ethyl]-2-phenyl-acetamide,
(S)-2-amino-N-(3,4-dimethyl-phenyl)-N-[2-(4-fluoro-3-trifluoromethyl-phenyl)-ethyl]-2-phenyl-acetamide,
(S)-2-amino-N-[2-(2,4-difluoro-phenyl)-ethyl]-N-(3,4-dimethyl-phenyl)-2-phenyl-acetamide,
(S)-2-amino-N-[2-(3,4-difluoro-phenyl)-ethyl]-N-(3,4-dimethyl-phenyl)-2-phenyl-acetamide,
(S)-2-amino-N-(3,4-dimethyl-phenyl)-N-[2-(3-fluoro-4-methyl-phenyl)-ethyl]-2-phenyl-acetamide, and
(S)-2-amino-N-[2-(2,3-difluoro-phenyl)-ethyl]-N-(3,4-dimethyl-phenyl)-2-phenyl-acetamide.

14. The compound of claim 7, selected from the group consisting of
(S)-2-amino-N-[2-(4-chloro-2-fluoro-phenyl)-ethyl]-N-(3,4-dimethyl-phenyl)-2-phenyl-acetamide,
(S)-2-amino-N-(3,4-dimethyl-phenyl)-N-[2-(2-fluoro-5-trifluoromethyl-phenyl)-ethyl]-2-phenyl-acetamide,
(S)-2-amino-N-(3,4-dimethyl-phenyl)-N-[2-(2-methoxy-phenyl)-ethyl]-2-phenyl-acetamide,
(S)-2-amino-N-(3,4-dimethyl-phenyl)-N-[2-(2-fluoro-3-trifluoromethyl-phenyl)-ethyl]-2-phenyl-acetamide,
(S)-2-amino-N-(3,4-dimethyl-phenyl)-N-[2-(2-fluoro-4-trifluoromethyl-phenyl)-ethyl]-2-phenyl-acetamide,
(S)-2-amino-N-[2-(2,3-difluoro-4-trifluoromethyl-phenyl)-ethyl]-N-(3,4-dimethyl-phenyl)-2-phenyl-acetamide,
(S)-2-amino-N-(3,4-dimethyl-phenyl)-2-phenyl-N-[2-(2-trifluoromethoxy-phenyl)-ethyl]-acetamide,
(S)-2-amino-N-(3,4-dimethyl-phenyl)-N-phenethyl-2-phenyl-acetamide,
(S)-2-amino-N-[2-(4-chloro-3-fluoro-phenyl)-ethyl]-N-(3,4-dimethyl-phenyl)-2-phenyl-acetamide, and
(S)-2-amino-N-(3,4-dimethyl-phenyl)-N-[2-(3-fluoro-5-trifluoromethyl-phenyl)-ethyl]-2-phenyl-acetamide.

15. The compound of claim 7, selected from the group consisting of
(S)-2-amino-N-(3,4-dimethyl-phenyl)-2-phenyl-N-[2-(2,3,6-trifluoro-4-trifluoromethyl-phenyl)-ethyl]-acetamide,
(S)-2-amino-N-[2-(2,5-dichloro-phenyl)-ethyl]-N-(3,4-dimethyl-phenyl)-2-phenyl-acetamide,
(S)-2-amino-N-[2-(3-chloro-2-fluoro-phenyl)-ethyl]-N-(3,4-dimethyl-phenyl)-2-phenyl-acetamide,
(S)-2-amino-N-[2-(2,4-dichloro-phenyl)-ethyl]-N-(3,4-dimethyl-phenyl)-2-phenyl-acetamide,
(S)-2-amino-N-(3,4-dimethyl-phenyl)-N-[2-(3-hydroxy-phenyl)-ethyl]-2-phenyl-acetamide,
(S)-2-amino-N-[2-(3,5-difluoro-phenyl)-ethyl]-N-(3,4-dimethyl-phenyl)-2-phenyl-acetamide,
(S)-2-amino-N-[2-(3-chloro-5-fluoro-phenyl)-ethyl]-N-(3,4-dimethyl-phenyl)-2-phenyl-acetamide,
(S)-2-amino-N-[2-(4-difluoromethoxy-phenyl)-ethyl]-N-(3,4-dimethyl-phenyl)-2-phenyl-acetamide,
(S)-2-amino-N-[2-(4-cyano-phenyl)-ethyl]-N-(3,4-dimethyl-phenyl)-2-phenyl-acetamide,
(S)-2-amino-N-(2,3-dihydro-benzofuran-5-yl)-N-[2-(4-fluoro-phenyl)-ethyl]-2-phenyl-acetamide,
(S)-2-amino-N-(2,3-dihydro-benzofuran-6-yl)-N-[2-(4-fluoro-phenyl)-ethyl]-2-phenyl-acetamide, and
(S)-2-amino-N-(4-ethyl-phenyl)-N-[2-(4-fluoro-phenyl)-ethyl]-2-phenyl-acetamide.

16. The compound of claim 2, wherein one of R⁴ and R⁵ is NRR′ and R and R′ are other than hydrogen.

17. The compound of claim 16, selected from the group consisting of
N-(3,4-dimethoxy-phenyl)-2-(2-methoxy-phenyl)-2-methylamino-N-[2-(4-trifluoromethyl-phenyl)-ethyl]-acetamide,
N-(3,4-dimethoxy-phenyl)-2-methylamino-2-phenyl-N-[2-(4-trifluoromethyl-phenyl)-ethyl]-acetamide,
(S)—N-(3,4-dimethoxy-phenyl)-2-(oxetan-3-ylamino)-2-phenyl-N-[2-(4-trifluoromethyl-phenyl)-ethyl]-acetamide,
(S)—N-(3,4-dimethoxy-phenyl)-2-(2-methoxy-phenyl)-2-methylamino-N-[2-(4-trifluoromethyl-phenyl)-ethyl]-acetamide,
N-(3,4-dimethyl-phenyl)-2-(2-hydroxy-ethylamino)-2-phenyl-N-(2-p-tolyl-ethyl)-acetamide,
N-(3,4-dimethyl-phenyl)-2-(2-methoxy-1-methyl-ethylamino)-2-phenyl-N-(2-p-tolyl-ethyl)-acetamide,
N-(3,4-dimethyl-phenyl)-2-(2-hydroxy-propylamino)-2-phenyl-N-(2-p-tolyl-ethyl)-acetamide,
N-(3,4-dimethyl-phenyl)-2-(2-hydroxy-1-methyl-ethylamino)-2-phenyl-N-(2-p-tolyl-ethyl)-acetamide,
N-(3,4-dimethyl-phenyl)-2-(1-hydroxymethyl-2-methyl-propylamino)-2-phenyl-N-(2-p-tolyl-ethyl)-acetamide,
N-(3,4-dimethyl-phenyl)-2-(1-methoxymethyl-propylamino)-2-phenyl-N-(2-p-tolyl-ethyl)-acetamide,
2-(2-acetylamino-ethylamino)-N-(3,4-dimethyl-phenyl)-2-phenyl-N-(2-p-tolyl-ethyl)-acetamide,
N-(3,4-dimethyl-phenyl)-2-phenyl-2-[(tetrahydro-furan-2-ylmethyl)-amino]-N-(2-p-tolyl-ethyl)-acetamide, and
2-(2,2-dimethoxy-ethylamino)-N-(3,4-dimethyl-phenyl)-2-phenyl-N-(2-p-tolyl-ethyl)-acetamide.

18. The compound of claim 16, selected from the group consisting of
N-(3,4-dimethyl-phenyl)-2-[(furan-2-ylmethyl)-amino]-2-phenyl-N-(2-p-tolyl-ethyl)-acetamide,
N-(3,4-dimethyl-phenyl)-2-(2-hydroxy-butylamino)-2-phenyl-N-(2-p-tolyl-ethyl)-acetamide,
N-(3,4-dimethyl-phenyl)-2-(2-hydroxy-1,1-dimethyl-ethylamino)-2-phenyl-N-(2-p-tolyl-ethyl)-acetamide,
N-(3,4-dimethyl-phenyl)-2-[([1,3]dioxolan-2-ylmethyl)-amino]-2-phenyl-N-(2-p-tolyl-ethyl)-acetamide,
N-(3,4-dimethyl-phenyl)-2-phenyl-2-{[(S)-1-(tetrahydro-furan-2-yl)methyl]-amino}-N-(2-p-tolyl-ethyl)-acetamide,
N-(3,4-dimethyl-phenyl)-2-phenyl-N-(2-p-tolyl-ethyl)-2-(2-vinyloxy-ethylamino)-acetamide,
N-(3,4-dimethyl-phenyl)-2-(2-ethoxy-ethylamino)-2-phenyl-N-(2-p-tolyl-ethyl)-acetamide,
(S)—N-(3,4-dimethyl-phenyl)-2-(2-methoxy-phenyl)-2-methylamino-N-[2-(4-trifluoromethyl-phenyl)-ethyl]-acetamide,
(R,S)—N-(3,4-dimethyl-phenyl)-2-ethoxy-2-phenyl-N-[2-(4-trifluoromethyl-phenyl)-ethyl]-acetamide,
(S)-2-(2-amino-acetylamino)-N-(3,4-dimethyl-phenyl)-2-phenyl-N-(2-p-tolyl-ethyl)-acetamide,
(S)-2-((S)-2-amino-2-phenyl-acetylamino)-N-(3,4-dimethyl-phenyl)-2-phenyl-N-(2-p-tolyl-ethyl)-acetamide, and
(S)-2-amino-N-{(S)-[(3,4-dimethyl-phenyl)-(2-p-tolyl-ethyl)-carbamoyl]-phenyl-methyl}-propionamide.

19. The compound of claim 2, wherein R⁴ and R⁵ are together ═O or ═N—OH.

20. The compound of claim 19, selected from the group consisting of
N-(3,4-dimethyl-phenyl)-2-[hydroxyimino]-2-phenyl-N-[2-(2,3,6-trifluoro-4-trifluoromethyl-phenyl)-ethyl]-acetamide and
N-(3,4-dimethyl-phenyl)-2-oxo-2-phenyl-N-[2-(4-trifluoromethyl-phenyl)-ethyl]-acetamide.

21. The compound of claim 1, wherein Ar is heteroaryl.

22. The compound of claim 21, selected from the group consisting of
2-amino-2-(5-chloro-thiophen-2-yl)-N-[2-(3,4-difluoro-phenyl)-ethyl]-N-(3,4-dimethyl-phenyl)-acetamide hydrochloride,
2-amino-N-[2-(3,4-difluoro-phenyl)-ethyl]-N-(3,4-dimethyl-phenyl)-2-thiophen-3-yl-acetamide hydrochloride, and
N-(3,4-dimethyl-phenyl)-2-(2-methyl-benzoimidazol-1-yl)-N-[2-(4-trifluoromethyl-phenyl)-ethyl]-acetamide.

23. A pharmaceutical composition comprising a therapeutically effective amount of a compound of formula I 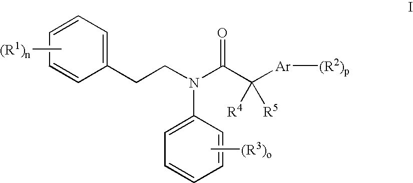
wherein
Ar is aryl or heteroaryl;
R¹ is hydrogen, halogen, lower alkyl, lower alkyl substituted by halogen, lower alkoxy, lower alkoxy substituted by halogen, cyano, SO₂-lower alkyl or hydroxy;
R² is hydrogen, halogen, lower alkyl, lower alkyl substituted by halogen, lower alkoxy, lower alkoxy substituted by halogen, cyano, S-lower alkyl, SO₂-lower alkyl, NO₂ or hydroxy;
R³ is hydrogen, halogen, lower alkyl, lower alkyl substituted by halogen, lower alkoxy, —(CH₂)ₘ—O-lower alkyl, lower alkoxy substituted by halogen, 3-hydroxy-oxetan-3-yl, cyano or SO₂-lower alkyl;
or if o is 2, R³ in the 3 and 4 positions together with the carbon atoms to which they are attached can form an additional ring with the groups —O—CH₂—O—, —O—CF₂—CF₂—O—, —N═CH—S—, —O—CF₂—O—, —(CH₂)₄—, —NH—C(O)—NH—, —O—(CH₂)₂— or —(CH₂)₂—O—;
R⁴ and R⁵ are each independently hydrogen, —(CR″₂)ₘOH, lower alkyl, lower alkoxy, —NRR′, or is —(CH₂)_{q}-heterocycloalkyl optionally substituted by hydroxy, or R⁴ and R⁵ are together ═O or ═N—OH;
R and R′ are each independently hydrogen, lower alkyl, C(O)H, —(CR″₂)ₘ—OH, —(CR″₂)ₘ—NR″₂, —(CR″₂)ₘ—NR″—C(O)-lower alkyl, —(CR″₂)ₘ—O-lower alkyl, —(CR″₂)ₘ—O-lower alkenyl, —C(O)O-lower alkyl, —C(O)—CR″₂—NH—C(O)O-lower alkyl, —C(O)—CR″₂—NR″₂, or —(CH₂)_{q}-heterocycloalkyl or —(CH₂)_{q}-furan-2-yl;
R″ are each independently hydrogen, lower alkoxy, phenyl or lower alkyl;
n is 1, 2, 3 or 4;
o is 1, 2 or 3;
p is 1, 2 or 3;
m is 1, 2 or 3; and
q is 0 or 1;
or a pharmaceutically suitable acid addition salt, optically pure enantiomer, racemate or diastereomeric mixture thereof,
with the exception of
N-(4-fluorophenyl)-3-hydroxy-N-[2-(3-methoxyphenyl)ethyl]benzeneacetamide,
N-(4-fluorophenyl)-4-hydroxy-N-[2-(3-methoxyphenyl)ethyl]-3-methyl benzeneacetamide,
4-bromo-N-[2-(3-methoxyphenyl)ethyl]-N-phenyl-benzeneacetamide, and
N-(4-methoxyphenyl)-N-[2-(3-methoxyphenyl)ethyl]benzeneacetamide
and a pharmaceutically acceptable carrier.
